SEQUENCE LISTING (117 sequences)

SEQ ID NO: 1 - protein, 121 aa, Artificial Sequence
  Heavy chain variable region
QVQLVQSGSELKKPGASVKVSCKASGYTFTNFAMNWVRRAPGQGLEWMGWINTNTGNPTYAQGFTGRFVFSLDTSVNTAYLQISSLKAEDTAVYYCARDWGVIGGHYMDVWGKGTTVTVSS

SEQ ID NO: 2 - protein, 5 aa, Artificial Sequence
  HCDR1
NFAMN

SEQ ID NO: 3 - protein, 17 aa, Artificial Sequence
  HCDR2
WINTNTGNPTYAQGFTG

SEQ ID NO: 4 - protein, 12 aa, Artificial Sequence
  HCDR3
DWGVIGGHYMDV

SEQ ID NO: 5 - protein, 121 aa, Artificial Sequence
  Heavy chain variable region
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDSDGYGPKAFDYWGQGTLVTVSS

SEQ ID NO: 6 - protein, 5 aa, Artificial Sequence
  HCDR1
SYGIS

SEQ ID NO: 7 - protein, 17 aa, Artificial Sequence
  HCDR2
WISAYNGNTNYAQKLQG

SEQ ID NO: 8 - protein, 12 aa, Artificial Sequence
  HCDR3
DSDGYGPKAFDY

SEQ ID NO: 9 - protein, 123 aa, Artificial Sequence
  Heavy chain variable region
EVQLVQSGAEVKKPGESLKISCKGSGYSFTSYWIGWVRQMPGKGLEWMGIIYPDDSDTRYSPSFQGQVTISADKSSSTAYLQWSSLKASDTAMYYCASFYTGIVGATGAFDVWGQGTTVTVSS

SEQ ID NO: 10 - protein, 5 aa, Artificial Sequence
  HCDR1
SYWIG

SEQ ID NO: 11 - protein, 17 aa, Artificial Sequence
  HCDR2
IIYPDDSDTRYSPSFQG

SEQ ID NO: 12 - protein, 14 aa, Artificial Sequence
  HCDR3
FYTGIVGATGAFDV

SEQ ID NO: 13 - protein, 128 aa, Artificial Sequence
  Heavy chain variable region
QVQLVQSGAEVKKPGSSVKVSCKASGGTFSSDAISWVRQAPGQGLEWMGGMIPILGTANYAQKFQGRVTITADRSTSTAYMELSSLRSEDTAVYYCVRGATYYYGSGTYYSINWFDPWGQGTLVTVSS

SEQ ID NO: 14 - protein, 5 aa, Artificial Sequence
  HCDR1
SDAIS

SEQ ID NO: 15 - protein, 17 aa, Artificial Sequence
  HCDR2
GMIPILGTANYAQKFQG

SEQ ID NO: 16 - protein, 15 aa, Artificial Sequence
  HCDR3
GATYYYGSGTYYSIN

SEQ ID NO: 17 - protein, 122 aa, Artificial Sequence
  Heavy chain variable region
QVQLVQSGSELKKPGASVKVSCRASGYTFTNFAMTWVRQAPGQGPEYMGWINTNTGNPTYAQGFTGRFVFSLDTSVNTAYLQISSLKAEDTAVYYCARDWASVMVRGDLDYWGQGTLVTVSS

SEQ ID NO: 18 - protein, 4 aa, Artificial Sequence
  HCDR1
NFAM

SEQ ID NO: 19 - protein, 13 aa, Artificial Sequence
  HCDR3
DWASVMVRGDLDY

SEQ ID NO: 20 - protein, 126 aa, Artificial Sequence
  Heavy chain variable region
QVQLVQSGAEVKKPGASVKVSCKVSGYTLSELSIHWVRQAPGKGVEWMGGFYPEDVEPIYARKFQGRVTMTEDTSTDTAYMELNSLRSEDTAVYYCAAEGFDNYGSGIRGNWFDPWGQGTLVTVSS

SEQ ID NO: 21 - protein, 5 aa, Artificial Sequence
  HCDR1
ELSIH

SEQ ID NO: 22 - protein, 17 aa, Artificial Sequence
  HCDR2
GFYPEDVEPIYARKFQG

SEQ ID NO: 23 - protein, 17 aa, Artificial Sequence
  HCDR3
EGFDNYGSGIRGNWFDP

SEQ ID NO: 24 - protein, 122 aa, Artificial Sequence
  Heavy chain variable region
EVQLVQSGAEVKKPGASVKVSCKVSGYTLTELSMHWVRQSPGKGLEWMGSFYPEDGETIYAQKFQGRITMTEDTSADTAYMELSSLRSEDTAVYYCATEGVGVIRGNWFDPWGQGTLVTVSS

SEQ ID NO: 25 - protein, 5 aa, Artificial Sequence
  HCDR1
ELSMH

SEQ ID NO: 26 - protein, 17 aa, Artificial Sequence
  HCDR2
SFYPEDGETIYAQKFQG

SEQ ID NO: 27 - protein, 13 aa, Artificial Sequence
  HCDR3
EGVGVIRGNWFDP

SEQ ID NO: 28 - protein, 123 aa, Artificial Sequence
  Heavy chain variable region
EVQLVQSGAEVKKPGESLKISCKGSGYSFTSYWIGWVRQMPGKGLEWMGIIFPDDSDTRYSPSFQGQVTISADKSISTAYLQWSSLKPSDTAMYYCVRLGGYSGYAEDFVDFWGQGTLVTVSS

SEQ ID NO: 29 - protein, 17 aa, Artificial Sequence
  HCDR2
IIFPDDSDTRYSPSFQG

SEQ ID NO: 30 - protein, 15 aa, Artificial Sequence
  HCDR3
RLGGYSGYAEDFVDF

SEQ ID NO: 31 - protein, 124 aa, Artificial Sequence
  Heavy chain variable region
EVQLVQSGAEVKKPGASVKVSCKVSGYTLTKLSMHWVRQAPGKGLEWMGGFEPEDGETINAQKFQGRVTMTEDTSTDTAYMELSSLRSEDTAVYYCATDLRLGASYYYSYMDVWGRGTMVTVSS

SEQ ID NO: 32 - protein, 5 aa, Artificial Sequence
  HCDR1
KLSMH

SEQ ID NO: 33 - protein, 17 aa, Artificial Sequence
  HCDR2
GFEPEDGETINAQKFQG

SEQ ID NO: 34 - protein, 15 aa, Artificial Sequence
  HCDR3
DLRLGASYYYSYMDV

SEQ ID NO: 35 - protein, 121 aa, Artificial Sequence
  Heavy chain variable region
QITLKESGPTLVKPTQTLTLSCTFSGFSLSTSGMSVGWIRQPPGKALEWLALIYWNDDKYFSPSLKSRLTITKDTSKNQVVLTLTNMDPVDTATYYCAHTLWGSDDVFDVWGQGTMVTVSS

SEQ ID NO: 36 - protein, 7 aa, Artificial Sequence
  HCDR1
TSGMSVG

SEQ ID NO: 37 - protein, 16 aa, Artificial Sequence
  HCDR2
LIYWNDDKYFSPSLKS

SEQ ID NO: 38 - protein, 11 aa, Artificial Sequence
  HCDR3
TLWGSDDVFDV

SEQ ID NO: 39 - protein, 123 aa, Artificial Sequence
  Heavy chain variable region
EVQLVQSGAEVKKPGESLKISCKVSGYSFTNYWIGWVRQMPGKGLEWMGIIYPGDSDTRYSPSFQGQVTISADKSISTAYLQWHTLKASDTAMYYCARHQGYSFSGSHIDDYWGQGTLVTVSS

SEQ ID NO: 40 - protein, 5 aa, Artificial Sequence
  HCDR1
NYWIG

SEQ ID NO: 41 - protein, 17 aa, Artificial Sequence
  HCDR2
IIYPGDSDTRYSPSFQG

SEQ ID NO: 42 - protein, 14 aa, Artificial Sequence
  HCDR3
HQGYSFSGSHIDDY

SEQ ID NO: 43 - protein, 123 aa, Artificial Sequence
  Heavy chain variable region
EVQLVQSGAEVRKPGESLKISCKGSGYSFTTYWIGWVRQMPGKGLEWMGIIYPGDSDTRYSPSFQGQVTISADKSISTVYLQWSSLKASDTAMYYCARHAGFIITSQNIDDYWGQGTLVTVSS

SEQ ID NO: 44 - protein, 5 aa, Artificial Sequence
  HCDR1
TYWIG

SEQ ID NO: 45 - protein, 14 aa, Artificial Sequence
  HCDR3
HAGFIITSQNIDDY

SEQ ID NO: 46 - protein, 121 aa, Artificial Sequence
  Heavy chain variable region
EVQLVQSGSELKKPGASVKVSCKASGYTFTNFAMNWVRQAPGQGLEWMGWINTNTGNPTYAQDFTGRFVFSLDTSGNTAYLQISSLKAEDTAVYYCARDWGLVAIGYFDYWGQGTLVTVSS

SEQ ID NO: 47 - protein, 17 aa, Artificial Sequence
  HCDR2
WINTNTGNPTYAQDFTG

SEQ ID NO: 48 - protein, 12 aa, Artificial Sequence
  HCDR3
DWGLVAIGYFDY

SEQ ID NO: 49 - protein, 124 aa, Artificial Sequence
  Heavy chain variable region
QITLKESGPTLVKPTQTLTLTCTFSGFSLSTTGVGVNWIRQPPGEALEWLALIYWNDDTYYSPSLKSRLTITKDTSKNQVVLTMTNMDPVDTATYYCAHEGIIGFLGGNWFDPWGQGTLVTVSS

SEQ ID NO: 50 - protein, 7 aa, Artificial Sequence
  HCDR1
TTGVGVN

SEQ ID NO: 51 - protein, 16 aa, Artificial Sequence
  HCDR2
LIYWNDDTYYSPSLKS

SEQ ID NO: 52 - protein, 14 aa, Artificial Sequence
  HCDR3
EGIIGFLGGNWFDP

SEQ ID NO: 53 - protein, 125 aa, Artificial Sequence
  Heavy chain variable region
QVQLVQSGSELKKPGASVKVSCKASGYTFTSHAMNWVRQAPGQGLEWMGWINPNTGNPTYAQGFTGRFVFSLDTSVSTAYLQISSLKAEDTAVYYCARDRKYVTNWVFAEDFQHWGQGTLVTVSS

SEQ ID NO: 54 - protein, 5 aa, Artificial Sequence
  HCDR1
SHAMN

SEQ ID NO: 55 - protein, 17 aa, Artificial Sequence
  HCDR2
WINPNTGNPTYAQGFTG

SEQ ID NO: 56 - protein, 16 aa, Artificial Sequence
  HCDR3
DRKYVTNWVFAEDFQH

SEQ ID NO: 57 - protein, 125 aa, Artificial Sequence
  Heavy chain variable region
QVQLVQSGSELKKPGASVKVSCKASGYTFTSHAMNWVRQAPGQGLEWMGWINPNTGNPTYAQGFTGRFVFSLDTSVSTAYLQISSLKAEDTAVYYCAIDRGYMSNWVFAEYFPHWGQGTLVTVSS

SEQ ID NO: 58 - protein, 16 aa, Artificial Sequence
  HCDR3
DRGYMSNWVFAEYFPH

SEQ ID NO: 59 - protein, 125 aa, Artificial Sequence
  Heavy chain variable region
QVQLVQSGSELKKPGASVKVSCKASGYTFTSYAMNWVRQAPGQGLEWMGWINTNTGNPTYAQGFTGRFVFSLDTSVSTAYLQISSLKAEDTAVYYCATDRGYISSWVFAEDFQHWGQGTLVTVSS

SEQ ID NO: 60 - protein, 5 aa, Artificial Sequence
  HCDR1
SYAMN

SEQ ID NO: 61 - protein, 16 aa, Artificial Sequence
  HCDR3
DRGYISSWVFAEDFQH

SEQ ID NO: 62 - protein, 125 aa, Artificial Sequence
  Heavy chain variable region
QVQLVQSGSELKKPGASVKVSCTASGYTFTSYAMNWVRQAPGQRLEWMACVNPNTGSPTYAQGSTGRFVVSLDTSVSTAYLQISSLKAEDTAVYYCARDRKYVTNWVFAEDFQHWGHGTLVTVSS

SEQ ID NO: 63 - protein, 17 aa, Artificial Sequence
  HCDR2
CVNPNTGSPTYAQGSTG

SEQ ID NO: 64 - protein, 125 aa, Artificial Sequence
  Heavy chain variable region
QVQLVQSGSELKKPGASVKVSCKASGYTFTNYAMNWVRQAPGQGLEWMGWMNPNTGNPTYAQGSTGRFVVSLDTSVSTAYLQISSLKAEDTAVYYCARDRKYVTNWVFAEDFQHWGRGTLVTVSS

SEQ ID NO: 65 - protein, 5 aa, Artificial Sequence
  HCDR1
NYAMN

SEQ ID NO: 66 - protein, 17 aa, Artificial Sequence
  HCDR2
WMNPNTGNPTYAQGSTG

SEQ ID NO: 67 - protein, 125 aa, Artificial Sequence
  Heavy chain variable region
QVQLVQSGSELKKPGASVKVSCKASGYTFTNYAINWVRQAPGQGLEWMGWINPNTGNPTYAQGFTGRFVFSLDTSVSTAYLQISSLKAEDTAVYYCARDRKYVTNWVFAEDFQHWGRGTLVTVSS

SEQ ID NO: 68 - protein, 5 aa, Artificial Sequence
  HCDR1
NYAIN

SEQ ID NO: 69 - protein, 124 aa, Artificial Sequence
  Heavy chain variable region
EVQLVQSGAEVKKPGSSVKVSCKASGDTFNTYSITWVRQAPGQGLEWMGSIVPIFGTINNAQKFQGRVTITADKSANTAYMELSSLRSEDTAVYYCARDNTMVRGVDYYYMDVWGKGTMVTVSS

SEQ ID NO: 70 - protein, 5 aa, Artificial Sequence
  HCDR1
TYSIT

SEQ ID NO: 71 - protein, 17 aa, Artificial Sequence
  HCDR2
SIVPIFGTINNAQKFQG

SEQ ID NO: 72 - protein, 15 aa, Artificial Sequence
  HCDR3
DNTMVRGVDYYYMDV

SEQ ID NO: 73 - protein, 121 aa, Artificial Sequence
  Heavy chain variable region
EVQLVQSGAEVKKPGSSVKVSCKASGGIFSTYAISWVRQAPGQGLEWMGGIIPIFDTPNYAQKFQGRVTITADKSTSTAYMDLSSLRSEDTAVYYCAKNVRGYSAYDLDYWGQGTLVTVSS

SEQ ID NO: 74 - protein, 5 aa, Artificial Sequence
  HCDR1
TYAIS

SEQ ID NO: 75 - protein, 17 aa, Artificial Sequence
  HCDR2
GIIPIFDTPNYAQKFQG

SEQ ID NO: 76 - protein, 12 aa, Artificial Sequence
  HCDR3
NVRGYSAYDLDY

SEQ ID NO: 77 - protein, 119 aa, Artificial Sequence
  Heavy chain variable region
EVQLVQSGAEVKNPGSSVKVSCKATGGTFNTYGTNWVRQAPGQGLEWMGGIIPIFGTANYAQKFQGRVTITADKSTTTAYMEVSSLRSEDTAVYYCARGGADMGTLDYWGQGTLVTVSS

SEQ ID NO: 78 - protein, 5 aa, Artificial Sequence
  HCDR1
TYGTN

SEQ ID NO: 79 - protein, 17 aa, Artificial Sequence
  HCDR2
GIIPIFGTANYAQKFQG

SEQ ID NO: 80 - protein, 10 aa, Artificial Sequence
  HCDR3
GGADMGTLDY

SEQ ID NO: 81 - protein, 120 aa, Artificial Sequence
  Heavy chain variable region
EVQLVQSGAEVMRPGSSVKVSCKASGGIFNTYTIIWVRQAPGQGLEWMGGIIPIFDTPNFAQKFQGRLTITADKSTNTAYMELTSLRSEDTAVYYCAREGCNHGVCYPYWGQGTLVTVSS

SEQ ID NO: 82 - protein, 5 aa, Artificial Sequence
  HCDR1
TYTII

SEQ ID NO: 83 - protein, 17 aa, Artificial Sequence
  HCDR2
GIIPIFDTPNFAQKFQG

SEQ ID NO: 84 - protein, 11 aa, Artificial Sequence
  HCDR3
EGCNHGVCYPY

SEQ ID NO: 85 - protein, 121 aa, Artificial Sequence
  Heavy chain variable region
QVQLVQSGAEVKKPGSSVKVSCKASGDTFRSYGITWVRQAPGQGLEWMGGIIPIFGTTNYAQKFQGRVTITADKSTSTVYMELSSLRSEDTAVYYCARRRGYSNPHWLDPWGQGTLVTVSS

SEQ ID NO: 86 - protein, 5 aa, Artificial Sequence
  HCDR1
SYGIT

SEQ ID NO: 87 - protein, 17 aa, Artificial Sequence
  HCDR2
GIIPIFGTTNYAQKFQG

SEQ ID NO: 88 - protein, 12 aa, Artificial Sequence
  HCDR3
RRGYSNPHWLDP

SEQ ID NO: 89 - protein, 119 aa, Artificial Sequence
  Heavy chain variable region
QVQLVQSGAEVKKPGSSVKVSCKASGGTFSTYGILWVRQAPGQGLEWMGGIIPIFGTANYAQKFQGRVTITADISTSTAYMELSSLRSEDTAVYYCARGGGNYYEFVYWGQGTLVTVSS

SEQ ID NO: 90 - protein, 5 aa, Artificial Sequence
  HCDR1
TYGIL

SEQ ID NO: 91 - protein, 10 aa, Artificial Sequence
  HCDR3
GGGNYYEFVY

SEQ ID NO: 92 - protein, 121 aa, Artificial Sequence
  Heavy chain variable region
EVQLVQSGAEVKKPGSSVRVSCKASGGTFNTYAINWVRQAPGQGLEWVGRIIPIFDTANYAQKFQGRVTISADKSTTTAYMELSSLRSEDTAVFYCAKDETGYSSSNFQHWGQGTLVTVSS

SEQ ID NO: 93 - protein, 5 aa, Artificial Sequence
  HCDR1
TYAIN

SEQ ID NO: 94 - protein, 17 aa, Artificial Sequence
  HCDR2
RIIPIFDTANYAQKFQG

SEQ ID NO: 95 - protein, 12 aa, Artificial Sequence
  HCDR3
DETGYSSSNFQH

SEQ ID NO: 96 - protein, 125 aa, Artificial Sequence
  Heavy chain variable region
QVQLVQSGSELKKPGASVKVSCKASGYTFTNYAINWVRQAPGQGLEWMGWINPNTGNPTYAQGFTGRFVFSLDTSVSTAYLQISSLKAEDTAVYYCARDRKYVTNWVFAEDFQHWGQGTLVTVSS

SEQ ID NO: 97 - protein, 122 aa, Artificial Sequence
  Heavy chain variable region
QVQLVQSGAEVKRPGSSVKVSCKASGGTFNTYSITWVRQAPGQGLEWMGGIIPVFGTSKYAQKFQDRVTITADKSTNTAYMELSSLRSEDTAVYYCARDPSFSSSSGWFDPWGQGTLVTVSS

SEQ ID NO: 98 - protein, 17 aa, Artificial Sequence
  HCDR2
GIIPVFGTSKYAQKFQD

SEQ ID NO: 99 - protein, 13 aa, Artificial Sequence
  HCDR3
DPSFSSSSGWFDP

SEQ ID NO: 100 - protein, 121 aa, Artificial Sequence
  Heavy chain variable region
QVQLVQSGAEVKKPGSSVKVSCKASGGTFNTYAINWVRQAPGQGLEWMGGIIPIFDTANYAQRFQGRVTITADKSTSTAYMELSSLRSEDTAVYFCAKDQTGYSSTLFDYWGQGTLVTVSS

SEQ ID NO: 101 - protein, 17 aa, Artificial Sequence
  HCDR2
GIIPIFDTANYAQRFQG

SEQ ID NO: 102 - protein, 12 aa, Artificial Sequence
  HCDR3
DQTGYSSTLFDY

SEQ ID NO: 103 - protein, 125 aa, Artificial Sequence; identical to SEQ ID NO: 57
  Heavy chain variable region

SEQ ID NO: 104 - protein, 121 aa, Artificial Sequence
  Heavy chain variable region
EVQLVQSGAEVKKPGSSVKVSCKASGGTFSTYAISWVRQAPGQGLEWMGWIIPIFDTGNYAQKIQGRVTITADKSTSTAYMELTSLRSEDTAVYYCARHDYTNTVDAFDIWGQGTMVTVSS

SEQ ID NO: 105 - protein, 17 aa, Artificial Sequence
  HCDR2
WIIPIFDTGNYAQKIQG

SEQ ID NO: 106 - protein, 12 aa, Artificial Sequence
  HCDR3
HDYTNTVDAFDI

SEQ ID NO: 107 - protein, 121 aa, Artificial Sequence
  Heavy chain variable region
QVQLVQSGAEVKKPGSSVKVSCKASGDTFRSYGITWVRQAPGQGLEWMGGIIPVFGTTNYAQKFQGRVTITADKSTSTVFMELNSLRSEDTAVYYCARRRGYSNPHWLDPWGQGTLVTVSS

SEQ ID NO: 108 - protein, 17 aa, Artificial Sequence
  HCDR2
GIIPVFGTTNYAQKFQG

SEQ ID NO: 109 - protein, 214 aa, Artificial Sequence
  human common light chain IGKV1-39/jk1
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 107 aa, Artificial Sequence
  common light chain variable domain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 111 - protein, 107 aa, Artificial Sequence [sequence omitted]

SEQ ID NO: 112 - protein, 98 aa, Artificial Sequence
  CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 113 - protein, 15 aa, Artificial Sequence
  hinge
EPKSCDKTHTCPPCP

SEQ ID NO: 114 - protein, 110 aa, Artificial Sequence
  CH2
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 115 - protein, 107 aa, Artificial Sequence
  CH3 with KK mutations
GQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 107 aa, Artificial Sequence
  CH3 with DE mutations
GQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 253 aa, Artificial Sequence
  CD137
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDWCGPSPADLSPGASSVTPPAPAREPGHSPQIISFFLALTSTALLFLLFFLTLRFSWKRGRKKLLYIFKQPFMRPVQTTQEEDGCSCRFPEEEEGGCEL

(19) United States
(12) Patent Application Publication
(10) Pub. No.: US 2024/0052050 A1
(43) Pub. Date: Feb. 15, 2024

(54) MULTISPECIFIC ANTIBODIES FOR THE TREATMENT OF CANCER

(71) Applicant: MERUS N.V., Utrecht (NL)

(72) Inventors: Pieter Fokko VAN LOO, Utrecht (NL); Ernesto Isaac WASSERMAN, Utrecht (NL); Cornelis Jacob Johannes George BOL, Utrecht (NL); Gianluca LAUS, Utrecht (NL); Leonardo Andres SIRULNIK, Utrecht (NL)

(21) Appl. No.: 18/257,804

(22) PCT Filed: Dec. 3, 2021
(86) PCT No.: PCT/EP2021/084261

§ 371 Date: Jun. 15, 2023

(30) Foreign Application Priority Data
Dec. 16, 2020 (NL) 2027131

(51) Int. Cl. C07K 16/28; A61P 35/00

(52) U.S. Cl. CPC C07K 16/2878; A61P 35/00; C07K 16/2827; C07K 2317/31; A61K 2039/545

(57) ABSTRACT

The invention is relates to multispecific antibodies that comprise an antigen binding site that binds an extracellular part of CD137 and an antigen binding site that binds an extracellular part of a second membrane protein for use in a method of treatment of a cancer in a subject in need thereof. The invention further relates to such multispecific antibodies and methods and other aspects related thereto.

### TECHNICAL FIELD

The invention relates to the field of binding molecules. In particular it relates to the field of therapeutic binding molecules for the treatment of diseases involving aberrant cells, in particular cancer cells. In particular it relates to multispecific antibodies that bind an extracellular part of two or more different membrane associated proteins and thereby modulate a biological activity expressed by a cell.

### BACKGROUND

Cancer is still a major cause of morbidity and death in the world, in spite of the many advances that have been made in the treatment of the disease and the increased knowledge of the molecular events that lead to cancer.

Traditionally, most cancer drug discovery has focused on agents that block essential cell functions and kill dividing cells. However, in cases of advanced cancer, no matter how aggressively applied, even to the point where patients suffer life-threatening side-effects from the treatment, chemotherapy rarely results in a complete cure. In most cases, the tumors in the patients stop growing or temporarily shrink (referred to as remission) only to start proliferating again, sometimes more rapidly (referred to as relapse), and become increasingly more difficult to treat. More recently the focus of cancer drug development has moved away from broadly cytotoxic chemotherapy to targeted cytostatic therapies with less toxicity. Treatment of advanced cancer has been validated clinically in leukemia and some other cancers. However, in a majority of carcinomas, targeted approaches are still proving not to be effective enough to completely abolish cancer in the majority of the patients.

Targeting of cancers has been achieved using a variety of different methods including for instance small molecules directed towards signaling proteins on which the cancer depends for survival and/or growth; vaccines with tumor specific proteins; cell therapies with immune cells that actively kill tumor cells, and antibodies that target cytotoxic molecules to the tumor; interfere with signaling and/or that (re)direct the immune system of the host to the tumor cells.

The (re)direction of the immune system can be accomplished in several ways. One way is by activating T-cell costimulatory molecules such as the tumor-necrosis factor receptor superfamily, including CD137 (4-1BB, TNFRSF9). Activation of CD137 leads to increased T-cell proliferation, cytokine production and prolonged CD8⁺ T-cell survival. Another way is by blocking the negative signals induced by molecules involved in the immune checkpoint, such as cytotoxic T-lymphocyte-associated antigen 4 (CTLA-4), programmed cell-death (PD-1) expressed on T-cells or its cognate ligand programmed cell death 1 ligand 1 (PD-L1) which can be expressed on tumor cells. Upon binding of PD-L1 to PD-1, signal transduction results in the attenuation of T-cell receptor (TCR) signaling and T-cell exhaustion. This is a mechanism used by tumors in order to evade and/or suppress the immune system.

This immune suppression can be blocked by immune checkpoint inhibitor therapies (ICIs) such as antagonistic antibodies against PD-1 or PD-L1. ICI treatment has demonstrated remarkably durable responses in a subset of cancer patients that are correlated with activated CD8⁺ T cell infiltration and proliferation. Combinations of ICIs (e.g. anti-PD-1 and anti-CTLA-4) have been shown to further enhance efficacy, however at the cost of toxicity, as the majority of patients experience grade 3 or 4 treatment-related adverse events.

Dual targeting of the PD-1/PD-L1 axis and CD137 may be beneficial in optimally engaging specific anti-tumor immunity. Currently, the two most advanced therapeutic CD137 agonist antibodies in clinical testing are urelumab (IgG4) and utomilumab (IgG2). Development of urelumab has been halted as a consequence of dose-dependent hepatitis resulting from systemic activation of the CD137 pathway in patients. Safe administration of urelumab required a reduced dose; 0.1 mg/kg was chosen for combination studies with PD-1 inhibitors. Utomilumab is better tolerated by patients but has modest anti-tumor activity as a monotherapy and no clear synergy with PD-1 blockade in combination therapy.

There thus remains a need to provide more and better options for health care professionals to treat cancer, in particular any solid tumor harboring Microsatellite Instability-High (MSI-high or MSI-H) alterations, cervical cancer, endometrial cancer, lung cancer, brain cancer, and breast cancer.

### SUMMARY

The present disclosure provides means and methods for (re)directing immune system components in the treatment of cancer, in particular any solid tumor harboring MSI-high alterations, cervical cancer, endometrial cancer, lung cancer, brain cancer, and breast cancer.

In certain embodiments, the present disclosure provides a multispecific antibody that comprises an antigen binding site that binds an extracellular part of CD137 and an antigen binding site that binds an extracellular part of a second membrane protein for use in a method of treatment of cancer in a subject in need thereof, wherein the multispecific antibody is administered in a dose of between 25-300 mg, preferably between 25-150 or mg 25-100 mg, more preferably between 50-100 mg.

In certain embodiments, the present disclosure provides a multispecific antibody that comprises an antigen binding site that binds an extracellular part of CD137 and an antigen binding site that binds an extracellular part of a second membrane protein for use in a method of treatment of cancer in a subject in need thereof, wherein the multispecific antibody is administered in a dose of between 25-300 mg, or between 25-150, or mg 25-100 mg, or between 25-75 mg, or between 25-50 mg.

In certain embodiments, the present disclosure provides a multispecific antibody that comprises an antigen binding site that binds an extracellular part of CD137 and an antigen binding site that binds an extracellular part of a second membrane protein for use in a method of treatment of cancer in a subject in need thereof, wherein the cancer is selected from:
any solid tumor harboring MSI-high alterations;
endometrial cancer, in particular MSI-high endometrial cancer;
lung cancer, in particular non-small cell lung cancer (NSCLC), more in particular NSCLC with high expression of PD-L1; and
breast cancer, in particular metastatic breast cancer and triple negative breast cancer (TNBC).

In certain embodiments, the present disclosure provides a multispecific antibody that comprises an antigen binding site that binds an extracellular part of CD137 and an antigen binding site that binds an extracellular part of a second membrane protein for use in a method of treatment of cancer in a subject in need thereof, wherein the cancer is selected from:
any solid tumor harboring MSI-high alterations;
cervical cancer, such as PD-L1 positive cervical cancer or cervical cancer with high expression of PD-L1;
endometrial cancer, such as MSI-high endometrial cancer;
lung cancer, such as non-small cell lung cancer (NSCLC), PD-L1 positive NSCLC, or NSCLC with high expression of PD-L1;
brain cancer, such as glioblastoma; and
breast cancer, such as metastatic breast cancer or triple negative breast cancer (TNBC).

In certain embodiments, the present disclosure provides a multispecific antibody that comprises an antigen binding site that binds an extracellular part of CD137 and an antigen binding site that binds an extracellular part of a second membrane protein for use in a method of treatment of cancer in a subject in need thereof, wherein the cancer is selected from:
any solid tumor harboring MSI-high alterations;
endometrial cancer, in particular MSI-high endometrial cancer;
lung cancer, in particular non-small cell lung cancer (NSCLC), more in particular NSCLC with high expression of PD-L1; and
breast cancer, in particular metastatic breast cancer and triple negative breast cancer (TNBC), and
wherein the multispecific antibody is administered in a dose of between 10-1200 mg, preferably between 25-600 or mg 25-300 mg, more preferably between 25-150 mg or 25-100 mg, most preferably between 50-100 mg.

In certain embodiments, the present disclosure provides a multispecific antibody that comprises an antigen binding site that binds an extracellular part of CD137 and an antigen binding site that binds an extracellular part of a second membrane protein for use in a method of treatment of cancer in a subject in need thereof, wherein the cancer is selected from:
any solid tumor harboring MSI-high alterations;
cervical cancer, such as PD-L1 positive cervical cancer or cervical cancer with high expression of PD-L1;
endometrial cancer, such as MSI-high endometrial cancer;
lung cancer, such as non-small cell lung cancer (NSCLC), PD-L1 positive NSCLC, or NSCLC with high expression of PD-L1;
brain cancer, such as glioblastoma; and
breast cancer, such as metastatic breast cancer or triple negative breast cancer (TNBC), and
wherein the multispecific antibody is administered in a dose of between 10-1200 mg, or between 25-600 mg, or between 25-300 mg, or between 25-150 mg, or between 25-100 mg, or between 25-75 mg, or between 25-50 mg.

In certain embodiments, the present disclosure provides a method of treating cancer in a subject in need thereof, the method comprising administering between 25-300 mg, preferably between 25-150 or mg 25-100 mg, more preferably between 50-100 mg, of a multispecific antibody that comprises an antigen binding site that binds an extracellular part of CD137 and an antigen binding site that binds an extracellular part of a second membrane protein to the subject.

In certain embodiments, the present disclosure provides a method of treating cancer in a subject in need thereof, the method comprising administering between 25-300 mg, or between 25-150 mg, or between 25-100 mg, or between 25-75 mg, or between 25-50 mg, of a multispecific antibody that comprises an antigen binding site that binds an extracellular part of CD137 and an antigen binding site that binds an extracellular part of a second membrane protein to the subject.

In certain embodiments, the present disclosure provides a method of treating cancer in a subject in need thereof, the method comprising administering a multispecific antibody that comprises an antigen binding site that can bind an extracellular part of CD137 and an antigen binding site that can bind an extracellular part of a second membrane protein to a patient having cancer wherein the cancer is selected from:
any solid tumor harboring MSI-high alterations;
endometrial cancer, in particular MSI-high endometrial cancer;
lung cancer, in particular non-small cell lung cancer (NSCLC), more in particular NSCLC with high expression of PD-L1; and
breast cancer, in particular metastatic breast cancer or triple negative breast cancer (TNBC).

In certain embodiments, the present disclosure provides a method of treating cancer in a subject in need thereof, the method comprising administering a multispecific antibody that comprises an antigen binding site that can bind an extracellular part of CD137 and an antigen binding site that can bind an extracellular part of a second membrane protein to a patient having cancer wherein the cancer is selected from:
any solid tumor harboring MSI-high alterations;
cervical cancer, such as PD-L1 positive cervical cancer or cervical cancer with high expression of PD-L1;
endometrial cancer, such as MSI-high endometrial cancer;
lung cancer, such as non-small cell lung cancer (NSCLC), or PD-L1 positive NSCLC, or NSCLC with high expression of PD-L1;
brain cancer, such as glioblastoma; and
breast cancer, such as metastatic breast cancer or triple negative breast cancer (TNBC).

In certain embodiments, the present disclosure provides a method of treating cancer in a subject in need thereof, the method comprising administering between 10-1200 mg, preferably between 25-600 or mg 25-300 mg, more preferably between 25-150 mg or 25-100 mg, most preferably between 50-100 mg, of a multispecific antibody that comprises an antigen binding site that binds an extracellular part of CD137 and an antigen binding site that binds an extracellular part of a second membrane protein to a subject having cancer, wherein the cancer is selected from:
any solid tumor harboring MSI-high alterations;
endometrial cancer, in particular MSI-high endometrial cancer;
lung cancer, in particular non-small cell lung cancer (NSCLC), more in particular NSCLC with high expression of PD-L1; and
breast cancer, in particular metastatic breast cancer or triple negative breast cancer (TNBC).

In certain embodiments, the present disclosure provides a method of treating cancer in a subject in need thereof, the method comprising administering between 10-1200 mg, or between 25-600 mg, or between 25-300 mg, or between 25-150 mg, or between 25-100 mg, or between 25-75 mg, or between 25-50 mg, of a multispecific antibody that comprises an antigen binding site that binds an extracellular part of CD137 and an antigen binding site that binds an extracellular part of a second membrane protein to a subject having cancer, wherein the cancer is selected from:
any solid tumor harboring MSI-high alterations;
cervical cancer, such as PD-L1 positive cervical cancer or cervical cancer with high expression of PD-L1;
endometrial cancer, such as MSI-high endometrial cancer;
lung cancer, such as non-small cell lung cancer (NSCLC), or PD-L1 positive NSCLC, or NSCLC with high expression of PD-L1;
brain cancer, such as glioblastoma; and
breast cancer, such as metastatic breast cancer or triple negative breast cancer (TNBC).

### BRIEF DESCRIPTION OF THE FIGURES

FIG. 1. Ex vivo activity of an exemplary multispecific antibody that binds CD137 and PD-L1. Upper panel shows the relative proportion of T cell subsets in different segments and the percentage of CD3⁺ cells in the center of the donut in single cell suspensions derived from five human endometrial tumors as determined by flow cytometry. Bottom panel shows IFNγ levels in the supernatant of each tumor explant culture after 6 days of incubation with 1) a negative control antibody; 2) a multispecific antibody binding CD137 and PD-L1; 3) urelumab analog; 4) atezolizumab analog; 5) a combination of urelumab and atezolizumab analogs, and soluble anti-CD3; ND=not done, * p<0.01; ** p<0.0001 as determined by one way ANOVA and Tukey's test.

FIG. 2. In vivo activity of an exemplary multispecific antibody that binds CD137 and PD-L1. A) Ly95 T cells expressing an NY-ESO-specific TCR were transferred to NSG mice bearing A549 tumors expressing NY-ESO antigen and human PD-L1 and treated with 1) Ly95 cells only; 2) Ly95 cells plus a multispecific antibody binding CD137 and PD-L1; 3) Ly95 cells plus urelumab analog; 4) Ly95 cells plus atezolizumab analog; 5) Ly95 cells plus a combination of urelumab and atezolizumab analogs, end point data are shown, (n=7); B) Stacked histogram showing the percentage of human CD3⁺ lymphocytes in total live cell population in blood after red blood cell (RBC) lysis (open bar) and tumor (solid bar) derived from each of the treatment groups in A); C) Proportion of NY-ESO-1-specific T cells in tumors derived from each of the treatment groups in A), expressed as a percentage of CD3⁺ tumor-infiltrating lymphocytes (TILs), (n=7, error bars are SEM); * p<0.05 (multispecific antibody compared to Ly95) as determined by one way-ANOVA and Tukey's test.

FIG. 3. In vivo activity of an exemplary multispecific antibody that binds CD137 and PD-L1. A) Tumor volume in individual human CD34⁺ engrafted NSG mice following subcutaneous inoculation with MDA-MB-231 cells and treatment with 5 mg/kg or 0.5 mg/kg multispecific antibody or 5 mg/kg reference antibody, once every 5 days (n=9 mice per group). Treatment groups: a) negative control IgG; b) multispecific antibody; c) urelumab analog; d) atezolizumab; e) atezolizumab+urelumab analog; and f) pembrolizumab. Tumor growth and moment of death are indicated for the individual mice per group. B) Proportion of CD8⁺ (upper panel) and CD4⁺ (middle panel) tumor-infiltrating lymphocytes (TILs) in tumors derived from each of the treatment groups, expressed as a percentage of the total population of tumor cells; and proportion of PD-L1⁺ monocytes (lower panel) in tumors derived from each of the treatment groups, expressed as a percentage of all monocytes (CD11b+ cells); n=9, error bars are SEM; * p<0.05 as determined by one way-ANOVA and Tukey's test.

FIG. 4. The percentage weight change in individual human CD34+ engrafted NSG mice following subcutaneous inoculation with MDA-MB-231 cells and the following treatments (n=9 mice per group): a) negative control; b) multispecific antibody; c) urelumab analog; d) atezolizumab; e) urelumab analog and atezolizumab; f) pembrolizumab.

### DETAILED DESCRIPTION

In certain embodiments, the present disclosure provides a multispecific antibody that comprises an antigen binding site that binds an extracellular part of CD137 and an antigen binding site that binds an extracellular part of a second membrane protein for use in a method of treatment of cancer in a subject in need thereof, wherein the multispecific antibody is administered in a dose of between 25-300 mg, preferably between 25-150 mg or 25-100 mg, more preferably between 25-75 mg. In certain embodiments, the multispecific antibody is administered in a dose of between 50-100 mg or 75-125 mg. In certain embodiments, the present disclosure provides a multispecific antibody that comprises an antigen binding site that binds an extracellular part of CD137 and an antigen binding site that binds an extracellular part of a second membrane protein for use in a method of treatment of cancer in a subject in need thereof, wherein the multispecific antibody is administered in a dose of between 25-300 mg, or between 25-150 mg, or between 25-100 mg, or between 25-75 mg or 25-50 mg. In certain embodiments, the multispecific antibody is administered in a dose of between 50-100 mg or 75-125 mg. In certain embodiments, the multispecific antibody is administered in a flat dose of between 25-50 mg. In certain embodiments, the multispecific antibody is administered in a dose of 25, 30, 35, 40, 45, or 50 mg. In certain embodiments, the multispecific antibody is administered in a flat dose of 25, 30, 35, 40, 45, or 50 mg.

In certain embodiments, the present disclosure provides a method of treating cancer in a subject in need thereof, the method comprising administering between 25-300 mg, preferably between 25-150 mg or 25-100 mg, more preferably between 25-75 mg, of a multispecific antibody that comprises an antigen binding site that binds an extracellular part of CD137 and an antigen binding site that binds an extracellular part of a second membrane protein to the subject. In certain embodiments, the present disclosure provides a method of treating cancer in a subject in need thereof, the method comprising administering between 25-300 mg, or between 25-150 mg, or between 25-100 mg, or between 25-75 mg, or between 25-50 mg, of a multispecific antibody that comprises an antigen binding site that binds an extracellular part of CD137 and an antigen binding site that binds an extracellular part of a second membrane protein to the subject. In certain embodiments, the multispecific antibody is administered in a dose of between 50-100 mg or 75-125 mg. In certain embodiments, the multispecific antibody is administered in a flat dose of between 25-50 mg. In certain embodiments, the multispecific antibody is administered in a dose of 25, 30, 35, 40, 45, or 50 mg. In certain embodiments, the multispecific antibody is administered in a flat dose of 25, 30, 35, 40, 45, or 50 mg.

In certain embodiments, the cancer is selected from:
any solid tumor harboring MSI-high alterations;
endometrial cancer, in particular MSI-high endometrial cancer;
lung cancer, in particular non-small cell lung cancer (NSCLC), more in particular NSCLC with high expression of PD-L1; and
breast cancer, in particular metastatic breast cancer and triple negative breast cancer (TNBC). In certain embodiments, the cancer is selected from:
any solid tumor harboring MSI-high alterations;
cervical cancer, such as PD-L1 positive cervical cancer or cervical cancer with high expression of PD-L1;
endometrial cancer, such as MSI-high endometrial cancer;
lung cancer, such as non-small cell lung cancer (NSCLC), or PD-L1 positive NSCLC, or NSCLC with high expression of PD-L1;
brain cancer, such as glioblastoma; and
breast cancer, such as metastatic breast cancer or triple negative breast cancer (TNBC). In certain embodiments, the present disclosure provides a multispecific antibody that comprises an antigen binding site that binds an extracellular part of CD137 and an antigen binding site that binds an extracellular part of a second membrane protein for use in a method of treatment of cancer in a subject in need thereof, wherein the cancer is selected from:
any solid tumor harboring MSI-high alterations;
endometrial cancer, in particular MSI-high endometrial cancer;
lung cancer, in particular non-small cell lung cancer (NSCLC), more in particular NSCLC with high expression of PD-L1; and
breast cancer, in particular metastatic breast cancer and triple negative breast cancer (TNBC), and
wherein the multispecific antibody is administered in a dose of between 10-1200 mg, preferably between 25-600 or mg 25-300 mg, more preferably between 25-150 mg or 25-100 mg, most preferably between 50-100 mg. In certain embodiments, the present disclosure provides a multispecific antibody that comprises an antigen binding site that binds an extracellular part of CD137 and an antigen binding site that binds an extracellular part of a second membrane protein for use in a method of treatment of cancer in a subject in need thereof, wherein the cancer is selected from:
any solid tumor harboring MSI-high alterations;
cervical cancer, such as PD-L1 positive cervical cancer or cervical cancer with high expression of PD-L1;
endometrial cancer, such as MSI-high endometrial cancer;
lung cancer, such as non-small cell lung cancer (NSCLC), or PD-L1 positive NSCLC, or NSCLC with high expression of PD-L1;
brain cancer, such as glioblastoma; and
breast cancer, such as metastatic breast cancer or triple negative breast cancer (TNBC), and
wherein the multispecific antibody is administered in a dose of between 10-1200 mg, or between 25-600 mg, or between 25-300 mg, or between 25-150 mg, or between 25-100 mg, or between 25-75 mg, or between 25-50 mg. In certain embodiments, the multispecific antibody is administered in a flat dose of between 25-50 mg. In certain embodiments, the multispecific antibody is administered in a flat dose of between 25-50 mg. In certain embodiments, the multispecific antibody is administered in a dose of 25, 30, 35, 40, 45, or 50 mg. In certain embodiments, the multispecific antibody is administered in a flat dose of 25, 30, 35, 40, 45, or 50 mg.

In certain embodiments, the present disclosure provides a method of treating cancer in a subject in need thereof, the method comprising administering between 10-1200 mg, preferably between 25-600 or mg 25-300 mg, more preferably between 25-150 mg or 25-100 mg, most preferably between 50-100 mg, of a multispecific antibody that comprises an antigen binding site that binds an extracellular part of CD137 and an antigen binding site that binds an extracellular part of a second membrane protein to a subject having cancer, wherein the cancer is selected from:
any solid tumor harboring MSI-high alterations;
endometrial cancer, in particular MSI-high endometrial cancer;
lung cancer, in particular non-small cell lung cancer (NSCLC), more in particular NSCLC with high expression of PD-L1; and
breast cancer, in particular metastatic breast cancer or triple negative breast cancer (TNBC). In certain embodiments, the present disclosure provides a method of treating cancer in a subject in need thereof, the method comprising administering between 10-1200 mg, or between 25-600 mg, or between 25-300 mg, or between 25-150 mg, or between 25-100 mg, or between 25-75 mg, or between 25-50 mg, of a multispecific antibody that comprises an antigen binding site that binds an extracellular part of CD137 and an antigen binding site that binds an extracellular part of a second membrane protein to a subject having cancer, wherein the cancer is selected from:
any solid tumor harboring MSI-high alterations;
cervical cancer, such as PD-L1 positive cervical cancer or cervical cancer with high expression of PD-L1;
endometrial cancer, such as MSI-high endometrial cancer;
lung cancer, such as non-small cell lung cancer (NSCLC), or PD-L1 positive NSCLC, or NSCLC with high expression of PD-L1;
brain cancer, such as glioblastoma; and
breast cancer, such as metastatic breast cancer or triple negative breast cancer (TNBC). In certain embodiments, the multispecific antibody is administered in a flat dose of between 25-50 mg. In certain embodiments, the multispecific antibody is administered in a dose of 25, 30, 35, 40, 45, or 50 mg. In certain embodiments, the multispecific antibody is administered in a flat dose of 25, 30, 35, 40, 45, or 50 mg.

CD137 can be expressed by activated T-cells. It is also found on other cells such as dendritic cells, natural killer cells, granulocytes and cells of the blood vessel wall at sites of inflammation. The protein is known for its costimulatory activity for activation of T-cells. CD137 is known under a number of different names such as: TNFRSF9; TNF Receptor Superfamily Member 9; Tumor Necrosis Factor Receptor Superfamily Member 9; T-Cell Antigen 4-1BB Homolog; 4-1BB Ligand Receptor; T-Cell Antigen ILA; CD137 Antigen; CDw137; ILA; Interleukin-Activated Receptor, Homolog Of Mouse Ly63; Induced By Lymphocyte Activation (ILA); Homolog Of Mouse 4-1BB; Receptor Protein 4-1BB; T Cell Antigen ILA; and 4-1BB. External Ids for CD137 are HGNC: 11924; Entrez Gene: 3604; Ensembl: ENSG00000049249; OMIM: 602250; and UniProtKB: Q07011. CD137 is an inducible receptor most commonly upregulated on activated CD8+ T cells. CD137 signaling enhances T cell function by activating NF-κB [Arch et al, 1998]. Other cell immune cell types including CD4+ T cells, monocytes, B cells, dendritic cell (DC) subpopulations and granulocytes and NK cells can express CD137 at various levels [Shao et al, 2011]. In monocytes, CD137 is inducible by activation with lipopolysaccharide (LPS) and IL-1b. In B lymphocytes, CD137 expression is induced by antibodies against cell-surface immunoglobulin and by transformation with EBV. In DCs, CD137 ligation induces their maturation through upregulation of B7 co-stimulatory molecules (CD80 and CD86), in addition to enhancing their production of inflammatory cytokines (IL-6 and IL-12) and their survival [Makkouk et al, 2015]. The natural function of CD137 ligation on neutrophils is the increment of phagocytosis of bacterial and parasitic infections. In addition ligation of CD137 blocks the anti-apoptosis signals mediated by the IL-3/IL-5/GM-CSF receptors in neutrophils and eosinophils in vitro, thereby preventing granulocyte accumulation [Simon, 2001; Vinay et al, 2011]. In non-lymphoid cells such as chondrocytes, endothelial cells and tumor cells CD137 expression is driven by cytokine stimulation such as IL-1b for chondrocytes, the inflammatory cytokines TNFalpha/IFNγ/IL-1b for endothelial cells and IFNγ for tumor cells. The ligand that stimulates CD137 (CD137L) is expressed on activated antigen presenting cells. CD137 exists in the membrane as monomers and dimers [Pollok et al, 1993].

The B7 family comprises a number of structurally related, cell-surface proteins, which bind to receptors on lymphocytes that regulate immune responses. Activation of lymphocytes is initiated by engagement of cell-surface, antigen-specific T-cell receptors or B-cell receptors. Additional signals delivered simultaneously by B7 ligands further determine the immune response of these cells. These so-called ‘costimulatory’ or ‘coinhibitory’ signals are delivered by B7 family members through the CD28 family of receptors on lymphocytes. Binding of B7-family members with costimulatory receptors augments immune responses, and binding with coinhibitory receptors attenuates immune responses. Presently the following members are believed to be part of this family: B7.1 (CD80), B7.2 (CD86), inducible costimulator ligand (ICOS-L), programmed death-1 ligand (PD-L1), programmed death-2 ligand (PD-L2), B7-H3 (CD276), B7-H4, B7-H5, B7-H6 and B7-H7. B7 family members are expressed in lymphoid and non-lymphoid tissues. Effects of members on regulating immune responses are shown in the development of immunodeficiency and autoimmune diseases in mice with mutations in B7-family genes. Manipulation of the signals delivered by B7 ligands has shown potential in the treatment of autoimmunity, inflammatory diseases and cancer.

PD-L1 is a type 1 transmembrane protein that plays a role in suppressing an immune response during particular events such as pregnancy, tissue allografts, autoimmune disease and other disease states such as hepatitis. PD-L1 is expressed in various types of cancers, especially in NSCLC (Boland et al., 2013; Velcheti et al., 2014), melanoma, renal cell carcinoma, gastric cancer, hepatocellular as well as various leukemias and multiple myeloma (Bernstein et al., 2014; Thompson et al., 2005). PD-L1 is present in the cytoplasm and plasma membrane of cancer cells, but not all cancers or all cells within a tumor express PD-L1 (Dong et al., 2002). Multiple tumor microenvironment cells contribute to immune suppression by upregulating PD-L1 expression. This effect is called “adaptive immune resistance”, because the tumor protects itself by inducing PD-L1 in response to IFN-γ produced by activated T cells (Sharma et al., 2017). PD-L1 can also be regulated by oncogenes, this mechanism is known as inherent immune resistance (Akbay et al., 2013). Within the tumor microenvironment, PD-L1 is also expressed on myeloid cells and activated T cells (Tumeh et al., 2014). The expression of PD-L1 is induced by multiple proinflammatory molecules, including types I and II IFN-γ, TNF-α, LPS, GM-CSF and VEGF, as well as the cytokines IL-10 and IL-4, with IFN-γ being the most potent inducer (Sznol and Chen, 2013).

Programmed Cell Death 1 protein (PD-1) is a cell surface receptor that belongs to the CD28 family of receptors and is expressed on T cells and pro-B cells. PD-1 is presently known to bind two ligands, PD-L1 and PD-L2. PD-1, functioning as an immune checkpoint, plays an important role in down regulating the immune system by inhibiting the activation of T-cells, which in turn reduces autoimmunity and promotes self-tolerance. The inhibitory effect of PD-1 is thought to be accomplished through a dual mechanism of promoting apoptosis (programmed cell death) in antigen specific T-cells in lymph nodes while simultaneously reducing apoptosis in regulatory T cells (suppressor T cells). PD-1 is also known under a number of different aliases such as PDCD1; Programmed Cell Death 1; Systemic Lupus Erythematosus Susceptibility 2; Protein PD-1; HPD-1; PD1; Programmed Cell Death 1 Protein; CD279 Antigen; CD279; HPD-L; HSLE1; SLEB2; and PD-1. External Ids for PD-1 are HGNC: 8760; Entrez Gene: 5133; Ensembl: ENSG00000188389; OMIM: 600244; and UniProtKB: Q15116. New classes of drugs that block the activity of PD-1, the PD-1 inhibitors, activate the immune system to attack tumors and are therefore used with success to treat some types of cancer.

The binding of PD-L1 to PD-1 or B7.1 (CD80) transmits an inhibitory signal which reduces the proliferation of the PD-1 expressing T cells. PD-1 is thought to be able to control the accumulation of foreign antigen specific T cells through apoptosis. PD-L1 is expressed by a variety of cancer cells and the expression thereof is thought to be at least in part responsible for a dampening of an immune response against the cancer cell. PD-L1 is a member of the B7-family of protein and is known under a variety of other names such as CD274 Molecule; CD274 Antigen; B7 Homolog 1; PDCD1 Ligand 1; PDCD1LG1; PDCD1L1; B7H1; PDL1; Programmed Cell Death 1 Ligand 1; Programmed Death Ligand 1; B7-H1; and B7-H. External Ids for CD274 are HGNC: 17635; Entrez Gene: 29126; Ensembl: ENSG00000120217; OMIM: 605402; UniProtKB: Q9NZQ7.

PD-L2 is a second ligand for PD-1. Engagement of PD-1 by PD-L2 inhibits T cell receptor (TCR)-mediated proliferation and cytokine production by CD4+ T cells. At low antigen concentrations, PD-L2/PD-1 binding inhibits B7-CD28 signals. At high antigen concentrations, PD-L2/PD-1 binding reduces cytokine production. PD-L expression is up-regulated on antigen-presenting cells by interferon gamma treatment. It is expressed in some normal tissues and a variety of tumors. PD-L1 and PD-L2 are thought to have overlapping functions and regulate T cell responses. The protein is known under a number of other names such as Programmed Cell Death 1 Ligand 2; B7 Dendritic Cell Molecule; Programmed Death Ligand 2; Butyrophilin B7-DC; PDCD1 Ligand 2; PD-1 Ligand 2; PDCD1L2; B7-DC; CD273; B7DC; PDL2; PD-1-Ligand 2; CD273 Antigen; BA574F11.2; and Btdc. External Ids for PD-L2 are HGNC: 18731; Entrez Gene: 80380; Ensembl: ENSG00000197646; OMIM: 605723; and UniProtKB: Q9BQ51.

In certain embodiments, the multispecific antibody according to the use or method of the present disclosure binds to a second membrane protein that is not a member of the TNF receptor superfamily. In certain embodiments, the second membrane protein is a member of the B7 family. In certain embodiments, the second membrane protein is PD-L1 or PD-L2, preferably PD-L1.

In certain embodiments, the multispecific antibody according to the use or method of the present disclosure comprises one antigen binding site that binds to the PD-1 binding domain of PD-L1.

In certain embodiments, the multispecific antibody according to the use or method of the present disclosure binds a second membrane protein that is not to a significant extent expressed by a T-cell.

In certain embodiments, the multispecific antibody according to the use or method of the present disclosure binds a second membrane protein that is expressed on an antigen presenting cell, a tumor cell, a virus infected cell or a parasite infected cell.

In certain embodiments, the multispecific antibody according to the use or method of the present disclosure binds a second membrane protein that is a membrane protein that is present in one or more zones on the cell membrane. Preferably, the zone is a cluster, domain, micro-domain or compartment on the cell membrane, preferably an immunological synapse.

In certain embodiments, the multispecific antibody according to the use or method of the present disclosure binds a second membrane protein that is present on the cell membrane as a part of a multimeric membrane protein comprising two or more of said second membrane proteins. In certain embodiments, the second membrane protein is present on the cell membrane as a part of a homodimer or a homotrimer.

In certain embodiments, the multispecific antibody according to the use or method of the present disclosure comprises one antigen binding site that binds to the CD137L binding domain of CD137.

In certain embodiments, the multispecific antibody according to the use or method of the present disclosure comprises one antigen binding site that blocks the binding of a ligand to CD137 or binds to an extracellular ligand-blocking binding site of CD137, preferably a CD137L blocking binding site.

In certain embodiments, the multispecific antibody according to the use or method of the present disclosure comprises a variable domain that binds an extracellular part of CD137 which is defined as a variable domain that, when in a bivalent monospecific antibody format that comprises two of said variable domains that bind CD137, does not stimulate activity of CD137 on a cell or does so at a reduced level in comparison to one of said variable domain as part of a multispecific antibody having a second variable domain binding a tumor associated antigen, preferably a member of the B7 family, more preferably PD-L1.

In certain embodiments, the multispecific antibody according to the use or method of the present disclosure comprises a variable domain that binds an extracellular part of CD137 which is capable of stimulating activity of CD137 on a cell when combined in a multispecific antibody with a second variable domain which binds to PD-L1, when the multispecific antibody is in the presence of a first cell expressing CD137 and a second cell expressing PD-L1.

In certain embodiments, the multispecific antibody according to the use or method of the present disclosure is capable of binding CD137 and PD-L1 simultaneously.

A multispecific antibody according to the present disclosure that binds CD137 and an extracellular part of a second membrane protein, in particular a membrane protein that is a member of the B7 family, provides the advantage that a desired immune response can be particularly well promoted, since B7 family members deliver ‘costimulatory’ or ‘coinhibitory’ signals to lymphocytes, thereby augmenting or attenuating an immune response. Hence, by targeting a second transmembrane protein, in particular a transmembrane protein that is a member of the B7 family, it is possible to enhance stimulatory signals and/or to counteract inhibitory signals, thereby inducing or enhancing a desired immune response, for instance against aberrant cells such as cancer cells. Consequently, according to the present disclosure, the multispecific antibody is used in the treatment of cancer in a subject in need thereof when a desired immune response is elicited against aberrant cells that are found in cancer, in particular MSI-high cancers; cervical cancer; endometrial cancer, such as for instance MSI-high endometrial cancer; lung cancer, such as for instance non-small cell lung cancer (NSCLC); brain cancer, such as glioblastoma; and breast cancer, such as for instance breast adenocarcinoma and triple negative breast cancer (TNBC).

In certain embodiments, a multispecific antibody according to the present disclosure has one antigen binding site that can bind an extracellular part of CD137 and a second antigen binding site that can bind an extracellular part of a second membrane protein that is not a member of the TNF receptor superfamily but preferably a member of the B7 family, more preferably PD-L1. This provides the advantage that in cis activation of (immune) cells such as T cells expressing several different members of the TNF receptor superfamily is at least in part avoided, thereby reducing the potential adverse side effects and toxicity due to nonspecific T cell activation. Prior art approaches may lead to T cell activation in cis, meaning in the absence of a second target, and may involve the risk of an excessive T cell response, for instance resulting in a cytokine storm. Consequently, such prior art approaches have an increased potential of adverse side effects compared to a binding molecule according to the invention having an antigen binding site that can bind CD137 and an antigen binding site that can bind an extracellular part of a second membrane protein.

In certain embodiments, the present disclosure relates to a multispecific antibody targeting both PD-L1 and CD137 to partly avoid in cis activation of (immune) cells such as T cells. In certain embodiments, the variable domain that binds an extracellular part of CD137 is a domain that, when in a bivalent monospecific antibody format that comprises two of such CD137 binding domains, does not stimulate activity of CD137 on a cell or does so at a reduced level in comparison to one of said variable domain as part of a multispecific antibody having a second variable domain binding a tumor associated antigen, preferably a member of the B7 family, more preferably PD-L1. Suitable CD137 binding arms are disclosed in WO 2018/056821.

In certain embodiments, a multispecific antibody according to the use or method of the present disclosure, may be an agonistic CD137 antibody, for example an antibody that is capable of stimulating activity of CD137. In certain embodiments, a multispecific antibody according to the use or method of the present disclosure may be an antagonistic CD137 antibody, for example an antibody that is capable of reducing activity of CD137.

In certain embodiments, a multispecific antibody according to the use or method of the present disclosure may be an agonistic B7 antibody, for example an antibody that is capable of stimulating activity of a B7 family member. In certain embodiments, a multispecific antibody according to the use or method of the present disclosure may be an antagonistic B7 antibody, for example an antibody that is capable of reducing activity of a B7 family member.

In certain embodiments, a multispecific antibody according to the use or method of the present disclosure may be an agonistic PD-L1 antibody, for example an antibody that is capable of stimulating activity of PD-L1. In certain embodiments, a multispecific antibody according to the use or method of the present disclosure may be an antagonistic PD-L1 antibody, for example an antibody that is capable of reducing activity of PD-L1.

In certain embodiments, a multispecific antibody according to the use or method of the present disclosure may stimulate CD137 activity when bound to a B7 family member. In certain embodiments, the multispecific antibody stimulates CD137 activity when bound to CD137 and PD-L1. In certain embodiments, the multispecific antibody only induces or activates CD137 signaling in the presence of PD-L1 expressing cells.

In certain embodiments, the multispecific antibody according to the use or method of the present disclosure comprises antigen binding sites consisting of one immunoglobulin variable domain that binds to CD137 and one immunoglobulin variable domain that binds to an extracellular part of a second membrane protein.

In certain embodiments, variable domains comprised by the multispecific antibody according to the use or method of the present disclosure that bind to an extracellular part of CD137 and that at least partially block the binding of CD137 ligand to CD137 are variable domains that comprise the amino acid sequence of the VH of: MF6783 (SEQ ID NO: 1); MF6861 (SEQ ID NO: 5); MF6795 (SEQ ID NO: 9); MF6808 (SEQ ID NO: 13); MF6798 (SEQ ID NO: 17); MF6754 (SEQ ID NO: 20); MF6763 (SEQ ID NO: 24); MF6744 (SEQ ID NO: 28); MF6785 (SEQ ID NO: 31); MF6825 (SEQ ID NO: 35); MF6737 (SEQ ID NO: 39); MF6749 (SEQ ID NO: 43); MF6788 (SEQ ID NO: 46); or MF6797 (SEQ ID NO: 49).

In certain embodiments, a multispecific antibody or a functional part, derivative and/or analogue thereof according to the use or method of the present disclosure that comprises a variable domain that binds to an extracellular part of CD137 comprises a heavy chain variable region with a CDR3 region that comprises the amino acid sequence of the CDR3 region of the variable heavy chain region of MF6754 (SEQ ID NO: 23); MF6763 (SEQ ID NO: 27); MF6785 (SEQ ID NO: 34); or MF6797 (SEQ ID NO: 52).

In certain embodiments, a multispecific antibody or a functional part, derivative and/or analogue thereof according to the use or method of the present disclosure that comprises a variable domain that binds to an extracellular part of CD137 comprises a heavy chain variable region with a CDR2 region that comprises the amino acid sequence of the CDR2 region of the variable heavy chain region of MF6754 (SEQ ID NO: 22); MF6763 (SEQ ID NO: 26); MF6785 (SEQ ID NO: 33); or MF6797 (SEQ ID NO: 51).

In certain embodiments, a multispecific antibody or a functional part, derivative and/or analogue thereof according to the use or method of the present disclosure that comprises a variable domain that binds to an extracellular part of CD137 comprises a heavy chain variable region with a CDR1 region that comprises the amino acid sequence of the CDR1 region of the variable heavy chain region of MF6754 (SEQ ID NO: 21); MF6763 (SEQ ID NO: 25); MF6785 (SEQ ID NO: 32); or MF6797 (SEQ ID NO: 50).

In certain embodiments, a multispecific antibody or a functional part, derivative and/or analogue thereof according to the use or method of the present disclosure that comprises a variable domain that binds to an extracellular part of CD137 comprises a heavy chain variable region with a CDR1, CDR2 and CDR3 region that comprises the amino acid sequence of the CDR1, CDR2 and CDR3 of a variable heavy chain region of one of the VH presented for MF6754; MF6763; MF6785; or MF6797. The CDR1, CDR2 and CDR3 sequences are preferably selected from the same VH region.

In certain embodiments, a multispecific antibody or a functional part, derivative and/or analogue thereof according to the use or method of the present disclosure that comprises a variable domain that binds to an extracellular part of CD137 comprises the amino acid sequence of the variable heavy chain region of MF6754; MF6763; MF6785; or MF6797 having at most 15, preferably 0, 1, 2, 3, 4, 5, 6, 7, 8, 9 or 10 and preferably having 0, 1, 2, 3, 4 or 5 amino acid insertions, deletions, substitutions or a combination thereof with respect to the amino acid sequence of the VH of the indicated MF. In certain embodiments, the amino acid sequence of the variable heavy chain region has at most 5, preferably 0, 1, 2, 3, or 4; more preferably 0, 1, 2, or 3, more preferably 0, 1 or 2; most preferably 0 or 1, amino acid insertions, deletions, substitutions or a combination thereof with respect to the amino acid sequence of the VH of the indicated MF. In certain embodiments, the amino acid insertion(s), deletion(s), substitution(s) or a combination thereof, if any, are not in the amino acid sequence of the CDR regions.

In certain embodiments, the variable domain that binds an extracellular part of CD137 or a functional part, derivative and/or analogue thereof comprised by the multispecific antibody according to the use or method of the present disclosure comprises a VH region with the amino acid sequence of the CDR3 or the amino acid sequence of the CDR1, CDR2 and CDR3 of one of the VH of MF6754; MF6763; MF6785; or MF6797. In certain embodiments, the variable domain that binds an extracellular part of CD137 comprises a VH region with the amino acid sequence of the VH of MF6754 (SEQ ID NO: 20); MF6763 (SEQ ID NO: 24); MF6785 (SEQ ID NO: 31); or MF6797 (SEQ ID NO: 49) having at most 15, preferably 0, 1, 2, 3, 4, 5, 6, 7, 8, 9 or 10 and preferably having 0, 1, 2, 3, 4 or 5 amino acid insertions, deletions, substitutions or a combination thereof with respect to the amino acid sequence of the VH of the indicated MF. In certain embodiments, the amino acid sequence of the variable heavy chain region has at most 5, preferably 0, 1, 2, 3, or 4; more preferably 0, 1, 2, or 3, more preferably 0, 1 or 2; most preferably 0 or 1, amino acid insertions, deletions, substitutions or a combination thereof with respect to the amino acid sequence of the VH of the indicated MF. In certain embodiments, the amino acid insertion(s), deletion(s), substitution(s) or a combination thereof, if any, are not in the amino acid sequence of the CDR regions.

In certain embodiments, variable domains comprised by the multispecific antibody or a functional part, derivative and/or analogue thereof according to the use or method of the present disclosure that bind an extracellular part of PD-L1 and that block the binding of PD1 to PD-L1 are variable domains that comprise the amino acid sequence of the VH of MF5554 (SEQ ID NO: 53); MF5576 (SEQ ID NO: 57); MF5578 (SEQ ID NO: 59); MF9375 (SEQ ID NO: 62); MF9376 (SEQ ID NO: 64); MF7702 (SEQ ID NO: 67); MF5359 (SEQ ID NO: 69); MF5377 (SEQ ID NO: 73); MF5382 (SEQ ID NO: 77); MF5424 (SEQ ID NO: 81); MF5426 (SEQ ID NO: 85); MF5439 (SEQ ID NO: 89); MF5442 (SEQ ID NO: 92); MF5553 (SEQ ID NO: 96); MF5557 (SEQ ID NO: 97); MF5561 (SEQ ID NO: 100); MF5576 (SEQ ID NO: 103); MF5594 (SEQ ID NO: 104); or MF5708 (SEQ ID NO: 107).

In certain embodiments, a multispecific antibody or a functional part, derivative and/or analogue thereof according to the use or method of the present disclosure that comprises a variable domain that binds to an extracellular part of PD-L1 comprises a heavy chain variable region with a CDR3 region that comprises the amino acid sequence of the CDR3 region of the variable heavy chain region of MF5554 (SEQ ID NO: 56); MF5576 (SEQ ID NO: 58); MF5578 (SEQ ID NO: 61); MF9375 (SEQ ID NO: 56); MF9376 (SEQ ID NO: 56); MF7702 (SEQ ID NO: 56); MF5424 (SEQ ID NO: 84); MF5561 (SEQ ID NO: 102); MF5439 (SEQ ID NO: 91); MF5553 (SEQ ID NO: 56); MF5594 (SEQ ID NO: 106); MF5426 (SEQ ID NO: 88); or MF5442 (SEQ ID NO: 95).

In certain embodiments, a multispecific antibody or a functional part, derivative and/or analogue thereof according to the use or method of the present disclosure comprises a heavy chain variable region that binds an extracellular part of PD-L1 comprising a CDR3 region having an amino acid sequence as set forth in SEQ ID NO: 56; SEQ ID NO: 58; SEQ ID NO: 61; SEQ ID NO: 84; SEQ ID NO: 88; SEQ ID NO: 91; SEQ ID NO: 95; SEQ ID NO: 102; or SEQ ID NO: 106, preferably SEQ ID NO: 56; SEQ ID NO: 91; SEQ ID NO: 95; or SEQ ID NO: 102, or variants thereof.

In certain embodiments, a multispecific antibody or a functional part, derivative and/or analogue thereof according to the use or method of the present disclosure that comprises a variable domain that binds to an extracellular part of PD-L1 comprises a heavy chain variable region with a CDR2 region that comprises the amino acid sequence of the CDR2 region of the variable heavy chain region of MF5554 (SEQ ID NO: 55); MF5576 (SEQ ID NO: 55); MF5578 (SEQ ID NO: 3); MF9375 (SEQ ID NO: 63); MF9376 (SEQ ID NO: 66); MF7702 (SEQ ID NO: 55); MF5424 (SEQ ID NO: 83); MF5561 (SEQ ID NO: 101); MF5439 (SEQ ID NO: 79); MF5553 (SEQ ID NO: 55); MF5594 (SEQ ID NO: 105); MF5426 (SEQ ID NO: 87); or MF5442 (SEQ ID NO: 94).

In certain embodiments, a multispecific antibody or a functional part, derivative and/or analogue thereof according to the use or method of the present disclosure comprises a variable domain that binds to an extracellular part of PD-L1, wherein the antibody comprises a heavy chain variable region that binds an extracellular part of PD-L1 comprising a CDR2 region having an amino acid sequence as set forth in SEQ ID NO: 3; SEQ ID NO: 55; SEQ ID NO: 63; SEQ ID NO: 66; SEQ ID NO: 79; SEQ ID NO: 83; SEQ ID NO: 87; SEQ ID NO: 94; SEQ ID NO: 101; or SEQ ID NO: 105, or variants thereof.

In certain embodiments, a multispecific antibody or a functional part, derivative and/or analogue thereof according to the use or method of the present disclosure that comprises a variable domain that binds to an extracellular part of PD-L1 comprises a heavy chain variable region with a CDR1 region that comprises the amino acid sequence of the CDR1 region of the variable heavy chain region of MF5554 (SEQ ID NO: 54); MF5576 (SEQ ID NO: 54); MF5578 (SEQ ID NO: 60); MF9375 (SEQ ID NO: 60); MF9376 (SEQ ID NO: 65); MF7702 (SEQ ID NO: 68); MF5424 (SEQ ID NO: 82); MF5561 (SEQ ID NO: 93); MF5439 (SEQ ID NO: 90); MF5553 (SEQ ID NO: 68); MF5594 (SEQ ID NO: 74); MF5426 (SEQ ID NO: 86); or MF5442 (SEQ ID NO: 93).

In certain embodiments, a multispecific antibody or according to the use or method of the present disclosure that comprises a variable domain that binds to an extracellular part of PD-L1 comprises a heavy chain variable region that binds an extracellular part of PD-L1 comprising a CDR1 region having an amino acid sequence as set forth in SEQ ID NO: 54; SEQ ID NO: 60; SEQ ID NO: 65; SEQ ID NO: 68; SEQ ID NO: 74; SEQ ID NO: 82; SEQ ID NO: 86; SEQ ID NO: 90; or SEQ ID NO: 93, or variants thereof.

In certain embodiments, a multispecific antibody or a functional part, derivative and/or analogue thereof according to the use or method of the present disclosure that comprises a variable domain that binds to an extracellular part of PD-L1 comprises a heavy chain variable region with a CDR1, CDR2 and CDR3 region that comprises the amino acid sequence of the CDR1, CDR2 and CDR3 of a variable heavy chain region of one of the VH presented for MF5554; MF5576; MF5578; MF9375; MF9376; MF7702; MF5424; MF5561; MF5439; MF5553; MF5594; MF5426; or MF5442. The CDR1, CDR2 and CDR3 sequences are preferably selected from the same VH region.

In certain embodiments, a multispecific antibody or a functional part, derivative and/or analogue thereof according to the use or method of the present disclosure that comprises a variable domain that binds to an extracellular part of PD-L1 comprises the amino acid sequence of the variable heavy chain region of MF5554 (SEQ ID NO: 53); MF5576 (SEQ ID NO: 57); MF5578 (SEQ ID NO: 59); MF9375 (SEQ ID NO: 62); MF9376 (SEQ ID NO: 64); MF7702 (SEQ ID NO: 67); MF5424 (SEQ ID NO: 81); MF5561 (SEQ ID NO: 100); MF5439 (SEQ ID NO: 89); MF5553 (SEQ ID NO: 96); MF5594 (SEQ ID NO: 104); MF5426 (SEQ ID NO: 85); or MF5442 (SEQ ID NO: 92), having at most 15, preferably 0, 1, 2, 3, 4, 5, 6, 7, 8, 9 or 10 and preferably having 0, 1, 2, 3, 4 or 5 amino acid insertions, deletions, substitutions or a combination thereof with respect to the amino acid sequence of the VH of the indicated MF. In certain embodiments, the amino acid sequence of the variable heavy chain region has at most 5, preferably 0, 1, 2, 3, or 4; more preferably 0, 1, 2, or 3, more preferably 0, 1 or 2; most preferably 0 or 1, amino acid insertions, deletions, substitutions or a combination thereof with respect to the amino acid sequence of the VH of the indicated MF. In certain embodiments, the amino acid insertion(s), deletion(s), substitution(s) or a combination thereof, if any, are not in the amino acid sequence of the CDR regions.

In certain embodiments, a particularly preferred combination in the multispecific antibody or functional part, derivative and/or analogue according to the use or method of the present disclosure is the combination of variable domains that comprise the indicated sequence or variant thereof of MF6797 (SEQ ID NO: 49) and MF7702 (SEQ ID NO: 67); MF6763 (SEQ ID NO: 24) and MF7702 (SEQ ID NO: 67); MF6785 (SEQ ID NO: 31) and MF7702 (SEQ ID NO: 67); MF6797 (SEQ ID NO: 49) and MF5553 (SEQ ID NO: 96); MF6763 (SEQ ID NO: 24) and MF5553 (SEQ ID NO: 96); MF6785 (SEQ ID NO: 31) and MF5553 (SEQ ID NO: 96); MF6754 (SEQ ID NO: 20) and MF5424 (SEQ ID NO: 81); MF6763 (SEQ ID NO: 24) and MF5561 (SEQ ID NO: 100); MF6785 (SEQ ID NO: 31) and MF5439 (SEQ ID NO: 89); MF6754 (SEQ ID NO: 20) and MF5553 (SEQ ID NO: 96); MF6744 (SEQ ID NO: 28) and MF5594 (SEQ ID NO: 104); or MF6783 (SEQ ID NO: 1) and MF5594 (SEQ ID NO: 104).

In certain embodiments, a multispecific antibody or a functional part, derivative and/or analogue thereof according to the use or method of the present disclosure comprises:
a CD137 binding variable domain that comprises a VH region with the amino acid sequence of the CDR3 or the amino acid sequence of the CDR1, CDR2 and CDR3 of the VH of MF6797 (SEQ ID NO: 49); and
a PD-L1 binding variable domain that comprises a VH region with the amino acid sequence of the CDR3 or the amino acid sequence of the CDR1, CDR2 and CDR3 of the VH of MF5554 (SEQ ID NO: 53); MF5576 (SEQ ID NO: 57); MF5578 (SEQ ID NO: 59); MF9375 (SEQ ID NO: 62); MF9376 (SEQ ID NO: 64); MF7702 (SEQ ID NO: 67); MF5594 (SEQ ID NO: 104); MF5424 (SEQ ID NO: 81); MF5426 (SEQ ID NO: 85); MF5553 (SEQ ID NO: 96); MF5442 (SEQ ID NO: 92); MF5561 (SEQ ID NO: 100); or MF5439 (SEQ ID NO: 89).

In certain embodiments, a multispecific antibody or a functional part, derivative and/or analogue thereof according to the use or method of the present disclosure comprises:
a CD137 binding variable domain that comprises a VH region with the amino acid sequence of the VH of MF6797 (SEQ ID NO: 49) having at most 15, preferably 0, 1, 2, 3, 4, 5, 6, 7, 8, 9 or 10 and preferably having 0, 1, 2, 3, 4 or 5 amino acid insertions, deletions, substitutions or a combination thereof with respect to the amino acid sequence of the amino acid sequence of the VH of MF6797 (SEQ ID NO: 49) and;
a PD-L1 binding variable domain that comprises a VH region with the amino acid sequence of the VH of MF5554 (SEQ ID NO: 53); MF5576 (SEQ ID NO: 57); MF5578 (SEQ ID NO: 59); MF9375 (SEQ ID NO: 62); MF9376 (SEQ ID NO: 64); MF7702 (SEQ ID NO: 67); MF5594 (SEQ ID NO: 104); MF5424 (SEQ ID NO: 81); MF5426 (SEQ ID NO: 85); MF5553 (SEQ ID NO: 96); MF5442 (SEQ ID NO: 92); MF5561 (SEQ ID NO: 100); or MF5439 (SEQ ID NO: 89), having at most 15, preferably 0, 1, 2, 3, 4, 5, 6, 7, 8, 9 or 10 and preferably having 0, 1, 2, 3, 4 or 5 amino acid insertions, deletions, substitutions or a combination thereof with respect to the amino acid sequence of the VH of the indicated MF. In certain embodiments, the amino acid sequence of the variable heavy chain regions have at most 5, preferably 0, 1, 2, 3, or 4; more preferably 0, 1, 2, or 3, more preferably 0, 1 or 2; most preferably 0 or 1, amino acid insertions, deletions, substitutions or a combination thereof with respect to the amino acid sequence of the VH of the indicated MFs.

Certain embodiments further provide a multispecific antibody or a functional part, derivative and/or analogue thereof according to the use or method of the present disclosure that comprises:
a CD137 binding variable domain that comprises a VH region with the amino acid sequence of the CDR3 region of the VH of MF6763 (SEQ ID NO: 27); and
a PD-L1 binding variable domain that comprises a VH region with the amino acid sequence of the CDR3 region of the VH of MF5442 (SEQ ID NO: 95).

Certain embodiments provide a multispecific antibody or a functional part, derivative and/or analogue thereof that comprises:
a CD137 binding variable domain that comprises a VH region with the amino acid sequence of the CDR1, CDR2 and CDR3 regions of the VH of MF6763 (SEQ ID NO: 24); and
a PD-L1 binding variable domain that comprises a VH region with the amino acid sequence of the CDR1, CDR2 and CDR3 regions of the VH of MF5442 (SEQ ID NO: 92).

Certain embodiments provide a multispecific antibody or a functional part, derivative and/or analogue thereof according to the use or method of the present disclosure that comprises:
a CD137 binding variable domain that comprises a VH region with the amino acid sequence of the VH of MF6763 (SEQ ID NO: 24) having at most 15, preferably 0, 1, 2, 3, 4, 5, 6, 7, 8, 9 or 10 and preferably having 0, 1, 2, 3, 4 or 5 amino acid insertions, deletions, substitutions or a combination thereof with respect to the amino acid sequence of the amino acid sequence of the VH of MF6763; and
a PD-L1 binding variable domain that comprises a VH region with the amino acid sequence of the VH of MF5442 (SEQ ID NO: 92) having at most 15, preferably 0, 1, 2, 3, 4, 5, 6, 7, 8, 9 or 10 and preferably having 0, 1, 2, 3, 4 or 5 amino acid insertions, deletions, substitutions or a combination thereof with respect to the amino acid sequence of the VH of MF5442. In certain embodiments, the amino acid sequence of the variable heavy chain regions have at most 5, preferably 0, 1, 2, 3, or 4; more preferably 0, 1, 2, or 3, more preferably 0, 1 or 2; most preferably 0 or 1, amino acid insertions, deletions, substitutions or a combination thereof with respect to the amino acid sequence of the VH of the indicated MFs.

Certain embodiments further provide a multispecific antibody or a functional part, derivative and/or analogue thereof according to the use or method of the present disclosure that comprises:
a CD137 binding variable domain that comprises a VH region with the amino acid sequence of the CDR3 region of the VH of MF6797 (SEQ ID NO: 52); and
a PD-L1 binding variable domain that comprises a VH region with the amino acid sequence of the CDR3 region of the VH of MF7702 (SEQ ID NO: 56).

Certain embodiments provide a multispecific antibody or a functional part, derivative and/or analogue thereof that comprises:
a CD137 binding variable domain that comprises a VH region with the amino acid sequence of the CDR1, CDR2 and CDR3 regions of the VH of MF6797 (SEQ ID NO: 49); and
a PD-L1 binding variable domain that comprises a VH region with the amino acid sequence of the CDR1, CDR2 and CDR3 regions of the VH of MF7702 (SEQ ID NO: 67).

Certain embodiments provide a multispecific antibody or a functional part, derivative and/or analogue thereof according to the use or method of the present disclosure that comprises:
a CD137 binding variable domain that comprises a VH region with the amino acid sequence of the VH of MF6797 (SEQ ID NO: 49) having at most 15, preferably 0, 1, 2, 3, 4, 5, 6, 7, 8, 9 or 10 and preferably having 0, 1, 2, 3, 4 or 5 amino acid insertions, deletions, substitutions or a combination thereof with respect to the amino acid sequence of the amino acid sequence of the VH of MF6797 and;
a PD-L1 binding variable domain that comprises a VH region with the amino acid sequence of the VH of MF7702 (SEQ ID NO: 67) having at most 15, preferably 0, 1, 2, 3, 4, 5, 6, 7, 8, 9 or 10 and preferably having 0, 1, 2, 3, 4 or 5 amino acid insertions, deletions, substitutions or a combination thereof with respect to the amino acid sequence of the VH of MF7702. In certain embodiments, the amino acid sequence of the variable heavy chain regions have at most 5, preferably 0, 1, 2, 3, or 4; more preferably 0, 1, 2, or 3, more preferably 0, 1 or 2; most preferably 0 or 1, amino acid insertions, deletions, substitutions or a combination thereof with respect to the amino acid sequence of the VH of the indicated MFs.

Certain embodiments further provide a multispecific antibody or a functional part, derivative and/or analogue thereof according to the use or method of the present disclosure that comprises:
a CD137 binding variable domain that comprises a VH region with the amino acid sequence of the CDR3 region of the VH of MF6754 (SEQ ID NO: 23); and
a PD-L1 binding variable domain that comprises a VH region with the amino acid sequence of the CDR3 region of the VH of MF5561 (SEQ ID NO: 102).

Certain embodiments provide a multispecific antibody or a functional part, derivative and/or analogue thereof that comprises:
a CD137 binding variable domain that comprises a VH region with the amino acid sequence of the CDR1, CDR2 and CDR3 regions of the VH of MF6754 (SEQ ID NO: 20); and
a PD-L1 binding variable domain that comprises a VH region with the amino acid sequence of the CDR1, CDR2 and CDR3 regions of the VH of MF5561 (SEQ ID NO: 100).

Certain embodiments provide a multispecific antibody or a functional part, derivative and/or analogue thereof according to the use or method of the present disclosure that comprises:
a CD137 binding variable domain that comprises a VH region with the amino acid sequence of the VH of MF6754 (SEQ ID NO: 20) having at most 15, preferably 0, 1, 2, 3, 4, 5, 6, 7, 8, 9 or 10 and preferably having 0, 1, 2, 3, 4 or 5 amino acid insertions, deletions, substitutions or a combination thereof with respect to the amino acid sequence of the amino acid sequence of the VH of MF6754 and;
a PD-L1 binding variable domain that comprises a VH region with the amino acid sequence of the VH of MF5561 (SEQ ID NO: 100) having at most 15, preferably 0, 1, 2, 3, 4, 5, 6, 7, 8, 9 or 10 and preferably having 0, 1, 2, 3, 4 or 5 amino acid insertions, deletions, substitutions or a combination thereof with respect to the amino acid sequence of the VH of MF5561. In certain embodiments, the amino acid sequence of the variable heavy chain regions have at most 5, preferably 0, 1, 2, 3, or 4; more preferably 0, 1, 2, or 3, more preferably 0, 1 or 2; most preferably 0 or 1, amino acid insertions, deletions, substitutions or a combination thereof with respect to the amino acid sequence of the VH of the indicated MFs.

Certain embodiments further provide a multispecific antibody or a functional part, derivative and/or analogue thereof according to the use or method of the present disclosure that comprises:
a CD137 binding variable domain that comprises a VH region with the amino acid sequence of the CDR3 region of the VH of MF6785 (SEQ ID NO: 34); and
a PD-L1 binding variable domain that comprises a VH region with the amino acid sequence of the CDR3 region of the VH of MF5439 (SEQ ID NO: 91).

Also provided is a bispecific antibody or a functional part, derivative and/or analogue thereof that comprises:
a CD137 binding variable domain that comprises a VH region with the amino acid sequence of the CDR1, CDR2 and CDR3 regions of the VH of MF6785 (SEQ ID NO: 31); and
a PD-L1 binding variable domain that comprises a VH region with the amino acid sequence of the CDR1, CDR2 and CDR3 regions of the VH of MF5439 (SEQ ID NO: 89).

Certain embodiments provide a multispecific antibody or a functional part, derivative and/or analogue thereof according to the use or method of the present disclosure that comprises:
a CD137 binding variable domain that comprises a VH region with the amino acid sequence of the VH of MF6785 (SEQ ID NO: 31) having at most 15, preferably 0, 1, 2, 3, 4, 5, 6, 7, 8, 9 or 10 and preferably having 0, 1, 2, 3, 4 or 5 amino acid insertions, deletions, substitutions or a combination thereof with respect to the amino acid sequence of the amino acid sequence of the VH of MF6785; and
a PD-L1 binding variable domain that comprises a VH region with the amino acid sequence of the VH of MF5439 (SEQ ID NO: 89) having at most 15, preferably 0, 1, 2, 3, 4, 5, 6, 7, 8, 9 or 10 and preferably having 0, 1, 2, 3, 4 or 5 amino acid insertions, deletions, substitutions or a combination thereof with respect to the amino acid sequence of the VH of MF5439. In certain embodiments, the amino acid sequence of the variable heavy chain regions have at most 5, preferably 0, 1, 2, 3, or 4; more preferably 0, 1, 2, or 3, more preferably 0, 1 or 2; most preferably 0 or 1, amino acid insertions, deletions, substitutions or a combination thereof with respect to the amino acid sequence of the VH of the indicated MFs.

Certain embodiments further provide a multispecific antibody or a functional part, derivative and/or analogue thereof according to the use or method of the present disclosure that comprises:
a CD137 binding variable domain that comprises a VH region with the amino acid sequence of the CDR3 region of the VH of MF6785 (SEQ ID NO: 34); and
a PD-L1 binding variable domain that comprises a VH region with the amino acid sequence of the CDR3 region of the VH of MF5542 (SEQ ID NO: 95).

Certain embodiments provide a multispecific antibody or a functional part, derivative and/or analogue thereof that comprises:
a CD137 binding variable domain that comprises a VH region with the amino acid sequence of the CDR1, CDR2 and CDR3 regions of the VH of MF6785 (SEQ ID NO: 31); and
a PD-L1 binding variable domain that comprises a VH region with the amino acid sequence of the CDR1, CDR2 and CDR3 regions of the VH of MF5442 (SEQ ID NO: 92).

Certain embodiments provide a multispecific antibody or a functional part, derivative and/or analogue thereof according to the use or method of the present disclosure that comprises:
a CD137 binding variable domain that comprises a VH region with the amino acid sequence of the VH of MF6785 (SEQ ID NO: 31) having at most 15, preferably 0, 1, 2, 3, 4, 5, 6, 7, 8, 9 or 10 and preferably having 0, 1, 2, 3, 4 or 5 amino acid insertions, deletions, substitutions or a combination thereof with respect to the amino acid sequence of the amino acid sequence of the VH of MF6785; and
a PD-L1 binding variable domain that comprises a VH region with the amino acid sequence of the VH of MF5442 (SEQ ID NO: 92) having at most 15, preferably 0, 1, 2, 3, 4, 5, 6, 7, 8, 9 or 10 and preferably having 0, 1, 2, 3, 4 or 5 amino acid insertions, deletions, substitutions or a combination thereof with respect to the amino acid sequence of the VH of MF5542. In certain embodiments, the amino acid sequence of the variable heavy chain regions have at most 5, preferably 0, 1, 2, 3, or 4; more preferably 0, 1, 2, or 3, more preferably 0, 1 or 2; most preferably 0 or 1, amino acid insertions, deletions, substitutions or a combination thereof with respect to the amino acid sequence of the VH of the indicated MFs.

Certain embodiment provide a multispecific antibody or a functional part, derivative and/or analogue thereof according to the use or method of the present disclosure that comprises a binding domain that binds CD137, comprising
a variable domain that comprises a CDR1 according to SEQ ID NO: 50, a CDR2 according to SEQ ID NO: 51 and a CDR3 according to SEQ ID NO: 52, each CDR1, CDR2, and/or CDR3 having 0, 1, 2, 3, 4 or 5 amino acid insertions, deletions, substitutions or a combination thereof, or
a variable domain that comprises a CDR1 according to SEQ ID NO: 40, a CDR2 according to SEQ ID NO: 41 and a CDR3 according to SEQ ID NO: 42, each CDR1, CDR2 and/or CDR3 having 0, 1, 2, 3, 4 or 5 amino acid insertions, deletions, substitutions or a combination thereof, or
a variable domain that comprises a CDR1 according to SEQ ID NO: 21, a CDR2 according to SEQ ID NO: 22 and a CDR3 according to SEQ ID NO: 23, each CDR1, CDR2 and/or CDR3 having 0, 1, 2, 3, 4 or 5 amino acid insertions, deletions, substitutions or a combination thereof, or
a variable domain that comprises a CDR1 according to SEQ ID NO: 32, a CDR2 according to SEQ ID NO: 33 and a CDR3 according to SEQ ID NO: 34, each CDR1, CDR2 and/or CDR3 having 0, 1, 2, 3, 4 or 5 amino acid insertions, deletions, substitutions or a combination thereof; and/or a binding domain that binds PD-L1, comprising
a variable domain that comprises a CDR1 according to SEQ ID NO: 68, a CDR2 according to SEQ ID NO: 55 and a CDR3 according to SEQ ID NO: 56, each CDR1, CDR2 and/or CDR3 having 0, 1, 2, 3, 4 or 5 amino acid insertions, deletions, substitutions or a combination thereof, or
a variable domain that comprises a CDR1 according to SEQ ID NO: 93, a CDR2 according to SEQ ID NO: 94 and a CDR3 according to SEQ ID NO: 95, each CDR1, CDR2 and/or CDR3 having 0, 1, 2, 3, 4 or 5 amino acid insertions, deletions, substitutions or a combination thereof, or
a variable domain that comprises a CDR1 according to SEQ ID NO: 93, a CDR2 according to SEQ ID NO: 101 and a CDR3 according to SEQ ID NO: 102, each CDR1, CDR2 and/or CDR3 having 0, 1, 2, 3, 4 or 5 amino acid insertions, deletions, substitutions or a combination thereof, or
a variable domain that comprises a CDR1 according to SEQ ID NO: 90, a CDR2 according to SEQ ID NO: 79 and a CDR3 according to SEQ ID NO: 91, each CDR1, CDR2 and/or CDR3 having 0, 1, 2, 3, 4 or 5 amino acid insertions, deletions, substitutions or a combination thereof. In certain embodiments, the amino acid sequence of the CDRs have at most 5, preferably 0, 1, 2, 3, or 4; more preferably 0, 1, 2, or 3, more preferably 0, 1 or 2; most preferably 0 or 1, amino acid insertions, deletions, substitutions or a combination thereof.

In certain embodiments, a multispecific antibody or a functional part, derivative and/or analogue thereof according to the use or method of the present disclosure comprises a variable domain that binds to an extracellular part of CD137 that blocks the binding of CD137 to CD137 ligand and a variable domain that binds to an extracellular part of PD-L1 that blocks the binding of PD-1 to PD-L1. In certain embodiments, the variable domain that binds an extracellular part of PD-L1 in this antibody or a functional part, derivative and/or analogue thereof comprises a VH region with the amino acid sequence of the CDR3 or the amino acid sequence of the CDR1, CDR2 and CDR3 of one of the VH of MF5554; MF5576; MF5578; MF9375; MF9376; MF7702; MF5424; MF5561; MF5439; MF5553; MF5594; MF5426; MF5442. In certain embodiments, the variable domain that binds an extracellular part of PD-L1 comprises a VH region with the amino acid sequence of a VH of MF5554 (SEQ ID NO: 53); MF5576 (SEQ ID NO: 57); MF5578 (SEQ ID NO: 59); MF9375 (SEQ ID NO: 62); MF9376 (SEQ ID NO: 64); MF7702 (SEQ ID NO: 67); MF5424 (SEQ ID NO: 81); MF5561 (SEQ ID NO: 100); MF5439 (SEQ ID NO: 89); MF5553 (SEQ ID NO: 96); MF5594 (SEQ ID NO: 104); MF5426 (SEQ ID NO: 85); MF5442 (SEQ ID NO: 92) having at most 15, preferably 0, 1, 2, 3, 4, 5, 6, 7, 8, 9 or 10 and preferably having 0, 1, 2, 3, 4 or 5 amino acid insertions, deletions, substitutions or a combination thereof with respect to the amino acid sequence of the VH of the indicated MF. In certain embodiments, the amino acid sequence of the variable heavy chain region has at most 5, preferably 0, 1, 2, 3, or 4; more preferably 0, 1, 2, or 3, more preferably 0, 1 or 2; most preferably 0 or 1, amino acid insertions, deletions, substitutions or a combination thereof with respect to the amino acid sequence of the VH of the indicated MFs.

It is shown in the Examples of WO2018/056821 A1, binding of the CD137-specific VH of MF6797 (SEQ ID NO: 49), is associated with the presence of amino acids comprising Arg66, Gly70 and Phe72 of the CD137 amino acid sequence.

In certain embodiments, the present disclosure therefore also provides the use or method of treatment of an isolated, synthetic or recombinant antibody, or a functional part, derivative and/or analogue thereof, that is able to bind to CD137, wherein the binding of said antibody or functional part, derivative or analogue to CD137 is associated with the presence of amino acids comprising Arg66, Gly70 and Phe72 of the CD137 amino acid sequence (SEQ ID NO: 117). In certain embodiments, the binding of said antibody or functional part, derivative or analogue to CD137 is also associated with an amino acid comprising Val71 of the CD137 amino acid sequence.

The term “Arg66” refers to the arginine residue at position 66 of the CD137 sequence. The term “Gly70” refers to the glycine residue at position 70 of the CD137 sequence according to SEQ ID NO 117. The term “Val71” refers to the valine residue at position 71 of the CD137 sequence. The term “Phe72” refers to the phenylalanine residue at position 72 of the CD137 sequence.

In certain embodiments, the multispecific antibody of the present disclosure comprises binding sites comprising a common light chain according to SEQ ID NO: 109 with 0-5 amino acid insertions, deletions, substitutions, additions or a combination thereof. In certain embodiments, each of the binding sites comprises a common light chain according to SEQ ID NO: 109 with 0-5 amino acid insertions, deletions, substitutions, additions or a combination thereof.

In certain embodiments, multispecific antibodies as described herein comprise a common light chain variable domain, preferably a common light chain variable region according to SEQ ID NO: 110 with 0-5 amino acid insertions, deletions, substitutions, additions or a combination thereof, preferably with 0, 1, 2, 3, or 4; more preferably with 0, 1, 2, or 3, more preferably with 0, 1 or 2; most preferably with 0 or 1, amino acid insertions, deletions, substitutions or a combination thereof. In certain embodiments, multispecific antibodies as described herein comprise a common light chain constant domain, preferably a common light chain constant region according to SEQ ID NO: 111 with 0-5 amino acid insertions, deletions, substitutions, additions or a combination thereof. The term ‘common light chain’ according to the present disclosure refers to light chains which may be identical or have some amino acid sequence differences while the binding specificity of the full length antibody is not affected. It is for instance possible within the scope of the definition of common light chains as used herein, to prepare or find light chains that are not identical but still functionally equivalent, e.g., by introducing and testing conservative amino acid changes, changes of amino acids in regions that do not or only partly contribute to binding specificity when paired with the heavy chain, and the like. The terms ‘common light chain’, ‘common LC’, ‘cLC’, ‘single light chain’ with or without the addition of the term ‘rearranged’ are all used herein interchangeably. The terms ‘common light chain variable region’, ‘common VL’, ‘common LCv’, ‘cLCv’, ‘single VL’ with or without the addition of the term ‘rearranged’ are all used herein interchangeably. In certain embodiments of the present disclosure, the multispecific antibody has a common light chain (variable region) that can combine with at least two, and preferably a plurality of heavy chains (variable regions) of different binding specificity to form antibodies with functional antigen binding domains (WO2004/009618, WO2009/157771). In certain embodiments, the common light chain (variable region) is a human light chain (variable region). In certain embodiments, a common light chain (variable region) has a germline sequence. In certain embodiments, the germline sequence is a light chain variable region that is frequently used in the human repertoire and has good thermodynamic stability, yield and solubility. In certain embodiments, the common light chain is the rearranged germline human kappa light chain IgVκ1-39*01/IGJκ1*01 (SEQ ID NO 109). In certain embodiments, the common light chain variable region is the variable region of the rearranged germline human kappa light chain IgVκ1-39*01/IGJκ1*01. In certain embodiments, the common light chain comprises a light chain variable region as presented in SEQ ID NO 110 with 0-5 amino acid insertions, deletions, substitutions, additions or a combination thereof, preferably with 0, 1, 2, 3, or 4; more preferably with 0, 1, 2, or 3, more preferably with 0, 1 or 2; most preferably with 0 or 1, amino acid insertions, deletions, substitutions or a combination thereof. In certain embodiments, the common light further comprises a light chain constant region, preferably a kappa light chain constant region. A nucleic acid that encodes the common light chain can be codon optimized for the cell system used to express the common light chain protein. The encoding nucleic acid can deviate from a germ-line nucleic acid sequence.

In certain embodiments, the multispecific antibody according to the use or method of the present disclosure is a full length antibody or a fragment of an antibody, for example a Fab fragment or a single-chain variable fragment (scFv). In certain embodiments, the multispecific antibody according to the use or method of the present disclosure is a full length antibody.

In certain embodiments, the multispecific antibody according to the use or method of the present disclosure is an IgG. In certain embodiments, the multispecific antibody is an IgG1 molecule without Fe effector function.

An Fc region mediates effector functions of an antibody, such as complement-dependent cytotoxicity (CDC), antibody-dependent cellular cytotoxicity (ADCC) and antibody-dependent cell phagocytosis (ADCP). Depending on the therapeutic antibody or Fc fusion protein application, it may be desired to either reduce or increase the effector function. Reduced effector functions are preferred in the present disclosure. Reduced effector function can be desired when an immune response is to be activated, enhanced or stimulated as in some of the embodiments of the present disclosure. Antibodies with reduced effector functions can be used to target cell-surface molecules of immune cells, among others. In certain embodiments, the CH2 region of the multispecific antibody of the present disclosure is engineered to reduce ADCC and/or CDC activity of the antibody. In certain embodiments, the CH3 region of the multispecific antibody is engineered to facilitate heterodimerization of the heavy chains.

Antibodies with reduced effector functions are preferably IgG antibodies comprising a modified CH2/lower hinge region, for instance to reduce Fc-receptor interaction or to reduce C1q binding. In certain embodiments the antibody of the present disclosure is an IgG antibody with a mutant CH2 and/or lower hinge domain such that interaction of the multispecific IgG antibody to an Fc-gamma receptor is reduced. In certain embodiments, CH2 region of the present disclosure comprises an amino acid sequence according to SEQ ID NO: 114 with 0-5 amino acid insertions, deletions, substitutions, additions or a combination thereof. In certain embodiments, a hinge region of the present disclosure comprises an amino acid sequence according to SEQ ID NO: 113 with 0-5 amino acid insertions, deletions, substitutions, additions or a combination thereof.

In certain embodiments, the CH3 region of the multispecific antibody is engineered to facilitate heterodimerization of the heavy chains. In certain embodiments, these variations are present to produce essentially only multispecific full length IgG molecules with amino acid substitutions at positions 351 and 366, e.g. L351K and T366K (numbering according to EU numbering) in the first CH3 domain (the ‘KK-variant’ heavy chain) and amino acid substitutions at positions 351 and 368, e.g. L351D and L368E in the second CH3 domain (the ‘DE-variant’ heavy chain), or vice versa. Homodimerization of DE-variant heavy chains (DE-DE homodimers) or KK-variant heavy chains (KK-KK homodimers) hardly occurs due to strong repulsion between the charged residues in the CH3-CH3 interface between identical heavy chains. In certain embodiments, the multispecific antibody of the present disclosure comprises CH3 domains according to SEQ ID NOs 115 and 116 with 0-5 amino acid insertions, deletions, substitutions, additions or a combination thereof, with the proviso that the DE/KK variants are not changed.

In certain embodiments, where the cancer is selected from any solid tumor harboring MSI-high alterations; endometrial cancer, in particular MSI-high endometrial cancer; lung cancer, in particular non-small cell lung cancer (NSCLC), more in particular NSCLC with high expression of PD-L1; and breast cancer, in particular metastatic breast cancer and triple negative breast cancer (TNBC), the multispecific antibody of the present disclosure is administered in a dose of between 10-1200 mg, preferably between 10-600 mg, 25-600 mg, 10-300 mg, or 25-300 mg, more preferably between 25-150 mg or 25-100 mg, most preferably between 25-75 mg. In certain embodiments, where the cancer is selected from any solid tumor harboring MSI-high alterations; cervical cancer, such as PD-L1 positive cervical cancer or cervical cancer with high expression of PD-L1; endometrial cancer, such as MSI-high endometrial cancer; lung cancer, such as non-small cell lung cancer (NSCLC), or PD-L1 positive NSCLC, or NSCLC with high expression of PD-L1; brain cancer, such as glioblastoma; and breast cancer, such as metastatic breast cancer or triple negative breast cancer (TNBC), the multispecific antibody of the present disclosure is administered in a dose of between 10-1200 mg, or between 10-600 mg, or between 25-600 mg, or between 10-300 mg, or between 25-300 mg, or between 10-75 mg, or between 10-50 mg, or between 25-150 mg, or between 25-100 mg, or between 25-75 mg, or between 25-50 mg. In certain embodiments where the cancer is selected from any solid tumor harboring MSI-high alterations; endometrial cancer, in particular MSI-high endometrial cancer; lung cancer, in particular non-small cell lung cancer (NSCLC), more in particular NSCLC with high expression of PD-L1; and breast cancer, in particular metastatic breast cancer and triple negative breast cancer (TNBC), the multispecific antibody is administered in a dose of between 50-100 mg or 75-125 mg. In certain embodiments where the cancer is selected from any solid tumor harboring MSI-high alterations; cervical cancer, such as PD-L1 positive cervical cancer or cervical cancer with high expression of PD-L1; endometrial cancer, such as MSI-high endometrial cancer; lung cancer, such as non-small cell lung cancer (NSCLC), or PD-L1 positive NSCLC, or NSCLC with high expression of PD-L1; brain cancer, such as glioblastoma; and breast cancer, such as metastatic breast cancer or triple negative breast cancer (TNBC), the multispecific antibody is administered in a dose of between 25-50 mg, 25-75 mg, 50-100 mg or 75-125 mg. In certain embodiments, where the cancer is selected from any solid tumor harboring MSI-high alterations; endometrial cancer, in particular MSI-high endometrial cancer; lung cancer, in particular non-small cell lung cancer (NSCLC), more in particular NSCLC with high expression of PD-L1; and breast cancer, in particular metastatic breast cancer and triple negative breast cancer (TNBC), the multispecific antibody is administered in a dose of 25 mg, 50 mg, 75 mg, or 100 mg. In certain embodiments, where the cancer is selected from any solid tumor harboring MSI-high alterations; cervical cancer, such as PD-L1 positive cervical cancer or cervical cancer with high expression of PD-L1; endometrial cancer, such as MSI-high endometrial cancer; lung cancer, such as non-small cell lung cancer (NSCLC), or PD-L1 positive NSCLC, or NSCLC with high expression of PD-L1; brain cancer, such as glioblastoma; and breast cancer, such as metastatic breast cancer or triple negative breast cancer (TNBC), the multispecific antibody is administered in a dose of 10 mg, 15 mg, 20 mg, 25 mg, 30 mg, 35 mg, 40 mg, 45 mg, 50 mg, 75 mg, or 100 mg.

In certain embodiments, where the cancer is selected from any solid tumor harboring MSI-high alterations; endometrial cancer, in particular MSI-high endometrial cancer; lung cancer, in particular non-small cell lung cancer (NSCLC), more in particular NSCLC with high expression of PD-L1; and breast cancer, in particular metastatic breast cancer and triple negative breast cancer (TNBC), the multispecific antibody according to the use or method of the present disclosure is administered in a flat dose of between 10-1200 mg, preferably between 10-600 mg, 25-600 mg, 10-300 mg, or 25-300 mg, more preferably between 25-150 mg or 25-100 mg, most preferably between 50-100 mg. In certain embodiments, where the cancer is selected from any solid tumor harboring MSI-high alterations; cervical cancer, such as PD-L1 positive cervical cancer or cervical cancer with high expression of PD-L1; endometrial cancer, such as MSI-high endometrial cancer; lung cancer, such as non-small cell lung cancer (NSCLC), or PD-L1 positive NSCLC, or NSCLC with high expression of PD-L1; brain cancer, such as glioblastoma; and breast cancer, such as metastatic breast cancer or triple negative breast cancer (TNBC), the multispecific antibody according to the use or method of the present disclosure is administered in a flat dose of between 10-1200 mg, or between 10-600 mg, or between 25-600 mg, or between 10-300 mg, or between 25-300 mg, or between 25-150 mg, or between 25-100 mg, or between 25-75 mg, or between 25-50 mg. In certain embodiments, where the cancer is selected from any solid tumor harboring MSI-high alterations; endometrial cancer, in particular MSI-high endometrial cancer; lung cancer, in particular non-small cell lung cancer (NSCLC), more in particular NSCLC with high expression of PD-L1; and breast cancer, in particular metastatic breast cancer and triple negative breast cancer (TNBC), the multispecific antibody is administered in a dose of 50 mg, 75 mg, or 100 mg. In certain embodiments, where the cancer is selected from any solid tumor harboring MSI-high alterations; cervical cancer, such as PD-L1 positive cervical cancer or cervical cancer with high expression of PD-L1; endometrial cancer, such as MSI-high endometrial cancer; lung cancer, such as non-small cell lung cancer (NSCLC), or PD-L1 positive NSCLC, or NSCLC with high expression of PD-L1; brain cancer, such as glioblastoma; and breast cancer, such as metastatic breast cancer or triple negative breast cancer (TNBC), the multispecific antibody is administered in a dose of 10 mg, 15 mg, 20 mg, 25 mg, 30 mg, 35 mg, 40 mg, 45 mg, 50 mg, 75 mg, or 100 mg.

In certain embodiments, the multispecific antibody according to the use or method of the present disclosure is administered in a flat dose.

In certain embodiments, the multispecific antibody according to the use or method of the present disclosure is administered in a dose, or flat dose, of between 50-150 mg, preferably between 75-150 mg, more preferably between 100-150 mg. In certain embodiments, the multispecific antibody according to the use or method of the present disclosure is administered in a dose, or flat dose, of between 50-150 mg, or between 75-150 mg, or between 100-150 mg.

In certain embodiments, the multispecific antibody according to the use or method of the present disclosure is administered in a dose, or flat dose, of between 75-125 mg.

In certain embodiments, the multispecific antibody according to the use or method of the present disclosure is administered in a dose, or flat dose, of between 50-100 mg, preferably between 75-100 mg. In certain embodiments, the multispecific antibody according to the use or method of the present disclosure is administered in a dose, or flat dose, of between 50-100 mg, or between 75-100 mg.

In certain embodiments, the multispecific antibody according to the use or method of the present disclosure is administered in a dose, or flat dose, of between 50-75 mg.

In certain embodiments, the multispecific antibody according to the use or method of the present disclosure is administered in a dose, or flat dose, of between 10-50 mg, preferably between 25-50 mg. In certain embodiments, the multispecific antibody according to the use or method of the present disclosure is administered in a dose, or flat dose, of between 10-50 mg, or between 25-50 mg.

In certain embodiments, the multispecific antibody according to the use or method of the present disclosure is administered once every week, once every two weeks or once every three weeks. In certain embodiments, the multispecific antibody according to the use or method of the present disclosure is administered once every two weeks. In certain embodiments, if the multispecific antibody according to the use or method of the present disclosure is administered once every week, the multispecific antibody is administered in a dose of between 10-100 mg, preferably between 15-75 mg. In certain embodiments, if the multispecific antibody according to the use or method of the present disclosure is administered once every week, the multispecific antibody is administered in a dose of between 10-100 mg, or between 15-75 mg, such as between 15-50 mg, or between 15-40 mg, or between 15-30 mg, or between 15-25 mg.

In certain embodiments, the multispecific antibody according to the use or method of the present disclosure is administered in a flat dose once every two weeks.

In certain embodiments, the multispecific antibody according to the use or method of the present disclosure is administered in a dose, or flat dose of 25, 30, 35, 40, 45, or 50 mg once every two weeks. In certain embodiments, the multispecific antibody according to the use or method of the present disclosure is administered in a dose, or flat dose of 25 mg once every two weeks. In certain embodiments, the multispecific antibody according to the use or method of the present disclosure is administered in a dose, or flat dose of 50 mg once every two weeks.

In certain embodiments, the multispecific antibody according to the use or method of the present disclosure is administered as a single agent therapy.

In certain embodiments, the multispecific antibody according to the use or method of the present disclosure is administered intravenously.

In certain embodiments, the multispecific antibody according to the use or method of the present disclosure is administered over a period of between 30 minutes to 4 hours, preferably between 1 to 3 hours, most preferably 2 hours.

In certain embodiments, the multispecific antibody according to the use or method of the present disclosure is administered over a period of between 30 minutes to 4 hours, preferably between 1 to 3 hours, most preferably 2 hours.

In certain embodiments, the multispecific antibody according to the use or method of the present disclosure is administered intravenously as a flat dose over a period of 2 hours every 14 days in 28-day cycles.

In certain embodiments, the multispecific antibody according to the use or method of the present disclosure is administered intravenously as a flat dose of 25 mg over a period of 2 hours every 14 days in 28-day cycles. In certain embodiments, the multispecific antibody according to the use or method of the present disclosure is administered intravenously as a flat dose of 30 mg over a period of 2 hours every 14 days in 28-day cycles. In certain embodiments, the multispecific antibody according to the use or method of the present disclosure is administered intravenously as a flat dose of 40 mg over a period of 2 hours every 14 days in 28-day cycles. In certain embodiments, the multispecific antibody according to the use or method of the present disclosure is administered intravenously as a flat dose of 50 mg over a period of 2 hours every 14 days in 28-day cycles. In certain embodiments, the multispecific antibody according to the use or method of the present disclosure is administered intravenously as a flat dose of 60 mg over a period of 2 hours every 14 days in 28-day cycles. In certain embodiments, the multispecific antibody according to the use or method of the present disclosure is administered intravenously as a flat dose of 70 mg over a period of 2 hours every 14 days in 28-day cycles. In certain embodiments, the multispecific antibody according to the use or method of the present disclosure is administered intravenously as a flat dose of 75 mg over a period of 2 hours every 14 days in 28-day cycles.

In certain embodiments, the multispecific antibody according to the use or method of the present disclosure is formulated as a liquid in a concentration of 1 mg/mL to 100 mg/mL, preferably at or around 20 mg/mL. In certain embodiments, the multispecific antibody according to the use or method of the present disclosure is formulated as a liquid in a concentration of 1 mg/mL to 100 mg/mL, or at or around 20 mg/mL.

In certain embodiments, the multispecific antibody according to the use or method of the present disclosure is administered prior to, simultaneously, or more preferably after, an antihistamine, a nonsteroidal anti-inflammatory drug (NSAID), a narcotic, an intravenous fluid, an antipyretic, a bronchodilator, oxygen, a corticosteroid (IV/oral), a vasopressor, or any combination thereof, is administered to reduce infusion-related reactions.

Premedication with the indicated substances may be opted for subjects in case an infusion-related reaction is experienced to prevent and mitigate the incidence and severity thereof.

In certain embodiments, the multispecific antibody according to the use or method of the present disclosure is administered after the subject has been pretreated with standard of care therapy, such as chemotherapy, immunotherapy or targeted therapy, for advanced metastatic disease.

In certain embodiments, the multispecific antibody according to the use or method of the present disclosure is administered to a subject that has not been treated with an anti-PD-L1 agent, such as an anti-PD-L1 antibody, or T-cell agonist.

In certain embodiments, the cancer is selected from; endometrial cancer, in particular MSI-high endometrial cancer; lung cancer, in particular non-small cell lung cancer (NSCLC), more in particular NSCLC with high expression of PD-L1; and breast cancer, in particular metastatic breast cancer and triple negative breast cancer (TNBC). In certain embodiments, the cancer is selected from: solid tumors harboring MSI-high alterations; cervical cancer, such as PD-L1 positive cervical cancer or cervical cancer with high expression of PD-L1; endometrial cancer, such as MSI-high endometrial cancer; lung cancer, such as non-small cell lung cancer (NSCLC), or PD-L1 positive NSCLC, or NSCLC with high expression of PD-L1; brain cancer, such as glioblastoma; and breast cancer, such as metastatic breast cancer or triple negative breast cancer (TNBC).

Solid tumors harboring MSI-high alterations include for instance, but are not limited to, MSH-high colorectal cancer, MSI-high gastric cancer, MSI-high breast cancer, MSI-high prostate cancer, MSI-high bladder cancer, MSI-high ovarian cancer, MSI-high endometrial cancer, MSI-high endometrial carcinoma, pancreatic ductal adenocarcinoma, follicular thyroid cancer, and adrenocortical cancer.

PD-L1 expression is determined by clinicians of ordinary skill in the art, see exemplary methods in de Ruiter et al. (2021), incorporated herewith in its entirety. High expression as determined by any one of these methods shall constitute high expression for purposes of this disclosure. When evaluated by tumor proportion score (TPS), a TPS score of greater than or equal to 1% is a PD-L1 positive cancer. When evaluated by combined positive score (CPS), a score of greater than or equal to 1% is a PD-L1 positive cancer. A cancer is PD-L1 high if having a PD-L1 expression score of 10% or higher, 20% or higher, 30% or higher, 40% or higher, or 50% or higher when determined using the tumor proportion score (TPS). A cancer is PD-L1 high if having a PD-L1 expression score of 5% or higher, 15% or higher, or 20% or higher, when determined using the combined positive score (CPS). If evaluated by multiple such methods, if any one or more method identifies a tumor as PD-L1 positive or high, it meets the criteria for the present disclosure.

In certain embodiments, the subject has not received prior treatment with an immune checkpoint inhibitor. In certain embodiments, the subject has not received prior treatment with an immune checkpoint inhibitor and is being treated for an MSI-high cancer. In certain embodiments, the subject has not received prior treatment with an immune checkpoint inhibitor and is being treated for endometrial cancer, in particular MSI-high endometrial cancer. In certain embodiments, the subject has not received prior treatment with an immune checkpoint inhibitor and is being treated for lung cancer, in particular non-small cell lung cancer. In certain embodiments, the subject has not received prior treatment with an immune checkpoint inhibitor and is being treated for breast cancer, in particular metastatic breast cancer and triple negative breast cancer (TNBC).

In certain embodiments, the multispecific antibody according to the use or method of the present disclosure increases the number of CD8⁺ T cells, in particular in breast cancer, more in particular in immunodeficient mice bearing human MDA-MB-231 tumors.

In certain embodiments, the multispecific antibody according to the use or method of the present disclosure is significantly less toxic than a combination of urelumab and atezolizumab, preferably as determined in the same study, in particular in breast cancer, more in particular in immunodeficient mice bearing human MDA-MB-231 tumors.

In certain embodiments, the multispecific antibody according to the use or method of the present disclosure does not induce graft-versus-host disease.

The term “antibody” as used herein means a proteinaceous molecule, preferably belonging to the immunoglobulin class of proteins, containing one or more variable domains that bind an epitope on an antigen, where such domains are derived from or share sequence homology with the variable domain of an antibody. Antibodies for therapeutic use are preferably as close to natural antibodies of the subject to be treated as possible (for instance human antibodies for human subjects). Antibody binding can be expressed in terms of specificity and affinity. The specificity determines which antigen or epitope thereof is specifically bound by the binding domain. The affinity is a measure for the strength of binding to a particular antigen or epitope. Antibodies such as the multispecific antibodies of the present disclosure typically comprise the constant domains (Fc part) of a natural antibody, which may be engineered as described elsewhere herein, for instance to reduce ADCC and/or CDC activity.

A “multispecific antibody” refers to an antibody comprising at least two binding sites with different antigen or epitope specificity. In certain embodiments, one or more of the antigen binding sites comprises an immunoglobulin VH/VL pair. In certain embodiments, each of the antigen binding sites comprises an immunoglobulin VH/VL pair.

In certain embodiments, a multispecific antibody according to the present disclosure has no more than two antigen binding sites. This means that the antigen binding part of such multispecific antibody consists of two antigen binding sites, without the presence of additional antigen binding sites. In certain embodiments, each of the two antigen binding sites contains an immunoglobulin VH/VL pair.

In certain embodiments, the VL in each VH/VL pair is similar. In certain embodiments, the VL in each VH/VL pair is identical. In certain embodiments, the multispecific antibody is a full length antibody which has one heavy/light (H/L) chain combination that binds an extracellular part of CD137 and one H/L chain combination that binds an extracellular part of a member of the B7 family. In certain embodiments, the light chain in said first H/L chain combination is similar to the light chain in said second H/L chain combination. In certain embodiments, the light chains in the first and second H/L chain combinations are identical.

In certain embodiments, the multispecific antibody is a bispecific antibody.

The term ‘bispecific antibody’ means that one part of the antibody binds to one epitope on an antigen whereas a second part binds to a different epitope on either the same antigen, or a different antigen. The different epitopes are typically present on different antigens. The different epitopes can, however, also be present on the same antigen. Dependent on the expression level, (sub-)cellular localization and stoichiometry of the two antigens recognized by a bispecific antibody, both Fab arms of the antibody may or may not simultaneously bind their epitope. One arm of the bispecific antibody typically contains the variable domain of one antibody and the other arm contains the variable domain of another antibody (i.e. one arm of the bispecific antibody is formed by one heavy chain paired with one light chain whereas the other arm is formed by a different heavy chain paired with a light chain). In certain embodiments, the heavy chain variable regions of a bispecific antibody of the present disclosure are different from each other, whereas the light chain variable regions are the same in the bispecific antibodies of the present disclosure. A bispecific antibody wherein the different heavy chain variable regions are associated with the same or a common, light chain variable region is also referred to as a bispecific antibody with a common light chain variable region (cLcv). In certain embodiments, the light chain constant region is also the same. Such bispecific antibodies are referred to as having a common light chain (cLc).

Certain preferred embodiments are immunoglobulins having an IgG format, providing the advantage that the half-lives of bivalent binding molecules/antibodies/variants according to the invention are typically longer as compared to multivalent compounds. Moreover, the immunogenicity of bivalent binding molecules according to the invention is typically lower as compared to multivalent compounds. Molecules/antibodies/variants according to these embodiments preferably maintain the structure of natural IgGs and therefore maintain all benefits associated to that structure of natural IgGs.

A “variant” of an antibody or multispecific antibody as described herein comprises a functional part, derivative and/or analogue of the antibody or multispecific antibody. The variant may be a fragment of an antibody for example a Fab fragment. The variant may be a single-chain variable fragment (scFv). The variant maintains the binding specificity of the antibody. The functional part, derivative and/or analogue maintains the binding specificity of the antibody. Binding specificity is defined by capacity to bind an extracellular part of a first membrane protein and a second membrane protein as described herein. A variant may have amino acid insertions, deletions, substitutions, or a combination thereof relative to a given amino acid sequence (e.g. SEQ ID NO.), at most 15, preferably 0, 1, 2, 3, 4, 5, 6, 7, 8, 9 or 10, more preferably 0, 1, 2, 3, 4 or 5, more preferably 0, 1, 2, 3, or 4; more preferably 0, 1, 2, or 3, more preferably 0, 1 or 2; most preferably 0 or 1 amino acid insertions, deletions, substitutions or a combination thereof with respect to the amino acid sequence of a given SEQ ID NO.

As used herein, the term “antigen binding site” means a site of a binding molecule or antibody that specifically binds an epitope of an antigen. Such antigen binding site is preferably derived from or shares sequence homology with the variable domain of an antibody, in particular the CDR regions thereof. In some preferred embodiments, said antigen binding site is an immunoglobulin variable domain, formed by an immunoglobulin VH/VL pair. In other embodiments, said antigen binding site is derived from an antibody mimetic, such as for instance from an affibody molecule, affilin, affimer, affitin, alphabody, anticalin, avimer, DARPin, fynomer, kunitz domain peptide or monobody, which are described herein before.

The term ‘full length’ according to the invention is defined as comprising an essentially complete antibody, without one or more artificially added moieties with a size of larger than 20 amino acid residues, such as for instance additional antigen binding sites or additional activation sites or additional ligands or additional ligand binding moieties. A full length antibody however does not necessarily have all functions of an intact antibody. For the avoidance of doubt, a full length antibody contains two heavy and two light chains. Each chain contains constant (C) and variable (V) regions, which can be broken down into domains designated CH1, CH2, CH3, VH for the heavy chain, and CL, VL for the light chain. The domains of the heavy chains are preferably present in the order of a natural antibody (VHCH1-CH2-CH3; meaning that the VH domain is adjacent to the CH1 domain, followed by a CH2 domain and subsequently followed by a CH3 domain). The domains of the light chains are also preferably present in the order of a natural antibody (VL-CL; meaning that the VL domain is adjacent to the CL domain). An antibody binds to antigen via the variable domains contained in the Fab fragment portion. The antibody can interact with molecules and cells of the immune system through the constant domains, mostly through the Fc portion.

In certain embodiments, full length IgG antibodies are preferred because of their typically favorable half-life and the desire to stay as close to fully autologous (human) molecules for reasons of immunogenicity. In certain embodiments, a multispecific antibody of the present disclosure is a full length IgG1, a full length IgG2, a full length IgG3 or a full length IgG4 antibody.

Full length antibodies encompass antibodies wherein mutations may be present that provide desired characteristics or are just alternatives to the ones in the original chain. Such mutations typically are not deletions of substantial portions of any of the regions. However, antibodies wherein one or several amino acid residues are inserted, deleted, substituted or a combination thereof, without essentially altering the antigen binding characteristics of the resulting antibody are embraced within the term “full length antibody”. For instance, an IgG antibody can have 1-20 amino acid residue insertions, substitutions, deletions or a combination thereof in the constant region.

### EXAMPLES

### Example 1: Multispecific Antibodies Binding PD-L1 and CD137

Multispecific antibodies comprising heavy chain variable regions as mentioned in Table 1 were obtained as described in WO2018/056821.

TABLE 1
SEQ ID NOs for heavy chain variable regions of multispecific
antibodies binding CD137 and PD-L1.

| CD137 binding | | PD-L1 binding | |
|---|---|---|---|
| domain ID | SEQ ID NOs | domain ID | SEQ ID NOs |
| MF6783 | SEQ ID NO: 1 | MF5554 | SEQ ID NO: 53 |
| MF6861 | SEQ ID NO: 5 | MF5576 | SEQ ID NO: 57 |
| MF6795 | SEQ ID NO: 9 | MF5578 | SEQ ID NO: 59 |
| MF6808 | SEQ ID NO: 13 | MF9375 | SEQ ID NO: 62 |
| MF6798 | SEQ ID NO: 17 | MF9376 | SEQ ID NO: 64 |
| MF6754 | SEQ ID NO: 20 | MF7702 | SEQ ID NO: 67 |
| MF6763 | SEQ ID NO: 24 | MF5359 | SEQ ID NO: 69 |
| MF6744 | SEQ ID NO: 28 | MF5377 | SEQ ID NO: 73 |
| MF6785 | SEQ ID NO: 31 | MF5382 | SEQ ID NO: 77 |
| MF6825 | SEQ ID NO: 35 | MF5424 | SEQ ID NO: 81 |
| MF6737 | SEQ ID NO: 39 | MF5426 | SEQ ID NO: 85 |
| MF6749 | SEQ ID NO: 43 | MF5439 | SEQ ID NO: 89 |
| MF6788 | SEQ ID NO: 46 | MF5442 | SEQ ID NO: 92 |
| MF6797 | SEQ ID NO: 49 | MF5553 | SEQ ID NO: 96 |
| | | MF5557 | SEQ ID NO: 97 |
| | | MF5561 | SEQ ID NO: 100 |
| | | MF5576 | SEQ ID NO: 103 |
| | | MF5594 | SEQ ID NO: 104 |
| | | MF5708 | SEQ ID NO: 107 |

TABLE 2
Any of the heavy chain variable regions binding CD137 can be combined with any
of the heavy chain variable regions binding PD-L1 in the multispecific antibodies
of the present disclosure. Variants of the variable heavy chain regions may also
be combined in the multispecific antibodies of the present disclosure.

| | MF6783 | MF6861 | MF6795 | MF6808 | MF6798 | MF6754 | MF6763 |
|---|---|---|---|---|---|---|---|
| MF5554 | X | X | X | X | X | X | X |
| MF5576 | X | X | X | X | X | X | X |
| MF5578 | X | X | X | X | X | X | X |
| MF9375 | X | X | X | X | X | X | X |
| MF9376 | X | X | X | X | X | X | X |
| MF7702 | X | X | X | X | X | X | X |
| MF5359 | X | X | X | X | X | X | X |
| MF5377 | X | X | X | X | X | X | X |
| MF5382 | X | X | X | X | X | X | X |
| MF5424 | X | X | X | X | X | X | X |
| MF5426 | X | X | X | X | X | X | X |
| MF5439 | X | X | X | X | X | X | X |
| MF5442 | X | X | X | X | X | X | X |
| MF5553 | X | X | X | X | X | X | X |
| MF5557 | X | X | X | X | X | X | X |
| MF5561 | X | X | X | X | X | X | X |
| MF5576 | X | X | X | X | X | X | X |
| MF5594 | X | X | X | X | X | X | X |
| MF5708 | X | X | X | X | X | X | X |
| | MF6744 | MF6785 | MF6825 | MF6737 | MF6749 | MF6788 | MF6797 |
| MF5554 | X | X | X | X | X | X | X |
| MF5576 | X | X | X | X | X | X | X |
| MF5578 | X | X | X | X | X | X | X |
| MF9375 | X | X | X | X | X | X | X |
| MF9376 | X | X | X | X | X | X | X |
| MF7702 | X | X | X | X | X | X | X |
| MF5359 | X | X | X | X | X | X | X |
| MF5377 | X | X | X | X | X | X | X |
| MF5382 | X | X | X | X | X | X | X |
| MF5424 | X | X | X | X | X | X | X |
| MF5426 | X | X | X | X | X | X | X |
| MF5439 | X | X | X | X | X | X | X |
| MF5442 | X | X | X | X | X | X | X |
| MF5553 | X | X | X | X | X | X | X |
| MF5557 | X | X | X | X | X | X | X |
| MF5561 | X | X | X | X | X | X | X |
| MF5576 | X | X | X | X | X | X | X |
| MF5594 | X | X | X | X | X | X | X |
| MF5708 | X | X | X | X | X | X | X |

### Example 2—Ex Vivo Activity on T Cell Function in Endometrial Tumor Explants

To investigate the activity of multispecific antibodies binding CD137 and PD-L1 under therapeutically relevant conditions, fresh tumor explants containing tumor specific effector T cells and regulatory T cells (Tregs) were evaluated.

Five surgically resected primary endometrial tumors were dissociated into single cell suspensions. Tumor samples were then analyzed by flow cytometry for cell counts or incubated with a multispecific antibody binding CD137 and PD-L1 or reference antibodies in the presence of soluble anti-CD3 antibody to measure IFNγ production. Any multispecific antibody binding CD137 and PD-L1 described herein can be used, including the one comprising the VH of MF6797 and MF7702, as exemplified herein. Reference antibodies included a monospecific urelumab analog, monospecific atezolizumab analog, and a combination of the urelumab and atezolizumab analogs. The atezolizumab analog was made based on the information disclosed in WO 2010/077634. The information used to make the urelumab analog was obtained from WO 2005/035584.

Tumor samples were stained with an 18-color flow cytometry phenotyping panel. Live cells were distinguished from dead cells using staining of dead cells (Biolegend, cat. no. 423110). T cells were identified by first isolating CD45⁺ cells (BD Biosciences, cat. no. 560178), then excluding CD14⁺ cells (Thermo Fisher Scientific, cat. no. 15-0149-42) and CD19⁺ cells (Biolegend, cat. no. 302210), and then selecting CD3⁺ cells (BD Biosciences, cat. no. 563546). T cells were then separated into CD4⁺ cells (BD Biosciences, cat. no. 564305) and CD8⁺ cells (BD Biosciences, cat. no. 564804). CD4⁺ cells were further separated into Treg cells by double positive FoxP3 staining (Thermo Fisher Scientific, cat. no. 25-4777-42) and CD25⁺ cells (BD Biosciences, cat. no. 563159). The CD4⁺Treg⁻, Treg⁺ and CD8⁺ T cell subsets were then classified by their positive expression of CD226 (Biolegend, cat. no. 338330), ICOS (Thermo Fisher Scientific, cat. no. 62-9948-42), CTLA-4 (BD Biosciences, cat. no. 555853), CD137 (BD Biosciences, cat. no. 745256), OX40 (Biolegend, cat. no. 350018), Lag-3 (Biolegend, cat. no. 369312), Tim-3 (BD Biosciences, cat. no. 565564), IL-10 (Biolegend, cat. no. 501411), GITR (BD Biosciences, cat. no. 747661), and PD-L1 (BD Biosciences, cat. no. 565188). Stained samples were run on a LSR Fortessa X-20 cell analyzer (BD Biosciences) and gates drawn using fluorescence minus one (FMO) controls.

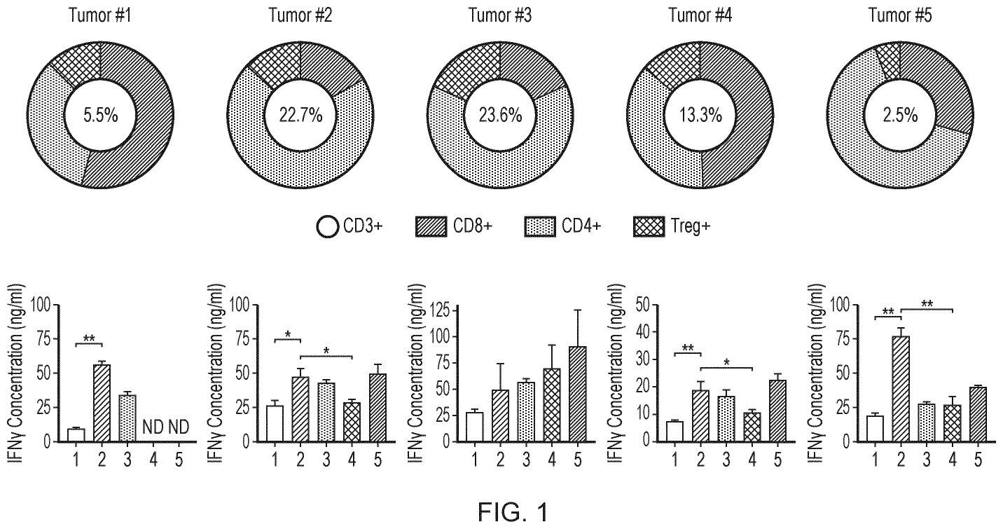

The percentage of CD3⁺ T cells, the proportion of CD4⁺, CD8⁺ and Treg subsets and the level of IFNγ production was heterogeneous between tumor samples (FIG. 1; upper panel). Treatment with the multispecific antibody increased IFNγ production in all tumor samples relative to the negative control antibody and, to a lesser extent, to comparator urelumab and atezolizumab analogs, and a combination thereof. This effect was observed even in the context of high Treg numbers (FIG. 1; lower panel).

### Example 3—In Vivo Efficacy in Immunodeficient Mice Bearing Human A549 Tumors

Efficacy evaluation of multispecific antibodies binding CD137 and PD-L1 In vivo was carried out in murine xenograft models. Any multispecific antibody binding CD137 and PD-L1 described herein can be used, including the one comprising the VH of MF6797 and MF7702, as exemplified herein. The binding arms of this multispecific antibody do not cross react with the murine homologues of PD-L1 and CD137. Therefore, a humanized murine xenograft model was used. In this model, human transgenic Ly95 T cells that resemble patient tumor-infiltrating lymphocytes (TILs) were adoptively transferred into immunodeficient mice bearing human A549 tumors. This permits the effects of antibody treatment on such TILs to be studied in a non-human model that mimics the human tumor microenvironment. The NSCLC cell line A549 expresses NY-ESO antigen in the appropriate HLA context and was modified for the study to stably express high levels of PD-L1. Ly95 cells express PD-1 and CD137.

NSG mice (6-8 weeks of age; The Jackson Laboratory, Bar Harbor, ME) were first inoculated subcutaneously with 5×10⁶ A549-A2-ESO tumor cells suspended in 100 μL serum-free culture medium and matrigel membrane matrix (Corning) in equal volumes. After tumors were established (mean volume of 150 mm³), the mice were randomized into six groups of seven mice whereby one group received a single intravenous tail-vein injection of PBS alone, and five groups were injected with PBS containing 10×10⁶ NY-ESO1-reactive Ly95 TCR construct-expressing human T cells. The five groups that had undergone adoptive transfer with the tumor-specific transgenic Ly95 T cells were subsequently treated intraperitoneally every five days with PBS, atezolizumab (5 mg/kg), urelumab analog (5 mg/kg), equimolar mix of atezolizumab and urelumab analog (5 mg/kg), or the multispecific antibody (5 mg/kg). Over a period of four weeks, tumor volume was recorded twice a week using a study log system.

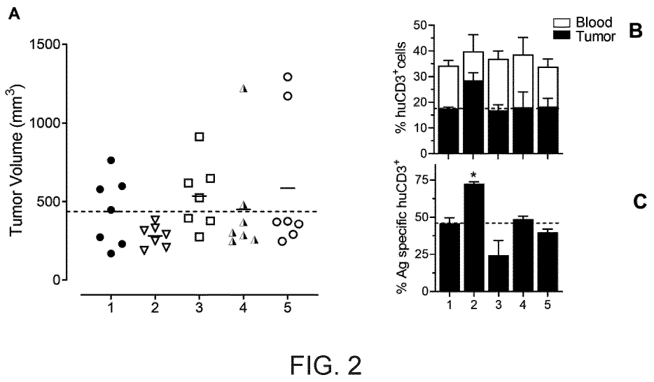

In mice engrafted with A549 PD-L1hi cells, treatment with urelumab analog, atezolizumab, or a combination of urelumab analog and atezolizumab, along with Ly95 cells, did not significantly alter tumor growth compared to control (FIG. 2A). All mice treated with the multispecific antibody were able to control tumor growth (FIG. 2A). Importantly, multispecific antibody dependent tumor growth inhibition was clearly associated with a skewed distribution of adoptively transferred huCD3⁺Ly95 cells to tumor vs blood (FIG. 2B) and increased NY-ESO antigen-specific T cells within the tumor compared to mice treated with controls (FIG. 2C).

### Example 4—In Vivo Efficacy in Immunodeficient Mice Bearing Human MDA-MB-231 Tumors

To evaluate the impact of treatment with multispecific antibodies binding CD137 and PD-L1 in the context of a heterogenous T cell population, NSG mice were engrafted with human CD34⁺ hematopoietic stem cells after which the PD-L1 expressing human MDA-MB-231 breast cancer line was implanted. Any multispecific antibody binding CD137 and PD-L1 described herein can be used, including the one comprising the VH of MF6797 and MF7702, as exemplified herein.

To generate human stem cell-engrafted NSG mice, immunodeficient NSG mice (6-8 weeks of age; The Jackson Laboratory, Bar Harbor, ME) received 15 mg/kg intraperitoneal busulfan (Busilvex, Pierre Fabre) and 24 hours later an intravenous injection containing 1×10⁵ human CD34⁺ cord blood cells (purchased from STEMCELL Technologies) as previously described (Ishikawa F. et al. Development of functional human blood and immune systems in NOD/SCID/IL2 receptor γ chain null mice. Blood. 2005 Jan. 9; 106(5):1565-73). The experiment only included mice that had >25% huCD45⁺ cells and T cell levels above 80 counts/μL in peripheral blood. Mice were inoculated subcutaneously with a total of 3×10⁶ MDA-MB-231 tumor cells suspended in 100 μL of serum-free culture medium and matrigel matrix (Corning) in equal volumes. When tumors reached approximately 80-100 mm³, mice were randomized into the following groups (n=7 per group): 1) Fc-silenced IgG1 control (5 mg/kg); 2) atezolizumab (5 mg/kg); 3) urelumab analog (5 mg/kg); 4) equimolar mix of atezolizumab and urelumab analog (5 mg/kg); 5) pembrolizumab (5 mg/kg); and 6) multispecific antibody binding CD137 and PD-L1 (0.5 and 5 mg/kg). Antibodies were diluted in PBS (Life Technologies) and administered intraperitoneally. Animals were dosed intraperitoneally once every 5 days for a period of 31 days. Tumors were measured using calipers, and tumor volume was calculated by assimilating them to an ellipsoid using the formula: l (length)×w² (width)×½. Statistical significance was determined by one-way ANOVA. Body weights were also monitored throughout the study.

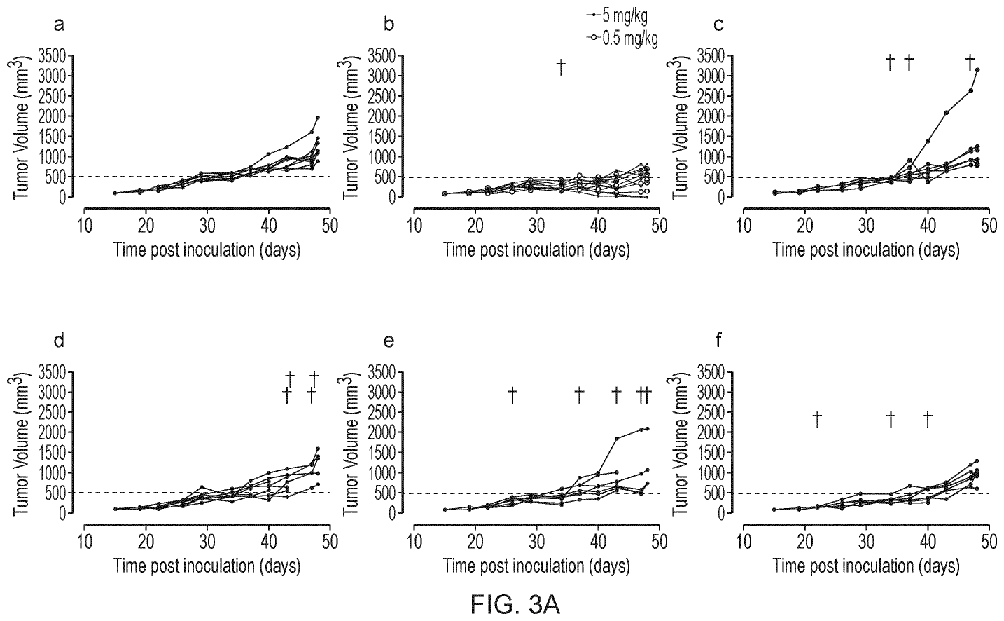

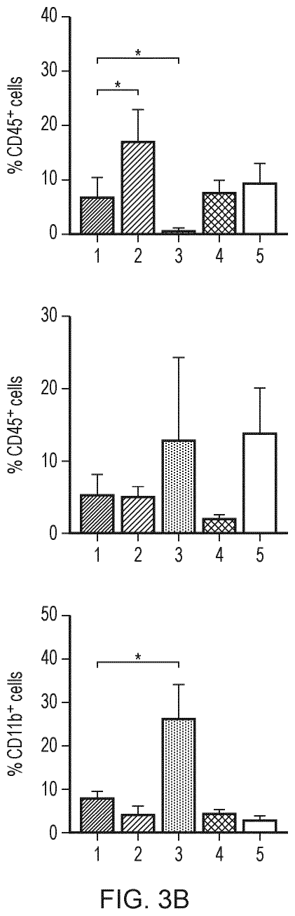

In animals receiving control IgG, tumors grew progressively over the first 40 days after which more rapid growth kinetics were observed in most animals (FIG. 3Aa). The group treated with CD137 agonist, urelumab analog showed a very similar tumor growth pattern to control (FIG. 3Ac). In the groups treated with pembrolizumab (FIG. 3f), atezolizumab (FIG. 3Ad), or the combination of atezolizumab and urelumab analog (FIG. 3Ae), there was evidence of tumor growth control in some mice but, by the end of the observation period, tumors in all surviving mice were growing rapidly. In contrast, all animals treated with the multispecific antibody experienced tumor control including two complete responses and showed slower growth kinetics at the end of the treatment period (FIG. 3Ab). A cohort of mice treated with a 10-fold lower dose of the multispecific antibody (0.5 mg/kg) also showed similar inhibition of tumor growth (FIG. 3Ab). Consistent with the NY-ESO model, analysis of TILs in this model showed that treatment with the multispecific antibody resulted in an increased frequency of CD8⁺ T cells in the tumor (FIG. 3B; upper graph). Urelumab analog treatment reduced the number of CD8⁺ cells in the tumor and this was associated with an increase in the frequency of CD4⁺ T cells and PD-L1⁺ monocytes. These results demonstrate that the potent T cell agonism observed with the multispecific antibody in vitro also translated into PD-L1 dependent tumor control in vivo.

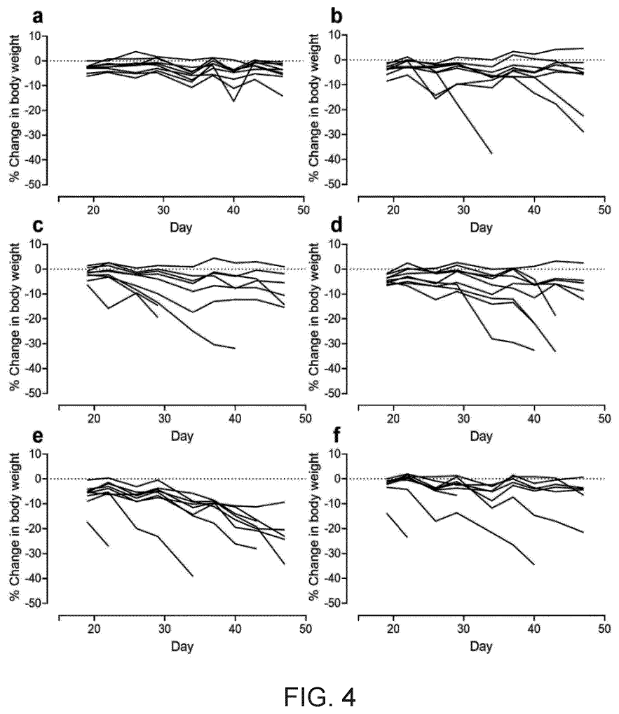

It was also noted that some mice in the pembrolizumab, atezolizumab or urelumab analog groups and all mice in the combination treatment group exhibited ruffled fur and skin and a hunched posture, in some cases associated with excessive weight loss (FIG. 4), that required euthanasia (3-5 mice in each group). No animals in the control or multispecific antibody groups showed signs of stress (although one animal in the multispecific antibody group was euthanized for unrelated reasons). In particular, it was observed that the combination treatment of urelumab analog and atezolizumab (FIG. 4e) was significantly more toxic than control or treatment with the multispecific antibody (FIG. 4b).

### Example 5—a Phase 1 Dose-Escalation Single Agent Study for Dose Finding, Safety, and Preliminary Efficacy of a Multispecific Antibody Targeting CD137 and PD-L1 in Participants with Advanced or Metastatic Malignancies

Treatment Groups and Duration:

An open-label, non-randomized, Phase 1 study was initiated to determine the safety, tolerability, and preliminary efficacy of a multispecific antibody targeting CD137 and PD-L1 in adult participants with advanced or metastatic malignancies.

The following multispecific antibodies are suitable for use in this study and for use in the methods of the invention: MF6797×MF7702, MF6763×MF5442, MF6754×MF5561, MF6785×MF5439 and MF6785×MF5442, preferably MF6797×MF7702. Each multispecific antibody comprises two VH's as specified by the MF numbers capable of binding CD137 and PD-L1 respectively, an Fc region with a KK/DE CH3 heterodimerization domain as indicated by SEQ ID NO:115 and SEQ ID NO:116, respectively, a CH2 domain as indicated by SEQ ID NO:114, a CH1 domain as indicated by SEQ ID NO: 112, and a common light chain as indicated by SEQ ID NO: 109.

A dose escalation study was performed to determine the MTD and/or RDE of a multispecific antibody in participants with advanced or recurrent/metastatic solid tumors. Participants received escalating doses of one of the exemplary multispecific antibodies indicated above, further referred to herein as the “study antibody” every 2 weeks until MTD or RDE was reached. The duration of each treatment cycle was 28 days. A total of 10 dosages were planned: 0.4 mg, 1.2 mg, 3.5 mg, 10 mg, 25 mg, 75 mg, 150 mg, 300 mg, 600 mg, and 1200 mg.

Overall Study Design

This is an open-label, Phase 1 dose escalation study to determine the safety, tolerability, and preliminary efficacy of the study antibody in adult participants with advanced or metastatic malignancies that will be conducted in two parts. The study antibody will be administered intravenously as a flat dose over 2 hours every 14 days in 28-day cycles.

Part 1: Dose Escalation

Part 1 is a dose escalation to determine the MTD and/or RDE of the study antibody administered every 14 days in participants with advanced or metastatic solid tumors, regardless of PD-L1 expression. During dose escalation, cohorts of participants will be treated with the study antibody until the MTD is reached or a lower recommended dose(s) is established. The dose escalation will be guided by an adaptive BLRM following the escalation with overdose control principle.

During the course of the dose escalation, additional cohorts of up to 6 participants may be enrolled at any planned or intermediate dose level below the next dose level or the MTD in order to better characterize safety, PK, and/or pharmacodynamic activity. A maximum of 5 participants with a given tumor type may be enrolled across all dose levels in Part 1, unless the medical monitor approves additional enrollment in that tumor type.

Part 1 enrollment will commence at Dose Level 1 (the study antibody at 0.4 mg IV) with the option to escalate or de-escalate based on defined criteria. Dose-limiting toxicities occurring up to and including Day 28 will guide dose escalation/de-escalation and determination of the RDE or MTD. However, late-onset immune-mediated toxicities through 90 days after the start of study treatment will be considered when assessing safety, and, therefore, a lower RDE or MTD may subsequently be determined based on relevant toxicities that become evident after Day 28. If the lowest dose level is not deemed safe, then Part 1 enrollment will be halted.

Part 2: Dose Confirmation/Safety Expansion

Part 2 is a dose expansion to confirm the dose of the study antibody through further evaluation of safety, tolerability, PK, preliminary antitumor activity, and functional target engagement.

Participants with advanced or metastatic tumors, regardless of PD-L1 expression, will be enrolled as follows:
Participants with anti-PD-1 therapy relapsed or refractory NSCLC;
Participants with anti-PD-1 therapy refractory MSI-H/dMMR tumors;
Participants with immunotherapy-naive TNBC;
Participants with tumor histology from Part 1 for which a preliminary efficacy was observed with the study antibody.

Up to two confirmatory dose levels may be explored. Confirmatory dose levels will be selected based on PK, antitumor, and pharmacodynamic activity including receptor modulation, safety, and tolerability.

The initial enrollment will be limited to 10 or 20 participants per indication per confirmatory dose level. If at least 1 participant has a confirmed response in 1 or both confirmatory dose levels, the indication may expand to a total of 40 participants across dose levels. If three or more indications enrolled in Part 2 show clinical activity (i.e., >1 confirmed response out of the first 10 participants), each indication may be allowed to expand up to 40 participants. The decision for expansion of each indication will be determined by the Study Steering Committee.

If there is insufficient evidence of clinical activity in Part 1 as determined by the Study Steering Committee, only 1 dose will be evaluated in Part 2. This cohort will contain a minimum of 20 participants with PD-L1 positive tumors as assessed by Ventana PD-L1 SP263 assay with a CPS cutoff of ≥1%. If 20 participants are found to have PD-L1 negative tumors prior to full enrollment of Part 2, subsequent participants with PD-L1 negative tumors will be ineligible for enrollment in Part 2.

Inclusion Criteria

Participants are eligible to be included in the study only if all of the following criteria apply.

Participants with NSCLC, MSI-H/dMMR tumors, or TNBC must meet all criteria listed here for “all participants” and the applicable Tumor-Specific Criteria listed below to confirm eligibility.

All Participants
Ability to comprehend and willingness to sign a written ICF for the study;
At least 18 years of age at the time of signing the informed consent;
Willingness and ability to conform to and comply with all Protocol requirements, including, all scheduled visits, and Protocol procedures;
Life expectancy of ≥12 weeks;
ECOG performance status of 0 or 1;
Part 1: Histologically or cytologically confirmed advanced metastatic solid tumors, that are considered non-amenable to surgery or other curative treatments or procedures (if applicable);
Part 2: Participants must have a diagnosis of one of the following tumor types and meet the applicable tumor-specific criteria: NSCLC, MSI-H/dMMR tumors, TNBC, or another malignancy that has shown preliminary efficacy following treatment with the study antibody;
Measurable disease per RECIST v1.1 or Lugano Criteria;
   Note: Tumor lesions located in a previously irradiated area or in an area subjected to other loco-regional therapy are considered measurable if clear progression has been demonstrated in the lesion;
Received prior standard therapy for advanced metastatic disease as applicable to tumor type;
Received a maximum of four prior systemic treatment regimens (inclusive of chemotherapy, immunotherapy, and targeted therapy regimens) for advanced or recurrent/metastatic disease;
Received a maximum of one prior anti-PD-1 therapy containing immunotherapy regimen in the advanced/metastatic setting.
   Note: This criterion is not applicable for participants with immunotherapy-naive TNBC in Part 2.
Willingness to undergo pretreatment and on-treatment tumor biopsies to obtain tumor tissue.
   Note: If a participant is scheduled to have a tumor biopsy for the purposes of this study, and it is subsequently determined that tumor tissue cannot safely be obtained, then the participant may still enroll in the study;
Part 2 only: Participants must have evaluable PD-L1 from a fresh tumor biopsy during screening;
Willingness to avoid pregnancy or fathering children based on the criteria below:
a. Men must agree to take appropriate precautions to avoid fathering children (with at least 99% certainty) from screening through 90 days after the last dose of the study antibody and must refrain from donating sperm during this period. Permitted methods that are at least 99% effective in preventing pregnancy should be communicated to the participants and their understanding confirmed;
b. Women of childbearing potential must have a negative serum pregnancy test at screening and before the first dose on Day 1 and must agree to take appropriate precautions to avoid pregnancy (with at least 99% certainty) from screening through 90 days after the last dose of the study antibody. Permitted methods that are at least 99% effective in preventing pregnancy should be communicated to the participants and their understanding confirmed. Women of childbearing potential should refrain from donating oocytes from 30 days prior to the first dose of the study antibody and until 90 days after the last dose of the study antibody;
c. Women of nonchildbearing potential (i.e, surgically sterile with a hysterectomy and/or bilateral oophorectomy OR≥12 months of amenorrhea and at least 50 years of age) are eligible.

Tumor-Specific Criteria for Participants with Histologically or Cytologically Confirmed TNBC
Histologically or cytologically confirmed HER2-negative/ER-negative/PR-negative breast cancer, source documented, defined as both of the following:
a. Estrogen Receptor (ER) and Progesterone Receptor (PgR) negative: <1% of tumor cell nuclei are immunoreactive in the presence of evidence that the sample can express ER or PgR (positive intrinsic controls);
b. Human Epidermal Growth Factor Receptor 2 (HER2) negative as per American Society of Clinical Oncology-College of American Pathologists (ASCO/CAP) guidelines: Immunohistochemistry (IHC) 0 or 1, or Fluorescence In Situ Hybridization (FISH) negative (or equivalent negative test). Subjects with IHC 2 must have a negative by Fluorescence In Situ Hybridization (FISH), (or equivalent negative test);
Unresectable advanced or metastatic TNBC as per current AJCC staging system that is not amenable to local therapy;
Must have received prior treatment with a taxane-containing chemotherapy regimen in the advanced or metastatic setting;
Part 2 participants only: Must not have had prior immunotherapies including PD-1, PD-L1, CTLA-4, or other immune checkpoint inhibitors either alone or as part of a combination therapy regimen.

Tumor-Specific Criteria for Participants with Histologically or Cytologically Confirmed NSCLC
Histologically or cytologically confirmed diagnosis of NSCLC (either nonsquamous or squamous).
a. Documentation of test results for mutations or gene rearrangements for EGFR, ALK, BRAF, and ROS1 if the tumor is of nonsquamous histology only (molecular testing is not currently part of diagnostic guidelines for predominantly squamous histology). If mutations or gene arrangements are present, participant must have progressed on or be intolerable to targeted therapy;
Unresectable advanced or metastatic NSCLC as per current AJCC staging system that is not amenable to local therapy;
Part 2 participants only: Must have documented progression on anti-PD-1 therapy as defined by meeting one of the criteria below:
a. Primary refractory: Must have received prior anti-PD-1 therapy (alone or as part of a combination) in the advanced or metastatic setting for a minimum of 12 weeks and have PD as their best response to treatment;
b. Secondary resistance: Must have received prior anti-PD-1 therapy (alone or as part of a combination) in the advanced or metastatic setting and achieved CR, PR, or SD but later had confirmed PD while receiving anti-PD-1 therapy (PD confirmed at least 4 weeks [no less than 28 days] later).

Tumor-Specific Criteria for Participants with Solid Tumor Indication of Confirmed MSI-H or dMMR Status
MSI-H or dMMR solid tumor, as determined by a local laboratory using IHC or polymerase chain reaction methods and must also have tissue available for central confirmation of diagnosis;
Part 2 participants only who are considered primary refractory to anti-PD-1 therapy are defined as follows: received prior anti-PD-1 therapy (alone or as part of a combination) in the advanced or metastatic setting for a minimum of 12 weeks and have documented PD as their best response to treatment.

Results

Several patients enrolled in the study. Clinical activity was observed in at least a patient subpopulation with MSI-high endometrial cancer. In this subpopulation, a 40 year old patient diagnosed with MSI-high endometrial cancer, having a PD-L1 expression of 10-50%, received adjuvant treatment with carboplatin+taxol, cisplatin+5FU, epirubicine/ciclofosfamide, tamoxifen, and adriamicin, prior to treatment with the study drug. The total sum of target lesions was 51 mm at the start of treatment. The study drug was administered at 75 mg. The total sum of target lesions was reduced to 42 mm after 8 weeks of treatment.

Clinical activity was also observed in a patient population with glioblastoma. In this subpopulation, a 35 year old patient diagnosed with glioblastoma received radiotherapy, surgery, and temozolomide prior to treatment with the study drug. The study drug was administered at 25 mg. The patient showed stable disease, with baseline target lesion dimensions of 9.88 mm×14.62 mm being reduced to 4.87 mm×6.01 mm after 19 months of treatment.

Clinical activity was further observed in a patient population with cervical cancer. In this subpopulation, a 70 year old patient diagnosed with cervical cancer, having a PD-L1 expression of 1% on tumor cells and 30% on tumor immune cells, received prior treatment with radiotherapy, surgery, carboplatin, taxol and avastin, prior to treatment with the study drug. The total sum of target lesions was 40 mm at the start of treatment. The study drug was administered at 50 mg. The total sum of target lesions was reduced to 26 mm after 16 weeks of treatment. This patient achieved a partial response (35% decrease) per RECIST v1.1 as determined by the investigator even after discontinuation from therapy with the study drug.

SEQUENCES

SEQ ID NO: 1: Heavy chain variable region
QVQLVQSGSELKKPGASVKVSCKASGYTFTNFAMNWVRRAPGQGLEWMGWINTNTGN
PTYAQGFTGRFVFSLDTSVNTAYLQISSLKAEDTAVYYCARDWGVIGGHYMDVWGKGTT
VTVSS
SEQ ID NO: 2: HCDR1 according to Kabat from SEQ ID NO: 1
NFAMN
SEQ ID NO: 3: HCDR2 according to Kabat from SEQ ID NO: 1
WINTNTGNPTYAQGFTG
SEQ ID NO: 4: HCDR3 according to Kabat from SEQ ID NO: 1
DWGVIGGHYMDV
SEQ ID NO: 5 Heavy chain variable region
QVQLVQSGAEVKKPGASVKVSCKASGYTFTSYGISWVRQAPGQGLEWMGWISAYNGNT
NYAQKLQGRVTMTTDTSTSTAYMELRSLRSDDTAVYYCARDSDGYGPKAFDYWGQGTL
VTVSS
SEQ ID NO: 6: HCDR1 according to Kabat
SYGIS
SEQ ID NO: 7: HCDR2 according to Kabat
WISAYNGNTNYAQKLQG
SEQ ID NO: 8: HCDR3 according to Kabat
DSDGYGPKAFDY
SEQ ID NO: 9 Heavy chain variable region
EVQLVQSGAEVKKPGESLKISCKGSGYSFTSYWIGWVRQMPGKGLEWMGIIYPDDSDTR
YSPSFQGQVTISADKSSSTAYLQWSSLKASDTAMYYCASFYTGIVGATGAFDVWGQGTTV
TVSS
SEQ ID NO: 10: HCDR1 according to Kabat
SYWIG
SEQ ID NO: 11: HCDR2 according to Kabat
IIYPDDSDTRYSPSFQG
SEQ ID NO: 12: HCDR3 according to Kabat
FYTGIVGATGAFDV
SEQ ID NO: 13 Heavy chain variable region
QVQLVQSGAEVKKPGSSVKVSCKASGGTFSSDAISWVRQAPGQGLEWMGGMIPILGTAN
YAQKFQGRVTITADRSTSTAYMELSSLRSEDTAVYYCVRGATYYYGSGTYYSINWFDPWG
QGTLVTVSS
SEQ ID NO: 14: HCDR1 according to Kabat
SDAIS
SEQ ID NO: 15: HCDR2 according to Kabat
GMIPILGTANYAQKFQG
SEQ ID NO: 16: HCDR3 according to Kabat
GATYYYGSGTYYSINWFDP
SEQ ID NO: 17 Heavy chain variable region
QVQLVQSGSELKKPGASVKVSCRASGYTFTNFAMTWVRQAPGQGPEYMGWINTNTGNP
TYAQGFTGRFVFSLDTSVNTAYLQISSLKAEDTAVYYCARDWASVMVRGDLDYWGQGTL
VTVSS
SEQ ID NO: 18: HCDR1 according to Kabat
NFAMT
SEQ ID NO: 19: HCDR3 according to Kabat
DWASVMVRGDLDY
SEQ ID NO: 20 Heavy chain variable region
QVQLVQSGAEVKKPGASVKVSCKVSGYTLSELSIHWVRQAPGKGVEWMGGFYPEDVEPI
YARKFQGRVTMTEDTSTDTAYMELNSLRSEDTAVYYCAAEGFDNYGSGIRGNWFDPWG
QGTLVTVSS
SEQ ID NO: 21: HCDR1 according to Kabat
ELSIH
SEQ ID NO: 22: HCDR2 according to Kabat
GFYPEDVEPIYARKFQG
SEQ ID NO: 23: HCDR3 according to Kabat
EGFDNYGSGIRGNWFDP
SEQ ID NO: 24 Heavy chain variable region
EVQLVQSGAEVKKPGASVKVSCKVSGYTLTELSMHWVRQSPGKGLEWMGSFYPEDGET
IYAQKFQGRITMTEDTSADTAYMELSSLRSEDTAVYYCATEGVGVIRGNWFDPWGQGTL
VTVSS
SEQ ID NO: 25: HCDR1 according to Kabat
ELSMH
SEQ ID NO: 26: HCDR2 according to Kabat
SFYPEDGETIYAQKFQG
SEQ ID NO: 27: HCDR3 according to Kabat
EGVGVIRGNWFDP
SEQ ID NO: 28 Heavy chain variable region
EVQLVQSGAEVKKPGESLKISCKGSGYSFTSYWIGWVRQMPGKGLEWMGIIFPDDSDTR
YSPSFQGQVTISADKSISTAYLQWSSLKPSDTAMYYCVRLGGYSGYAEDFVDFWGQGTLV
TVSS
SEQ ID NO: 29: HCDR2 according to Kabat
IIFPDDSDTRYSPSFQG
SEQ ID NO: 30: HCDR3 according to Kabat
LGGYSGYAEDFVDF
SEQ ID NO: 31 Heavy chain variable region
EVQLVQSGAEVKKPGASVKVSCKVSGYTLTKLSMHWVRQAPGKGLEWMGGFEPEDGE
TINAQKFQGRVTMTEDTSTDTAYMELSSLRSEDTAVYYCATDLRLGASYYYSYMDVWGR
GTMVTVSS
SEQ ID NO: 32: HCDR1 according to Kabat
KLSMH
SEQ ID NO: 33: HCDR2 according to Kabat
GFEPEDGETINAQKFQG
SEQ ID NO: 34: HCDR3 according to Kabat
DLRLGASYYYSYMDV
SEQ ID NO: 35 Heavy chain variable region
QITLKESGPTLVKPTQTLTLSCTFSGFSLSTSGMSVGWIRQPPGKALEWLALIYWNDDKY
FSPSLKSRLTITKDTSKNQVVLTLTNMDPVDTATYYCAHTLWGSDDVFDVWGQGTMVT
VSS
SEQ ID NO: 36: HCDR1 according to Kabat
TSGMSVG
SEQ ID NO: 37: HCDR2 according to Kabat
LIYWNDDKYFSPSLKS
SEQ ID NO: 38: HCDR3 according to Kabat
TLWGSDDVFDV
SEQ ID NO: 39 Heavy chain variable region
EVQLVQSGAEVKKPGESLKISCKVSGYSFTNYWIGWVRQMPGKGLEWMGIIYPGDSDTR
YSPSFQGQVTISADKSISTAYLQWHTLKASDTAMYYCARHQGYSFSGSHIDDYWGQGTL
VTVSS
SEQ ID NO: 40: HCDR1 according to Kabat
NYWIG
SEQ ID NO: 41: HCDR2 according to Kabat
IIYPGDSDTRYSPSFQG
SEQ ID NO: 42: HCDR3 according to Kabat
HQGYSFSGSHIDDY
SEQ ID NO: 43 Heavy chain variable region
EVQLVQSGAEVRKPGESLKISCKGSGYSFTTYWIGWVRQMPGKGLEWMGIIYPGDSDTR
YSPSFQGQVTISADKSISTVYLQWSSLKASDTAMYYCARHAGFIITSQNIDDYWGQGTLVT
VSS
SEQ ID NO: 44: HCDR1 according to Kabat
TYWIG
SEQ ID NO: 41: HCDR2 according to Kabat
IIYPGDSDTRYSPSFQG
SEQ ID NO: 45: HCDR3 according to Kabat
HAGFIITSQNIDDY
SEQ ID NO: 46 Heavy chain variable region
EVQLVQSGSELKKPGASVKVSCKASGYTFTNFAMNWVRQAPGQGLEWMGWINTNTGN
PTYAQDFTGRFVFSLDTSGNTAYLQISSLKAEDTAVYYCARDWGLVAIGYFDYWGQGTL
VTVSS
SEQ ID NO: 47: HCDR2 according to Kabat
WINTNTGNPTYAQDFTG
SEQ ID NO: 48: HCDR3 according to Kabat
DWGLVAIGYFDY
SEQ ID NO: 49 Heavy chain variable region
QITLKESGPTLVKPTQTLTLTCTFSGFSLSTTGVGVNWIRQPPGEALEWLALIYWNDDTY
YSPSLKSRLTITKDTSKNQVVLTMTNMDPVDTATYYCAHEGIIGFLGGNWFDPWGQGTL
VTVSS
SEQ ID NO: 50: HCDR1 according to Kabat
TTGVGVN
SEQ ID NO: 51: HCDR2 according to Kabat
LIYWNDDTYYSPSLKS
SEQ ID NO: 52: HCDR3 according to Kabat
EGIIGFLGGNWFDP
SEQ ID NO: 53 Heavy chain variable region
QVQLVQSGSELKKPGASVKVSCKASGYTFTSHAMNWVRQAPGQGLEWMGWINPNTGN
PTYAQGFTGRFVFSLDTSVSTAYLQISSLKAEDTAVYYCARDRKYVTNWVFAEDFQHWG
QGTLVTVSS
SEQ ID NO: 54: HCDR1 according to Kabat
SHAMN
SEQ ID NO: 55: HCDR2 according to Kabat
WINPNTGNPTYAQGFTG
SEQ ID NO: 56: HCDR3 according to Kabat
DRKYVTNWVFAEDFQH
SEQ ID NO: 57 Heavy chain variable region
QVQLVQSGSELKKPGASVKVSCKASGYTFTSHAMNWVRQAPGQGLEWMGWINPNTGN
PTYAQGFTGRFVFSLDTSVSTAYLQISSLKAEDTAVYYCAIDRGYMSNWVFAEYFPHWG
QGTLVTVSS
SEQ ID NO: 58: HCDR3 according to Kabat
DRGYMSNWVFAEYFPH
SEQ ID NO: 59 Heavy chain variable region
QVQLVQSGSELKKPGASVKVSCKASGYTFTSYAMNWVRQAPGQGLEWMGWINTNTGN
PTYAQGFTGRFVFSLDTSVSTAYLQISSLKAEDTAVYYCATDRGYISSWVFAEDFQHWGQ
GTLVTVSS
SEQ ID NO: 60: HCDR1 according to Kabat
SYAMN
SEQ ID NO: 61: HCDR3 according to Kabat
DRGYISSWVFAEDFQH
SEQ ID NO: 62 Heavy chain variable region
QVQLVQSGSELKKPGASVKVSCTASGYTFTSYAMNWVRQAPGQRLEWMACVNPNTGSP
TYAQGSTGRFVVSLDTSVSTAYLQISSLKAEDTAVYYCARDRKYVTNWVFAEDFQHWGH
GTLVTVSS
SEQ ID NO: 63: HCDR2 according to Kabat
CVNPNTGSPTYAQGSTG
SEQ ID NO: 64 Heavy chain variable region
QVQLVQSGSELKKPGASVKVSCKASGYTFTNYAMNWVRQAPGQGLEWMGWMNPNTG
NPTYAQGSTGRFVVSLDTSVSTAYLQISSLKAEDTAVYYCARDRKYVTNWVFAEDFQHW
GRGTLVTVSS
SEQ ID NO: 65: HCDR1 according to Kabat
NYAMN
SEQ ID NO: 66: HCDR2 according to Kabat
WMNPNTGNPTYAQGSTG
SEQ ID NO: 67 Heavy chain variable region
QVQLVQSGSELKKPGASVKVSCKASGYTFTNYAINWVRQAPGQGLEWMGWINPNTGNP
TYAQGFTGRFVFSLDTSVSTAYLQISSLKAEDTAVYYCARDRKYVTNWVFAEDFQHWGR
GTLVTVSS
SEQ ID NO: 68: HCDR1 according to Kabat
NYAIN
SEQ ID NO: 69 Heavy chain variable region
EVQLVQSGAEVKKPGSSVKVSCKASGDTFNTYSITWVRQAPGQGLEWMGSIVPIFGTINN
AQKFQGRVTITADKSANTAYMELSSLRSEDTAVYYCARDNTMVRGVDYYYMDVWGKGT
MVTVSS
SEQ ID NO: 70: HCDR1 according to Kabat
TYSIT
SEQ ID NO: 71: HCDR2 according to Kabat
SIVPIFGTINNAQKFQG
SEQ ID NO: 72: HCDR3 according to Kabat
DNTMVRGVDYYYMDV
SEQ ID NO: 73 Heavy chain variable region
EVQLVQSGAEVKKPGSSVKVSCKASGGIFSTYAISWVRQAPGQGLEWMGGIIPIFDTPNY
AQKFQGRVTITADKSTSTAYMDLSSLRSEDTAVYYCAKNVRGYSAYDLDYWGQGTLVTV
SS
SEQ ID NO: 74: HCDR1 according to Kabat
TYAIS
SEQ ID NO: 75: HCDR2 according to Kabat
GIIPIFDTPNYAQKFQG
SEQ ID NO: 76: HCDR3 according to Kabat
NVRGYSAYDLDY
SEQ ID NO: 77 Heavy chain variable region
EVQLVQSGAEVKNPGSSVKVSCKATGGTFNTYGTNWVRQAPGQGLEWMGGIIPIFGTAN
YAQKFQGRVTITADKSTTTAYMEVSSLRSEDTAVYYCARGGADMGTLDYWGQGTLVTVS
S
SEQ ID NO: 78: HCDR1 according to Kabat
TYGTN
SEQ ID NO: 79: HCDR2 according to Kabat
GIIPIFGTANYAQKFQG
SEQ ID NO: 80: HCDR3 according to Kabat
GGADMGTLDY
SEQ ID NO: 81 Heavy chain variable region
EVQLVQSGAEVMRPGSSVKVSCKASGGIFNTYTIIWVRQAPGQGLEWMGGIIPIFDTPNF
AQKFQGRLTITADKSTNTAYMELTSLRSEDTAVYYCAREGCNHGVCYPYWGQGTLVTVS
S
SEQ ID NO: 82: HCDRI according to Kabat
TYTII
SEQ ID NO: 83: HCDR2 according to Kabat
GIIPIFDTPNFAQKFQG
SEQ ID NO: 84: HCDR3 according to Kabat
EGCNHGVCYPY
SEQ ID NO: 85 Heavy chain variable region
QVQLVQSGAEVKKPGSSVKVSCKASGDTFRSYGITWVRQAPGQGLEWMGGIIPIFGTTN
YAQKFQGRVTITADKSTSTVYMELSSLRSEDTAVYYCARRRGYSNPHWLDPWGQGTLVT
VSS
SEQ ID NO: 86: HCDR1 according to Kabat
SYGIT
SEQ ID NO: 87: HCDR2 according to Kabat
GIIPIFGTTNYAQKFQG
SEQ ID NO: 88: HCDR3 according to Kabat
RRGYSNPHWLDP
SEQ ID NO: 89 Heavy chain variable region
QVQLVQSGAEVKKPGSSVKVSCKASGGTESTYGILWVRQAPGQGLEWMGGIIPIFGTAN
YAQKFQGRVTITADISTSTAYMELSSLRSEDTAVYYCARGGGNYYEFVYWGQGTLVTVSS
SEQ ID NO: 90: HCDR1 according to Kabat
TYGIL
SEQ ID NO: 91: HCDR3 according to Kabat
GGGNYYEFVY
SEQ ID NO: 92 Heavy chain variable region
EVQLVQSGAEVKKPGSSVRVSCKASGGTFNTYAINWVRQAPGQGLEWVGRIIPIFDTANY
AQKFQGRVTISADKSTTTAYMELSSLRSEDTAVFYCAKDETGYSSSNFQHWGQGTLVTV
SS
SEQ ID NO: 93: HCDR1 according to Kabat
TYAIN
SEQ ID NO: 94: HCDR2 according to Kabat
RIIPIFDTANYAQKFQG
SEQ ID NO: 95: HCDR3 according to Kabat
DETGYSSSNFQH
SEQ ID NO: 96 Heavy chain variable region
QVQLVQSGSELKKPGASVKVSCKASGYTFTNYAINWVRQAPGQGLEWMGWINPNTGNP
TYAQGFTGRFVFSLDTSVSTAYLQISSLKAEDTAVYYCARDRKYVTNWVFAEDFQHWGQ
GTLVTVSS
SEQ ID NO: 97 Heavy chain variable region
QVQLVQSGAEVKRPGSSVKVSCKASGGTFNTYSITWVRQAPGQGLEWMGGIIPVFGTSK
YAQKFQDRVTITADKSTNTAYMELSSLRSEDTAVYYCARDPSFSSSSGWFDPWGQGTLV
TVSS
SEQ ID NO: 98: HCDR2 according to Kabat
GIIPVFGTSKYAQKFQD
SEQ ID NO: 99: HCDR3 according to Kabat
DPSFSSSSGWFDP
SEQ ID NO: 100 Heavy chain variable region
QVQLVQSGAEVKKPGSSVKVSCKASGGTFNTYAINWVRQAPGQGLEWMGGIIPIFDTAN
YAQRFQGRVTITADKSTSTAYMELSSLRSEDTAVYFCAKDQTGYSSTLFDYWGQGTLVTV
SS
SEQ ID NO: 101: HCDR2 according to Kabat
GIIPIFDTANYAQRFQG
SEQ ID NO: 102: HCDR3 according to Kabat
DQTGYSSTLFDY
SEQ ID NO: 103 Heavy chain variable region
QVQLVQSGSELKKPGASVKVSCKASGYTFTSHAMNWVRQAPGQGLEWMGWINPNTGN
PTYAQGFTGRFVFSLDTSVSTAYLQISSLKAEDTAVYYCAIDRGYMSNWVFAEYFPHWG
QGTLVTVSS
SEQ ID NO: 104 Heavy chain variable region
EVQLVQSGAEVKKPGSSVKVSCKASGGTFSTYAISWVRQAPGQGLEWMGWIIPIFDTGN
YAQKIQGRVTITADKSTSTAYMELTSLRSEDTAVYYCARHDYTNTVDAFDIWGQGTMVT
VSS
SEQ ID NO: 105: HCDR2 according to Kabat
WIIPIFDTGNYAQKIQG
SEQ ID NO: 106: HCDR3 according to Kabat
HDYTNTVDAFDI
SEQ ID NO: 107 Heavy chain variable region
QVQLVQSGAEVKKPGSSVKVSCKASGDTFRSYGITWVRQAPGQGLEWMGGIIPVFGTTN
YAQKFQGRVTITADKSTSTVFMELNSLRSEDTAVYYCARRRGYSNPHWLDPWGQGTLVT
VSS
SEQ ID NO: 108: HCDR2 according to Kabat
GIIPVFGTTNYAQKFQG
SEQ ID NO: 109: Amino acid sequence of human common light chain IGKV1-39/jk1
DIQMTQSPSSLSASVGDRVTITCRASQSISSYLNWYQQKPGKAPKLLIYAASSLQSGVPSR
FSGSGSGTDFTLTISSLQPEDFATYYCQQSYSTPPTFGQGTKVEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
SEQ ID NO: 110: Amino acid sequence of common light chain variable domain
DIQMTQSPSSLSASVGDRVTITCRASQSISSYLNWYQQKPGKAPKLLIYAASSLQSGVPSR
FSGSGSGTDFTLTISSLQPEDFATYYCQQSYSTPPTFGQGTKVEIK
SEQ ID NO: 111: Amino acid sequence of common light chain constant domain
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSENRGEC
SEQ ID NO: 112: Amino acid sequence of CH1
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRV
SEQ ID NO: 113: Amino acid sequence of the hinge
EPKSCDKTHTCPPCP
SEQ ID NO: 114: Amino acid sequence of CH2
APELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAK
SEQ ID NO: 115: Amino acid sequence of CH3 with KK mutations
GQPREPQVYTKPPSREEMTKNQVSLKCLVKGFYPSDIAVEWESNGQPENNYKTTPPVLD
SDGSFFLYSKLTVDKSRWQQGNVFSCSVMHEALHNHYTQKSLSLSPGK
SEQ ID NO: 116: Amino acid sequence of CH3 with DE mutations
GQPREPQVYTDPPSREEMTKNQVSLTCEVKGFYPSDIAVEWESNGQPENNYKTTPPVLD
SDGSFFLYSKLTVDKSRWQQGNVFSCSVMHEALHNHYTQKSLSLSPGK
SEQ ID NO: 117: Amino acid sequence of CD137
MGNSCYNIVATLLLVLNFERTRSLQDPCSNCPAGTFCDNNRNQICSPCPPNSFS
SAGGQRTCDICRQCKGVFRTRKECSSTSNAECDCTPGFHCLGAGCSMCEQDCK
QGQELTKKGCKDCCFGTFNDQKRGICRPWTNCSLDGKSVLVNGTKERDWCG
PSPADLSPGASSVTPPAPAREPGHSPQIISFFLALTSTALLFLLFFLTLRFSWKR
GRKKLLYIFKQPFMRPVQTTQEEDGCSCRFPEEEEGGCEL

1. A multispecific antibody that comprises an antigen binding site that binds an extracellular part of CD137 and an antigen binding site that binds an extracellular part of a second membrane protein for use in a method of treatment of cancer in a subject in need thereof, wherein the multispecific antibody is administered in a dose of between 25-300 mg, preferably between 25-150 mg or 25-100 mg, more preferably between 50-100 mg.

2. A method of treating cancer in a subject in need thereof, the method comprising administering between 25-300 mg, preferably between 25-150 mg or 25-100 mg, more preferably between 50-100 mg, of a multispecific antibody that comprises an antigen binding site that binds an extracellular part of CD137 and an antigen binding site that binds an extracellular part of a second membrane protein to the subject in need thereof.

3. The multispecific antibody for use according to claim 1 or method according to claim 2, wherein the cancer is selected from:
any solid tumor harboring MSI-high alterations;
cervical cancer, in particular PD-L1 positive cervical cancer, more in particular cervical cancer with high expression of PD-L1;
endometrial cancer, in particular MSI-high endometrial cancer;
lung cancer, in particular non-small cell lung cancer (NSCLC), more in particular PD-L1 positive NSCLC, more in particular NSCLC with high expression of PD-L1;
brain cancer, in particular glioblastoma; and
breast cancer, in particular metastatic breast cancer and triple negative breast 25 cancer (TNBC).

4. The multispecific antibody for use according to claim 1 or method according to claim 2, wherein the cancer is selected from:
any solid tumor harboring MSI-high alterations;
endometrial cancer, in particular MSI-high endometrial cancer;
lung cancer, in particular non-small cell lung cancer (NSCLC), more in particular NSCLC with high expression of PD-L1; and
breast cancer, in particular metastatic breast cancer and triple negative breast cancer (TNBC).

5. A multispecific antibody that comprises an antigen binding site that binds an extracellular part of CD137 and an antigen binding site that binds an extracellular part of a second membrane protein for use in a method of treatment of cancer in a subject in need thereof, wherein the cancer is selected from:
any solid tumor harboring Microsatellite Instability-High (MSI-high) alterations;
cervical cancer, in particular PD-L1 positive cervical cancer, more in particular cervical cancer with high expression of PD-L1;
endometrial cancer, in particular MSI-high endometrial cancer;
lung cancer, in particular non-small cell lung cancer (NSCLC), more in particular PD-L1 positive NSCLC, more in particular NSCLC with high expression of PD-L1;
brain cancer, in particular glioblastoma; and
breast cancer, in particular metastatic breast cancer and triple negative breast cancer (TNBC), and
wherein the multispecific antibody is administered in a dose of between 10-1200 mg, preferably between 25-600 or mg 25-300 mg, more preferably between 25-150 mg or 25-100 mg or 25-75 mg, most preferably between 25-50 mg or 50-100 mg.

6. The multispecific antibody for use according to claim 5, wherein the cancer is selected from:
any solid tumor harboring Microsatellite Instability-High (MSI-high) alterations;
endometrial cancer, in particular MSI-high endometrial cancer;
lung cancer, in particular non-small cell lung cancer (NSCLC), more in particular NSCLC with high expression of PD-L1; and
breast cancer, in particular metastatic breast cancer and triple negative breast cancer (TNBC), and
wherein the multispecific antibody is administered in a dose of between 10-1200 mg, preferably between 25-600 or mg 25-300 mg, more preferably between 25-150 mg or 25-100 mg, most preferably between 50-100 mg.

7. A method of treating cancer in a subject in need thereof, the method comprising administering between 10-1200 mg, preferably between 25-600 or mg 25-300 mg, more preferably between 25-150 mg or 25-100 mg or 25-75 mg, most preferably between 25-50 mg or 50-100 mg, of a multispecific antibody that comprises an antigen binding site that binds an extracellular part of CD137 and an antigen binding site that binds an extracellular part of a second membrane protein to a subject having cancer, wherein the cancer is selected from:
any solid tumor harboring Microsatellite Instability-High (MSI-high) alterations;
cervical cancer, in particular PD-L1 positive cervical cancer, more in particular cervical cancer with high expression of PD-L1;
endometrial cancer, in particular MSI-high endometrial cancer;
lung cancer, in particular non-small cell lung cancer (NSCLC), more in particular PD-L1 positive NSCLC, more in particular NSCLC with high expression of PD-L1;
brain cancer, in particular glioblastoma; and
breast cancer, in particular metastatic breast cancer or triple negative breast cancer (TNBC).

8. The method according to claim 7, the method comprising administering between 10-1200 mg, preferably between 25-600 or mg 25-300 mg, more preferably between 25-150 mg or 25-100 mg, most preferably between 50-100 mg, of a multispecific antibody that comprises an antigen binding site that binds an extracellular part of CD137 and an antigen binding site that binds an extracellular part of a second membrane protein to a subject having cancer, wherein the cancer is selected from:
any solid tumor harboring Microsatellite Instability-High (MSI-high) alterations;
endometrial cancer, in particular MSI-high endometrial cancer;
lung cancer, in particular non-small cell lung cancer (NSCLC), more in particular NSCLC with high expression of PD-L1; and
breast cancer, in particular metastatic breast cancer or triple negative breast cancer (TNBC).

9. The multispecific antibody for use or method according to any one of claims 1-8, wherein the multispecific antibody is administered intravenously.

10. The multispecific antibody for use or method according to any one of claims 1-9, wherein the multispecific antibody is administered once every two weeks.

11. The multispecific antibody for use or method according to any one of claims 1-10, wherein said second membrane protein is not a member of the TNF receptor superfamily.

12. The multispecific antibody for use or method according to any one of claims 1-11, wherein said second membrane protein is a member of the B7 family.

13. The multispecific antibody for use or method according to any one of claims 1-12 wherein the second membrane protein is PD-L1 or PD-L2, preferably PD-L1.

14. The multispecific antibody for use or method according to any one of claims 1-13, wherein the multispecific antibody comprises one antigen binding site that binds the PD-1 binding domain of PD-L1.

15. The multispecific antibody for use or method according to any one of claims 1-14, wherein the multispecific antibody comprises one antigen binding site that binds the CD137L binding domain of CD137.

16. The multispecific antibody for use or method according to any one of claims 1-15, wherein the multispecific antibody comprises one antigen binding site that blocks the binding of a ligand to CD137 or binds an extracellular ligand-blocking binding site of CD137, preferably a CD137L blocking binding site.

17. The multispecific antibody for use or method according to any one of claims 1-16, wherein the variable domain that binds an extracellular part of CD137 is defined as a variable domain that, when in a bivalent monospecific antibody format that comprises two of said variable domains that bind CD137, does not stimulate activity of CD137 on a cell or does so at a reduced level in comparison to one of said variable domain as part of a bispecific antibody having a second variable domain binding a tumor associated antigen, preferably a member of the B7 family, more preferably PD-L1.

18. The multispecific antibody for use or method according to any one of claims 1-17, wherein the variable domain that binds an extracellular part of CD137 is capable of stimulating activity of CD137 on a cell when combined in a multispecific antibody with a second variable domain which binds to PD-L1, when the multispecific antibody is in the presence of a first cell expressing CD137 and a second cell expressing PD-L1.

19. The multispecific antibody for use or method according to any one of claims 1-18, wherein the multispecific antibody is capable of binding CD137 and PD-L1, preferably simultaneously.

20. The multispecific antibody for use or method according to any one of claims 1-19, wherein the multispecific antibody only induces or activates CD137 signaling in the presence of PD-L1 expressing cells.

21. The multispecific antibody for use or method according to any one of claims 1-20, wherein the antigen binding sites of said multispecific antibody consist of one immunoglobulin variable domain that binds CD137 and one immunoglobulin variable domain that binds the extracellular part of a second membrane protein.

22. The multispecific antibody for use or method according to any one of claims 1-21, wherein said multispecific antibody is a full length antibody.

23. The multispecific antibody for use or method according to any one of claims 1-22, wherein said multispecific antibody is an IgG1 molecule without Fe effector function.

24. The multispecific antibody for use or method according to any one of claims 1-23, wherein the second membrane protein is not to a significant extent expressed by a T-cell.

25. The multispecific antibody for use or method according to any one of claims 1-24, wherein said second membrane protein is present on the cell membrane as a part of a multimeric membrane protein comprising two or more of said second membrane proteins.

26. The multispecific antibody for use or method according to any one of claims 1-25, wherein said second membrane protein is present on the cell membrane as a part of a homodimer or a homotrimer.

27. The multispecific antibody for use or method according to any one of claims 1-26, wherein the antibody comprises a heavy chain variable region that binds an extracellular part of CD137 comprising a CDR3 region having an amino acid sequence as set forth in SEQ ID NO: 23; SEQ ID NO: 27; SEQ ID NO: 34 or SEQ ID NO: 52, or variants thereof.

28. The multispecific antibody for use or method according to any one of claims 1-27, wherein the antibody comprises a heavy chain variable region that binds an extracellular part of CD137 comprising a CDR2 region having an amino acid sequence as set forth in SEQ ID NO: 22; SEQ ID NO: 26; SEQ ID NO: 33; or SEQ ID NO: 51, or variants thereof.

29. The multispecific antibody for use or method according to any one of claims 1-27, wherein the antibody comprises a heavy chain variable region that binds an extracellular part of CD137 comprising a CDR1 region having an amino acid sequence as set forth in SEQ ID NO: 21; SEQ ID NO: 25; SEQ ID NO: 32; or SEQ ID NO: 50, or variants thereof.

30. The multispecific antibody for use or method according to any one of claims 1-29, wherein the antibody comprises a heavy chain variable region that binds an extracellular part of CD137 having an amino acid sequence as set forth in SEQ ID NO: 1; SEQ ID NO: 5; SEQ ID NO: 9; SEQ ID NO: 13; SEQ ID NO: 17; SEQ ID NO: 20; SEQ ID NO: 24; SEQ ID NO: 28; SEQ ID NO: 31; SEQ ID NO: 35; SEQ ID NO: 39; SEQ ID NO: 43; SEQ ID NO: 46; or SEQ ID NO: 49, preferably SEQ ID NO: 20; SEQ ID NO: 24; SEQ ID NO: 31; or SEQ ID NO: 49, or variants thereof.

31. The multispecific antibody for use or method according to any one of claims 1-30, wherein the antibody comprises a heavy chain variable region that binds an extracellular part of PD-L1 comprising a CDR3 region having an amino acid sequence as set forth in SEQ ID NO: 56; SEQ ID NO: 58; SEQ ID NO: 61; SEQ ID NO: 84; SEQ ID NO: 88; SEQ ID NO: 91; SEQ ID NO: 95; SEQ ID NO: 102; or SEQ ID NO: 106, preferably SEQ ID NO: 56; SEQ ID NO: 91; SEQ ID NO: 95; or SEQ ID NO: 102, or variants thereof.

32. The multispecific antibody for use or method according to any one of claims 1-31, wherein the antibody comprises a heavy chain variable region that binds an extracellular part of PD-L1 comprising a CDR2 region having an amino acid sequence as set forth in SEQ ID NO: 3; SEQ ID NO: 55; SEQ ID NO: 63; SEQ ID NO: 66; SEQ ID NO: 79; SEQ ID NO: 83; SEQ ID NO: 87; SEQ ID NO: 94; SEQ ID NO: 101; or SEQ ID NO: 105, or variants thereof.

33. The multispecific antibody for use or method according to any one of claims 1-32, wherein the antibody comprises a heavy chain variable region that binds an extracellular part of PD-L1 comprising a CDR1 region having an amino acid sequence as set forth in SEQ ID NO: 54; SEQ ID NO: 60; SEQ ID NO: 65; SEQ ID NO: 68; SEQ ID NO: 74; SEQ ID NO: 82; SEQ ID NO: 86; SEQ ID NO: 90; or SEQ ID NO: 93, or variants thereof.

34. The multispecific antibody for use or method according to any one of claims 1-33, wherein the antibody comprises a heavy chain variable region that binds an extracellular part of PD-L1 having an amino acid sequence as set forth in SEQ ID NO: 53; SEQ ID NO: 57; SEQ ID NO: 59; SEQ ID NO: 62; SEQ ID NO: 64; SEQ ID NO: 67; SEQ ID NO: 69; SEQ ID NO: 73; SEQ ID NO: 77; SEQ ID NO: 81; SEQ ID NO: 85; SEQ ID NO: 89; SEQ ID NO: 92; SEQ ID NO: 96; SEQ ID NO: 97; SEQ ID NO: 100; SEQ ID NO: 103; SEQ ID NO: 104; SEQ ID NO: 107, preferably SEQ ID NO: 67, SEQ ID NO: 89, SEQ ID NO: 92, or SEQ ID NO: 100, or variants thereof.

35. The multispecific antibody for use or method according to any one of claims 1-34, wherein the multispecific antibody comprises CDR 1, 2 and 3 of MF6797 and CDRs 1, 2 and 3 of MF7702, or variants thereof.

36. The multispecific antibody for use or method according to any one of claims 1-34, wherein the multispecific antibody comprises SEQ ID NO. 49 and SEQ ID NO. 67, or variants thereof.

37. A multispecific antibody for use in a method of treatment of cancer in a subject in need thereof, wherein the antibody comprises a binding domain that binds CD137 comprising:
a variable domain that comprises a CDR1 having an amino acid sequence as set forth in SEQ ID NO: 50, a CDR2 having an amino acid sequence as set forth in SEQ ID NO: 51 and a CDR3 having an amino acid sequence as set forth in SEQ ID NO: 52; or
a variable domain that comprises a CDR1 having an amino acid sequence as set forth in SEQ ID NO: 40, a CDR2 having an amino acid sequence as set forth in SEQ ID NO: 41 and a CDR3 having an amino acid sequence as set forth in SEQ ID NO: 42; or
a variable domain that comprises a CDR1 having an amino acid sequence as set forth in SEQ ID NO: 21, a CDR2 having an amino acid sequence as set forth in SEQ ID NO: 22 and a CDR3 having an amino acid sequence as set forth in SEQ ID NO: 23; or
a variable domain that comprises a CDR1 having an amino acid sequence as set forth in SEQ ID NO: 32, a CDR2 having an amino acid sequence as set forth in SEQ ID NO: 33 and a CDR3 having an amino acid sequence as set forth in SEQ ID NO: 34; and/or
wherein the antibody comprises a binding domain that binds PD-L1, comprising:
a variable domain that comprises a CDR1 having an amino acid sequence as set forth in SEQ ID NO: 68, a CDR2 having an amino acid sequence as set forth in SEQ ID NO: 55 and a CDR3 having an amino acid sequence as set forth in SEQ ID NO: 56; or
a variable domain that comprises a CDR1 having an amino acid sequence as set forth in SEQ ID NO: 93, a CDR2 having an amino acid sequence as set forth in SEQ ID NO: 94 and a CDR3 having an amino acid sequence as set forth in SEQ ID NO: 95; or
a variable domain that comprises a CDR1 having an amino acid sequence as set forth in SEQ ID NO: 93, a CDR2 having an amino acid sequence as set forth in SEQ ID NO: 101 and a CDR3 having an amino acid sequence as set forth in SEQ ID NO: 102; or
a variable domain that comprises a CDR1 having an amino acid sequence as set forth in SEQ ID NO: 90, a CDR2 having an amino acid sequence as set forth in SEQ ID NO: 79 and a CDR3 having an amino acid sequence as set forth in SEQ ID NO: 91,
each of the individual SEQ ID NOs having 0, 1, 2, 3, 4 or 5 amino acid insertions, deletions, or substitutions, or a combination thereof,
wherein the multispecific antibody is administered in a dose of between 25-300 mg, preferably between 25-150 or mg 25-100 mg or 25-75 mg, more preferably between 25-50 mg or 50-100 mg.

38. The multispecific antibody for use according to claim 37, wherein the multispecific antibody is administered in a dose of between 25-300 mg, preferably between 25-150 or mg 25-100 mg, more preferably between 50-100 mg.

39. A method of treating cancer in a subject in need thereof, the method comprising administering between 25-300 mg, preferably between 25-150 or mg 25-100 mg or 25-75 mg, more preferably between 25-50 mg or 50-100 mg, of a multispecific antibody that comprises a binding domain that binds CD137, comprising:
a variable domain that comprises a CDR1 having an amino acid sequence as set forth in SEQ ID NO: 50, a CDR2 having an amino acid sequence as set forth in SEQ ID NO: 51 and a CDR3 having an amino acid sequence as set forth in SEQ ID NO: 52; or
a variable domain that comprises a CDR1 having an amino acid sequence as set forth in SEQ ID NO: 40, a CDR2 having an amino acid sequence as set forth in SEQ ID NO: 41 and a CDR3 having an amino acid sequence as set forth in SEQ ID NO: 42; or
a variable domain that comprises a CDR1 having an amino acid sequence as set forth in SEQ ID NO: 21, a CDR2 having an amino acid sequence as set forth in SEQ ID NO: 22 and a CDR3 having an amino acid sequence as set forth in SEQ ID NO: 23; or
a variable domain that comprises a CDR1 having an amino acid sequence as set forth in SEQ ID NO: 32, a CDR2 having an amino acid sequence as set forth in SEQ ID NO: 33 and a CDR3 having an amino acid sequence as set forth in SEQ ID NO: 34; and/or
wherein the antibody comprises a binding domain that binds PD-L1, comprising:
a variable domain that comprises a CDR1 having an amino acid sequence as set forth in SEQ ID NO: 68, a CDR2 having an amino acid sequence as set forth in SEQ ID NO: 55 and a CDR3 having an amino acid sequence as set forth in SEQ ID NO: 56; or
a variable domain that comprises a CDR1 having an amino acid sequence as set forth in SEQ ID NO: 93, a CDR2 having an amino acid sequence as set forth in SEQ ID NO: 94 and a CDR3 having an amino acid sequence as set forth in SEQ ID NO: 95; or
a variable domain that comprises a CDR1 having an amino acid sequence as set forth in SEQ ID NO: 93, a CDR2 having an amino acid sequence as set forth in SEQ ID NO: 101 and a CDR3 having an amino acid sequence as set forth in SEQ ID NO: 102; or
a variable domain that comprises a CDR1 having an amino acid sequence as set forth in SEQ ID NO: 90, a CDR2 having an amino acid sequence as set forth in SEQ ID NO: 79 and a CDR3 having an amino acid sequence as set forth in SEQ ID NO: 91,
each of the individual SEQ ID NOs having 0, 1, 2, 3, 4 or 5 amino acid insertions, deletions, or substitutions, or a combination thereof.

40. The method according to claim 39, wherein the multispecific antibody is administered in a dose of between 25-300 mg, preferably between 25-150 or mg 25-100 mg, more preferably between 50-100 mg.

41. The multispecific antibody for use or method according to any one of claims 37-40, wherein the cancer is selected from:
any solid tumor harboring MSI-high alterations;
cervical cancer, in particular PD-L1 positive cervical cancer, more in particular cervical cancer with high expression of PD-L1;
endometrial cancer, in particular MSI-high endometrial cancer;
lung cancer, in particular non-small cell lung cancer (NSCLC), more in particular PD-L1 positive NSCLC, more in particular NSCLC with high expression of PD-L1;
brain cancer, in particular glioblastoma; and
breast cancer, in particular metastatic breast cancer and triple negative breast cancer (TNBC).

42. The multispecific antibody for use or method according to any one of claims 37-40, wherein the cancer is selected from:
any solid tumor harboring MSI-high alterations;
endometrial cancer, in particular MSI-high endometrial cancer;
lung cancer, in particular non-small cell lung cancer (NSCLC), more in particular NSCLC with high expression of PD-L1; and
breast cancer, in particular metastatic breast cancer and triple negative breast cancer (TNBC).

43. A multispecific antibody for use in a method of treatment of cancer in a subject in need thereof, wherein the antibody comprises a binding domain that binds CD137 comprising:
a variable domain that comprises a CDR1 having an amino acid sequence as set forth in SEQ ID NO: 50, a CDR2 having an amino acid sequence as set forth in SEQ ID NO: 51 and a CDR3 having an amino acid sequence as set forth in SEQ ID NO: 52; or
a variable domain that comprises a CDR1 having an amino acid sequence as set forth in SEQ ID NO: 40, a CDR2 having an amino acid sequence as set forth in SEQ ID NO: 41 and a CDR3 having an amino acid sequence as set forth in SEQ ID NO: 42; or
a variable domain that comprises a CDR1 having an amino acid sequence as set forth in SEQ ID NO: 21, a CDR2 having an amino acid sequence as set forth in SEQ ID NO: 22 and a CDR3 having an amino acid sequence as set forth in SEQ ID NO: 23; or
a variable domain that comprises a CDR1 having an amino acid sequence as set forth in SEQ ID NO: 32, a CDR2 having an amino acid sequence as set forth in SEQ ID NO: 33 and a CDR3 having an amino acid sequence as set forth in SEQ ID NO: 34; and/or
wherein the antibody comprises a binding domain that binds PD-L1, comprising:
a variable domain that comprises a CDR1 having an amino acid sequence as set forth in SEQ ID NO: 68, a CDR2 having an amino acid sequence as set forth in SEQ ID NO: 55 and a CDR3 having an amino acid sequence as set forth in SEQ ID NO: 56; or
a variable domain that comprises a CDR1 having an amino acid sequence as set forth in SEQ ID NO: 93, a CDR2 having an amino acid sequence as set forth in SEQ ID NO: 94 and a CDR3 having an amino acid sequence as set forth in SEQ ID NO: 95; or
a variable domain that comprises a CDR1 having an amino acid sequence as set forth in SEQ ID NO: 93, a CDR2 having an amino acid sequence as set forth in SEQ ID NO: 101 and a CDR3 having an amino acid sequence as set forth in SEQ ID NO: 102; or
a variable domain that comprises a CDR1 having an amino acid sequence as set forth in SEQ ID NO: 90, a CDR2 having an amino acid sequence as set forth in SEQ ID NO: 79 and a CDR3 having an amino acid sequence as set forth in SEQ ID NO: 91,
each of the individual SEQ ID NOs having 0, 1, 2, 3, 4 or 5 amino acid insertions, deletions, or substitutions, or a combination thereof,
wherein the cancer is selected from:
any solid tumor harboring MSI-high alterations;
cervical cancer, in particular PD-L1 positive cervical cancer, more in particular cervical cancer with high expression of PD-L1;
endometrial cancer, in particular MSI-high endometrial cancer;
lung cancer, in particular non-small cell lung cancer (NSCLC), more in particular PD-L1 positive NSCLC, more in particular NSCLC with high expression of PD-L1;
brain cancer, in particular glioblastoma; and
breast cancer, in particular metastatic breast cancer or triple negative breast cancer (TNBC), and
wherein the multispecific antibody is administered in a dose of between 10-1200 mg, preferably between 25-600 or mg 25-300 mg, more preferably between 25-150 mg or 25-100 mg or 25-75 mg, most preferably between 25-50 mg or 50-100 mg.

44. The multispecific antibody for use according to claim 43, wherein the cancer is selected from:
any solid tumor harboring MSI-high alterations;
endometrial cancer, in particular MSI-high endometrial cancer;
lung cancer, in particular non-small cell lung cancer (NSCLC), more in particular NSCLC with high expression of PD-L1; and
breast cancer, in particular metastatic breast cancer or triple negative breast cancer (TNBC), and
wherein the multispecific antibody is administered in a dose of between 10-1200 mg, preferably between 25-600 or mg 25-300 mg, more preferably between 25-150 mg or 25-100 mg, most preferably between 50-100 mg.

45. A method of treating cancer in a subject in need thereof, wherein the cancer is selected from:
any solid tumor harboring MSI-high alterations;
cervical cancer, in particular PD-L1 positive cervical cancer, more in particular cervical cancer with high expression of PD-L1;
endometrial cancer, in particular MSI-high endometrial cancer;
lung cancer, in particular non-small cell lung cancer (NSCLC), more in particular PD-L1 positive NSCLC, more in particular NSCLC with high expression of PD-L1;
brain cancer, in particular glioblastoma; and
breast cancer, in particular metastatic breast cancer or triple negative breast cancer (TNBC),
the method comprising administering between 10-1200 mg, preferably between 25-600 or mg 25-300 mg, more preferably between 25-150 mg or 25-100 mg or 25-75 mg, most preferably between 25-50 mg or 50-100 mg, of a multispecific antibody that comprises a binding domain that binds CD137, comprising:
a variable domain that comprises a CDR1 having an amino acid sequence as set forth in SEQ ID NO: 50, a CDR2 having an amino acid sequence as set forth in SEQ ID NO: 51 and a CDR3 having an amino acid sequence as set forth in SEQ ID NO: 52; or
a variable domain that comprises a CDR1 having an amino acid sequence as set forth in SEQ ID NO: 40, a CDR2 having an amino acid sequence as set forth in SEQ ID NO: 41 and a CDR3 having an amino acid sequence as set forth in SEQ ID NO: 42; or
a variable domain that comprises a CDR1 having an amino acid sequence as set forth in SEQ ID NO: 21, a CDR2 having an amino acid sequence as set forth in SEQ ID NO: 22 and a CDR3 having an amino acid sequence as set forth in SEQ ID NO: 23; or
a variable domain that comprises a CDR1 having an amino acid sequence as set forth in SEQ ID NO: 32, a CDR2 having an amino acid sequence as set forth in SEQ ID NO: 33 and a CDR3 having an amino acid sequence as set forth in SEQ ID NO: 34; and/or
wherein the antibody comprises a binding domain that binds PD-L1, comprising:
a variable domain that comprises a CDR1 having an amino acid sequence as set forth in SEQ ID NO: 68, a CDR2 having an amino acid sequence as set forth in SEQ ID NO: 55 and a CDR3 having an amino acid sequence as set forth in SEQ ID NO: 56; or
a variable domain that comprises a CDR1 having an amino acid sequence as set forth in SEQ ID NO: 93, a CDR2 having an amino acid sequence as set forth in SEQ ID NO: 94 and a CDR3 having an amino acid sequence as set forth in SEQ ID NO: 95; or
a variable domain that comprises a CDR1 having an amino acid sequence as set forth in SEQ ID NO: 93, a CDR2 having an amino acid sequence as set forth in SEQ ID NO: 101 and a CDR3 having an amino acid sequence as set forth in SEQ ID NO: 102; or
a variable domain that comprises a CDR1 having an amino acid sequence as set forth in SEQ ID NO: 90, a CDR2 having an amino acid sequence as set forth in SEQ ID NO: 79 and a CDR3 having an amino acid sequence as set forth in SEQ ID NO: 91,
each of the individual SEQ ID NOs having 0, 1, 2, 3, 4 or 5 amino acid insertions, deletions, or substitutions, or a combination thereof.

46. The method according to claim 45, wherein the cancer is selected from:
any solid tumor harboring MSI-high alterations;
endometrial cancer, in particular MSI-high endometrial cancer;
lung cancer, in particular non-small cell lung cancer (NSCLC), more in particular NSCLC with high expression of PD-L1; and
breast cancer, in particular metastatic breast cancer or triple negative breast cancer (TNBC), and
wherein the multispecific antibody is administered in a dose of between 10-1200 mg, preferably between 25-600 or mg 25-300 mg, more preferably between 25-150 mg or 25-100 mg, most preferably between 50-100 mg.

47. The multispecific antibody for use or method according to any one of claims 37-46, wherein the multispecific antibody is administered intravenously.

48. The multispecific antibody for use or method according to any one of claims 37-47, wherein the multispecific antibody is administered once every two weeks.

49. The multispecific antibody for use or method according to any one of claims 37-48, wherein the multispecific antibody comprises CDR 1, 2 and 3 of MF6797 and CDRs 1, 2 and 3 of MF7702, or variants thereof.

50. The multispecific antibody for use or method according to claim 49, wherein the multispecific antibody comprises SEQ ID NO. 49 and SEQ ID NO. 67, or variants thereof.

51. The multispecific antibody for use or method according to any one of claims 37-48, wherein the multispecific antibody comprises CDR 1, 2 and 3 of MF6783 and CDRs 1, 2 and 3 of MF5542, or variants thereof.

52. The multispecific antibody for use or method according to claim 51, wherein the multispecific antibody comprises SEQ ID NO. 1 and SEQ ID NO. 92, or variants thereof.

53. The multispecific antibody for use or method according to any one of claims 37-48, wherein the multispecific antibody comprises CDR 1, 2 and 3 of MF6754 and CDRs 1, 2 and 3 of MF5561, or variants thereof.

54. The multispecific antibody for use or method according to claim 53, wherein the multispecific antibody comprises SEQ ID NO. 20 and SEQ ID NO. 100, or variants thereof.

55. The multispecific antibody for use or method according to any one of claims 37-48, wherein the multispecific antibody comprises CDR 1, 2 and 3 of MF6785 and CDRs 1, 2 and 3 of MF5439, or variants thereof.

56. The multispecific antibody for use or method according to claim 55, wherein the multispecific antibody comprises SEQ ID NO. 31 and SEQ ID NO. 89, or variants thereof.

57. The multispecific antibody for use or method according to any one of claims 37-48, wherein the multispecific antibody comprises CDR 1, 2 and 3 of MF6795 and CDRs 1, 2 and 3 of MF5442, or variants thereof.

58. The multispecific antibody for use or method according to claim 57, wherein the multispecific antibody comprises SEQ ID NO. 9 and SEQ ID NO. 92, or variants thereof.

59. The multispecific antibody for use or method according to any one of claims 1-58, wherein the antibody comprises a common light chain variable domain having an amino acid sequence as set forth in SEQ ID NO: 110, or a common light chain having an amino acid sequence as set forth in SEQ ID NO: 109, or variants thereof.

60. The multispecific antibody for use or method according to any one of claims 1-59, wherein the antibody comprises a heavy chain constant domain 1 (CH1) having an amino acid sequence as set forth in SEQ ID NO: 112, a heavy chain constant domain 2 (CH2) having an amino acid sequence as set forth in SEQ ID NO: 114, a heavy chain constant domain 3 (CH3) having an amino acid sequence as set forth in SEQ ID NO: 115, and a heavy chain constant domain 3 (CH3) having an amino acid sequence as set forth in SEQ ID NO: 116, or variants thereof.

61. A kit of parts comprising a multispecific antibody as defined in any one of the preceding claims and instructions for use of the multispecific antibody at a dose, or flat dose, of between 25-75 mg or between 25-50 mg or between 25-40 mg or between 25-30 mg, or at 25 mg or 30 mg or 40 mg or 50 mg or 60 mg or 70 mg or 75 mg.

62. The kit of parts according to claim 61, wherein the kit comprises instructions for use of the multispecific antibody in any one the indications selected from the group consisting of:
any solid tumor harboring MSI-high alterations;
cervical cancer, in particular PD-L1 positive cervical cancer, more in particular cervical cancer with high expression of PD-L1;
endometrial cancer, in particular MSI-high endometrial cancer;
lung cancer, in particular non-small cell lung cancer (NSCLC), more in particular PD-L1 positive NSCLC, more in particular NSCLC with high expression of PD-L1;
brain cancer, in particular glioblastoma; and
breast cancer, in particular metastatic breast cancer and triple negative breast cancer (TNBC).
